(19) United States
(12) Patent Application Publication
(10) Pub. No.: US 2022/0397567 A1
(43) Pub. Date: Dec. 15, 2022

(54) NOVEL APPROACH FOR INCREASING CONTRACTILITY IN PATIENTS WITH SYSTOLIC HEART FAILURE

(71) Applicants: THE TRUSTEES OF COLUMBIA UNIVERSITY IN THE CITY OF NEW YORK, New York, NY (US); THE NEW YORK AND PRESBYTERIAN HOSPITAL, New York, NY (US); HARVARD UNIVERSITY, Cambridge, MA (US)

(72) Inventors: Steven O. MARX, New york, NY (US); Alexander KUSHNIR, New York, NY (US); Sergey ZAKHAROV, New York, NY (US); Alexander KATCHMAN, New York, NY (US); Steven P. GYGI, New York, NY (US); Marian KALOCSAY, New York, NY (US); Manu BEN-JOHNY, New York, NY (US); Henry M. COLECRAFT, New York, NY (US); Guoxia LIU, New York, NY (US)

(21) Appl. No.: 17/624,497

(22) PCT Filed: Jul. 2, 2020
(86) PCT No.: PCT/US2020/040695

§ 371 Date: Jan. 3, 2022

Related U.S. Application Data
(60) Provisional application No. 62/869,770, filed on Jul. 2, 2019.
(60) Provisional application No. 62/934,341, filed on Nov. 12, 2019.

(51) Int. Cl. G01N 33/50

(52) U.S. Cl. CPC G01N 33/5044; G01N 33/5041

(57) ABSTRACT

The method for increasing contractility in patients with systolic heart failure involves screening for candidate small molecules which block the interaction between Rad and the plasma membrane and/or block the interaction between Rad and the Ca_{V}1.2/Ca_{V}β2 complex, or between Rad and Ca_{V}β2, in order to increase cardiac contractility. A method for preventing calcium overload and arrhythmias in heart disease involves preventing the dissociation of Rad and the Ca_{V}1.2/Ca_{V}β2 complex, or between Rad and Ca_{V}β2, during beta-adrenergic system activation. Additionally, a method of screening for drugs that block interaction between an RGK GTPase protein and a β-subunit of the calcium channel is provided. A suitable technique, such as fluorescence resonance energy transfer (FRET), may be used to assess blocking of the interaction between the RGK GTPase protein and the β-subunit of the calcium channel for the treatment of heart disease, pain, diabetes, skeletal muscle disorders and/or central nervous system (CNS) disorders.

### CROSS-REFERENCE TO RELATED APPLICATION

This application claims the benefit of U.S. Provisional Patent Application No. 62/934,341, filed on Nov. 12, 2019, and further claims the benefit of U.S. Provisional Patent Application No. 62/869,770, filed on Jul. 2, 2019.

### GOVERNMENT FUNDED RESEARCH

This invention was made with U.S. government support under grant no. 5R01HL121253-06, awarded by the National Institutes of Health. The U.S. government has certain rights to the invention.

### TECHNICAL FIELD

The disclosure of the present patent application relates to the treatment and prevention of cardiovascular disorders, and particularly to a method of screening and providing potential drugs for increasing contractility in patients with systolic heart failure, where the candidate molecules block interaction between Rad and a cardiomyocyte membrane and/or a Ca_{V}1.2/Ca_{V}β2 complex, and/or Ca_{V}β2, and further to a method of screening for drugs that block interaction between an RGK GTPase protein and a cell membrane and/or a voltage calcium channel/Ca_{V}β complex, and/or Ca_{V}β, where blocking of the interaction between the RGK GTPase protein and a cell membrane and/or a voltage calcium channel/Ca_{V}β complex, and/or Ca_{V}β is for the treatment of heart disease, pain, diabetes, skeletal muscle disorders and/or central nervous system (CNS) disorders.

### BACKGROUND ART

Calcium is a critical second messenger in many cell types. Calcium enters into cells through voltage gated calcium channels. By regulating the amount of calcium that enters through these channels, cells can regulate downstream processes.

The strength of cardiac contraction (contractility) is regulated by the concentration of calcium within the cytoplasm of cardiac muscle cells (cardiomyocytes). During systole (cardiac contraction), calcium enters the cytosol from the extracellular space through voltage-gated L-type calcium channels (Ca_{V}1.2) as well as from intrinsic calcium storage compartments (the sarcoplasmic reticulum), through ryanodine receptor calcium channels (RyR2). As cytosolic calcium levels rapidly rise, the calcium binds to the contractile apparatus, enabling the cell to contract. The calcium is then removed from the cytosol and the cell relaxes.

Activation of the beta-adrenergic receptor cascade during exercise and stress causes more calcium to enter the cell through Ca_{V}1.2, resulting in increased contractility and improved cardiac output. However, in certain disease states, such as systolic heart failure, structural heart disease, atrial arrhythmias, ventricular arrhythmias, and catecholaminergic polymorphic ventricular tachycardia (CPVT), activation of the beta-adrenergic receptor system causes dysregulation of cytosolic calcium levels leading to clinical deterioration and death.

Beta-blockers are a ubiquitous class of medications that attenuate the effect of the beta-adrenergic receptor system on the heart, and are a first line treatment for these conditions. Unfortunately, beta-blockers have numerous off-target effects that limit their use and tolerability by patients. Novel agents that specifically block the effects of beta-adrenergic receptor activation on calcium levels in cardiomyocytes may provide important therapeutic potential for many forms of heart disease.

Physiologic β-adrenergic activation of protein kinase A (PKA) during the “fight or flight” response increases Ca²⁺ influx through Ca_{V}1.2 in cardiomyocytes, leading to increased cardiac contractility. In patients with heart disease, however, sustained adrenergic stimulation and the resultant increased Ca²⁺ influx activate maladaptive pro-arrhythmic cellular processes including prolongation of the action potential duration (APD). PKA also acts on other key regulators of excitation-contraction (E-C) coupling, including ryanodine receptors (RyR2), phospholamban, KCNQ1/E1 (I_{Ks}), and troponin I.

Selective attenuation of the incremental Ca_{V}1.2 current that is caused by activation of the β-adrenergic/PKA pathway, while at the same time preserving β-adrenergic stimulation of anti-arrhythmic targets, such as K_{V} channels, which shorten the APD, or SERCA would offer opportunities for novel targeted therapies. Thus, a great deal of research has been performed to define the molecular mechanisms of β-adrenergic regulation of Ca_{V}1.2 to provide tools for the development of targeted therapies. Such research is described, for example, in pending U.S. patent application Ser. No. 16/228,433, filed on Dec. 20, 2018, which is hereby incorporated by reference in its entirety.

Recent research, however, has revealed that protein kinase A (PKA) phosphorylates the Ras-related small G-protein, Rad, and that when Rad is released from the plasma membrane, calcium channel inhibition is decreased, resulting in increased calcium influx and increased contractility. Rad is a member of the RGK family of GTP-binding proteins, is an inhibitor of voltage-gated Ca²⁺ channels, and also is a PKA target. Thus, it would be desirable to find candidate small molecule drugs that block the interaction between Rad and the cardiomyocyte membrane and/or block the interaction between Rad and the Ca_{V}1.2/Ca_{V}β2 complex, and/or block the interaction between Rad and Ca_{V}β2, (or, in the presence of any other Ca_{V}β, block the interaction between Rad and the Ca_{V}1.2/Ca_{V}β complex, and/or between Rad and Ca_{V}(3) in order to increase cardiac contractility. Thus, a method of screening for drugs for increasing contractility in patients with systolic heart failure solving the aforementioned problems is desired.

### DISCLOSURE

Patients with advanced systolic heart failure require urgent treatment with medications called inotropes which act on the heart to temporarily increase contractility and cardiac output. The major classes of inotropes, beta-adrenergic receptor agonists and phosphodiesterase-3 inhibitors, are limited by their effect on the cardiac conduction system, which results in increased heart rate or sinus tachycardia, and effects on systemic vasculature, which results in vasodilation and hypotension. There is an unmet clinical need for a drug that selectively increases cellular calcium levels in cardiac muscle cells without affecting the cardiac conduction system or systemic vasculature.

It has been found that binding between Ca_{V}1.2 and Ca_{V}β2 is a critical mediator of the effects of beta-adrenergic receptor activation on calcium handling in cardiomyocytes. Therefore, drugs that block the interaction between Ca_{V}1.2 and Ca_{V}β2 are expected to have a therapeutic effect in patients with cardiovascular conditions due to abnormalities in intracellular calcium handling (e.g., chronic heart failure, arrhythmias, catecholaminergic polymorphic ventricular tachycardia). It has been further found that PKA phosphorylates Rad, and that when Rad is released from Ca_{V}β2 and/or from the plasma membrane, calcium channel inhibition is decreased, resulting in increased calcium influx and increased contractility. Rad is a member of the RGK family of GTP-binding proteins, is an inhibitor of voltage-gated Ca²⁺ channels and is also a PKA target. Thus, candidate small molecule drugs should block the interaction between Rad and the cardiomyocyte membrane and/or block the interaction between Rad and the Ca_{V}1.2/Ca_{V}β2 complex and/or Ca_{V}β2, (or, in the presence of any other Ca_{V}β, block the interaction between Rad and the Ca_{V}1.2/Ca_{V}β complex, and/or between Rad and Ca_{V}(3), in order to increase cardiac contractility.

It should be understood that, for purposes of this discussion, any reference to, for example, the interaction between Rad and the Ca_{V}1.2/Ca_{V}β2 complex and/or Ca_{V}β2, is intended to apply equally to any other Ca_{V}1.2/Ca_{V}β complex and/or Ca_{V}β and/or Ca_{V} in other tissues/cell types where the intention is to alter Ca²⁺ influx through Ca_{V} by altering Rad binding.

Accordingly, this technology identifies the Rad protein as a therapeutic target for selectively increasing or decreasing calcium levels in cardiac muscle cells without affecting the heart rate. Rad binds to the cell membrane and to the Ca_{V}1.2/Ca_{V}β2 complex and/or Ca_{V}β2, resulting in basal inhibition of the calcium channel. Phosphorylation of Rad results in its dissociation, leading to increased calcium entry into the cell. Therefore, therapeutics that can disrupt the interaction between Rad and the cell membrane, the Ca_{V}1.2/Ca_{V}β2 complex, and/or Ca_{V}β2, should lead to effective inotropes for patients with advanced heart failure. Alternatively, agents that maintain Rad binding to Ca_{V}β2, Ca_{V}1.2/Ca_{V}β2 complex, and/or cell membrane, despite PKA activation would decrease Ca²⁺ entry into the cell and may prevent Ca²⁺ overload which can cause arrhythmias.

The method of screening for drugs for increasing contractility in patients with systolic heart failure involves screening for candidate small molecules which block the interaction between Rad and the cardiomyocyte membrane, and/or block the interaction between Rad and the Ca_{V}1.2/Ca_{V}β2 complex and/or Ca_{V}β2, in order to increase cardiac contractility.

The method of screening for drugs for decreasing Ca²⁺ entry into cells involves screening for candidate small molecules which prevent Rad from dissociating from the cardiomyocyte membrane, and/or from the Ca_{V}1.2/Ca_{V}β2 complex and/or from Ca_{V}β2, in order to prevent Ca²⁺ overload.

The method of screening for drugs that increase Ca_{V}1.2 calcium current in cardiomyocytes and increase cardiac contractility may begin with generation of a stable cell line that expresses Ca_{V}1.2 and Ca_{V}β2. The stable cell line is transiently transfected with Rad and a light sensitive ion channel A calcium sensitive fluorescent dye is added to the stable cell line, and the stable cell line is exposed to light to depolarize each of the cells and activate the Ca_{V}1.2 calcium channels. An amplitude of calcium current is assessed by measuring an intensity of calcium dye fluorescence. The experiment is performed in the absence and presence of screening compounds. A compound that interferes with the interaction between Rad and the Ca_{V}1.2 and Ca_{V}β2 complex and/or Rad and the membrane, and/or between Rad and Ca_{V}β2, will result, for example, in disinhibited Ca_{V}1.2 channels and therefore a larger calcium current/calcium fluorescence in response to cellular depolarization. Thus, it can be determined that the screening compound increases, for example, Ca_{V}1.2 calcium current in cardiomyocytes and increases cardiac contractility if the intensity of calcium dye fluorescence is above a threshold value.

Rad also inhibits Ca_{V}2.2, a neuronal isoform of the L-type calcium channel Ca_{V}2.2 regulates calcium current in neurons involved in pain and epilepsy, and is a clinically validated target for treating chronic pain. Ca_{V}2.2 may be regulated by Ca_{V}β2, Ca_{V}(33, or Ca_{V}(34 molecules that either block or enhance the interaction between Rad and the Ca_{V}2.2/Ca_{V}β complex may have therapeutic potential for treating chronic pain or epilepsy.

Additionally, a method of screening for drugs that block interaction between an RGK GTPase protein and a β-subunit of the calcium channel is provided. A suitable technique, such as fluorescence resonance energy transfer (FRET), may be used to assess blocking of the interaction between the RGK GTPase protein and the β-subunit of the calcium channel Blocking of the interaction between the RGK GTPase protein and the β-subunit of the calcium channel is for the treatment of heart disease, pain, diabetes, skeletal muscle disorders and/or central nervous system (CNS) disorders.

Additionally, a method of screening for drugs that maintain the interaction between an RGK GTPase protein and a β-subunit of the calcium channel in the presence of activated PKA is provided. A suitable technique, such as fluorescence resonance energy transfer (FRET), may be used to assess maintaining the interaction between the RGK GTPase protein and the β-subunit of the calcium channel. Maintaining the interaction between the RGK GTPase protein and the β-subunit of the calcium channel is for the treatment of heart disease, pain, diabetes, skeletal muscle disorders and/or central nervous system (CNS) disorders.

The RGK GTPase family of proteins includes Rad, Rem, Rem2, and Kir/Gem, the β-subunit may be CACNB1, CACNB2, CACNB3, or CACNB4, and the calcium channel may be CACNA1S, CACNA1C, CACNA1D, CACNA1F, CACNA1A, CACNA1B, CACNA1E, CACNA1G, CACNA1H, or CACNA1I. Using FRET to assess blocking of the interaction between the RGK GTPase protein and the β-subunit of the calcium channel is implemented by attaching a first fluorophore to the RGK GTPase protein and attaching a second fluorophore to the β-subunit of a calcium channel. One of the fluorophores is then excited, and emission from the second fluorophore is measured. This is known as FRET efficiency. The FRET efficiency is measured to determine the interaction between the RGK GTPase protein and the β-subunit of a calcium channel.

A method of screening for drugs that increase α_{1C} calcium current in cardiomyocytes and increase cardiac contractility is further provided. The screening method includes the step of screening a molecular library for one or more candidate molecules which block interaction between Rad and a CaV1.2/CaVβ2 complex or between Rad and CaVβ2. In a further embodiment, a method of screening for drugs that increase α_{1C} calcium current in cardiomyocytes and increase cardiac contractility is also provided, including the step of screening a molecular library for one or more candidate molecules which block interaction between Rad and CaVβ2.

Additionally, a method of screening for drugs that enhance interaction between an RGK GTPase protein and a β-subunit of a calcium channel to decrease calcium current, reduce calcium overload and reduce arrhythmias is further provided. The method includes the steps of: a) attaching a first fluorophore to an RGK GTPase protein; b) attaching a second fluorophore to a β-subunit of a calcium channel; c) expressing the RGK GTPase protein and the β-subunit of the calcium channel in a cell line; d) expressing a catalytic subunit of PKA in the cell line; e) exciting one of the first and second fluorophores; and f) measuring fluorescence resonance energy transfer (FRET) efficiency to determine interaction between the RGK GTPase protein and the β-subunit of the calcium channel.

In an additional alternative embodiment, a method of screening for drugs that block interaction between an RGK GTPase protein and a β-subunit of a calcium channel is also provided. The method includes the steps of: a) attaching a first fluorophore to an RGK GTPase protein; b) attaching a second fluorophore to an integral membrane bound protein; c) exciting one of the first and second fluorophores; and d) measuring fluorescence resonance energy transfer (FRET) efficiency to determine interaction between the RGK GTPase protein and the integral membrane bound protein.

These and other features of the present subject matter will become readily apparent upon further review of the following specification.

### BRIEF DESCRIPTION OF THE DRAWINGS

FIG. 1A diagrammatically illustrates α_{1C} and β₂ at a cardiomyocyte membrane, without Rad.

FIG. 1B is a graph showing Ba²⁺ current elicited by voltage ramp every 10 s from −60 mV to +60 mV over 200 ms, in the absence of Rad. The black traces show current elicited without forskolin. The blue traces show current elicited after administration of forskolin (i.e., a downstream mimic of beta-adrenergic receptor activation).

FIG. 1C is a diary plot of normalized I_{Ba} amplitude at +10 mV in the absence of Rad, where the black plot shows the control I_{Ba} amplitude and the blue plot shows the I_{Ba} amplitude after forskolin.

FIG. 1D diagrammatically illustrates α_{1C}, β₂ and Rad at a cardiomyocyte membrane.

FIG. 1E is a graph showing Ba²⁺ current elicited by voltage ramp every 10 s from −60 mV to +60 mV over 200 ms, with Rad present. The black traces show current elicited before forskolin. The blue traces show current elicited after forskolin (a downstream mimic of beta-adrenergic receptor activation).

FIG. 1F is a diary plot of normalized I_{Ba} amplitude at +10 mV with Rad present, where the black plot shows the control I_{Ba} amplitude and the blue plot shows the I_{ba} amplitude after forskolin.

FIG. 1G diagrammatically illustrates α_{1C}, β₂ and S/A mutant Rad at a cardiomyocyte membrane.

FIG. 1H is a graph showing Ba²⁺ current elicited by voltage ramp every 10 s from −60 mV to +60 mV over 200 ms, with 4SA mutant Rad. The black traces show current elicited before forskolin. The blue traces show current elicited after forskolin (a downstream mimic of beta-adrenergic receptor activation).

FIG. 1I is a graph showing Ba²⁺ current elicited by voltage ramp every 10 s from −60 mV to +60 mV over 200 ms, with 2SA mutant Rad. The black traces show current elicited before forskolin. The blue traces show current elicited after forskolin (a downstream mimic of beta-adrenergic receptor activation).

FIG. 1J is a combined bar and column scatter plot showing the fold increase in peak current caused by forskolin. The bar graphs are mean±SEM with P<0.0001 by one-way Anova; ** P<0.01, **** P<0.0001 by Tukey's multiple comparison test. The combined bar and column scatter plot compares the cases with no Rad (n=27); WT Rad (n=76); 14SA mutant Rad (n=9); 4 SA mutant Rad (n=23); and 2SA mutant Rad (n=18).

FIG. 1K is a combined bar and column scatter plot showing Boltzmann function parameters V_{mid}. The bar graphs show ** P<0.01 by unpaired t-test. The combined bar and column scatter plot compares the cases with no Rad (n=15); WT Rad (n=16); 4SA mutant Rad (n=8); and 2SA mutant Rad (n=13).

FIG. 2A shows a series of graphs with conductance of a single Cav1.2 channel for the case of no Rad. The black plots illustrate results of a control and the blue plots illustrate channel activity in the presence of active PKA (PKA catalytic subunit). The upper four graphs are stochastic records, where channel closures correspond to the zero-current portions of the trace (on horizontal gray lines) and openings correspond to downward deflections to the open level (slanted gray curves). Averaging many records yields a mean current that can be divided into the open level (slanted gray curve) to furnish the Pₒ versus voltage relation, which is shown in the sigmoidal trace in the bottom graph, which is averaged over multiple patches. In all experiments, α_{1C} and β₂ were expressed in HEK cells with no Rad, in the absence or presence of exogenous PKA catalytic subunit.

FIG. 2B shows a series of graphs for the case of WT Rad. The black plots illustrate results of a control and the blue plots illustrate channel activity in the presence of active PKA (PKA catalytic subunit). The upper four graphs are stochastic records, where channel closures correspond to the zero-current portions of the trace (on horizontal gray lines) and openings correspond to downward deflections to the open level (slanted gray curves). Averaging many records yields a mean current that can be divided into the open level (slanted gray curve) to furnish the Pₒ versus voltage relation, which is shown in the sigmoidal trace in the bottom graph, which is averaged over multiple patches. In all experiments, α_{1C} and β₂ were expressed in HEK cells with WT Rad, in the absence or presence of exogenous PKA catalytic subunit.

FIG. 2C shows a series of graphs for the case of 4SA-mutant Rad. The black plots illustrate results of a control and the blue plots illustrate a PKA catalytic case. The upper four graphs are stochastic records, where channel closures correspond to the zero-current portions of the trace (on horizontal gray lines) and openings correspond to downward deflections to the open level (slanted gray curves). Averaging many records yields a mean current that can be divided into the open level (slanted gray curve) to furnish the Pₒ versus voltage relation, which is shown in the sigmoidal trace in the bottom graph, which is averaged over multiple patches. In all experiments, α_{1C} and β₂ were expressed in HEK cells with 4SA-mutant Rad, in the absence or presence of exogenous PKA catalytic subunit.

FIG. 3A is a graph showing Ba²⁺ current of Ca_{V}1.2 channels co-expressed with mutant Rad unable to bind β2-subunit elicited by voltage ramp every 10 s from −60 mV to +60 mV over 200 ms. The black traces represent the date before forskolin and the blue traces show the data after forskolin. This illustrates the interaction between Rad and the β2 subunit is required for forskolin-induced stimulation of Ca²⁺ channel currents.

FIG. 3B is a graph showing Ba²⁺ current of Ca_{V}1.2 channels co-expressed with mutant β2 that is unable to bind Rad elicited by voltage ramp every 10 s from −60 mV to +60 mV over 200 ms. The black traces represent the data before forskolin and the blue traces show the data after forskolin. This illustrates the interaction between Rad and the β2 subunit is required for forskolin-induced stimulation of Ca²⁺ channel currents.

FIG. 3C is a combined bar and column scatter plot showing the fold increase in peak current caused by forskolin. The bar graphs are mean+SEM, with P<0.0001 by one-way Anova; ** P<0.01, and *** P<0.001 by Tukey's multiple comparison test. FIG. 3C compares the cases of Rad and n=76 (see also FIG. 1J); Rad mutant and n=20; and a mutant and n=15.

FIG. 3D is a combined bar and column scatter plot showing the Boltzmann function parameters V_{mid}. The bar graphs are P<0.01 by unpaired t-test. FIG. 3D compares the cases of Rad and n=16 (see also FIG. 1K); Rad mutant and n=19; and a mutant and n=13.

FIG. 3E is a graph showing Ba²⁺ current of Ca_{V}2.2 channels elicited by voltage ramp every 10 s from −60 mV to +60 mV over 200 ms. The black traces represent the data before forskolin and the blue traces show the data after forskolin.

FIG. 3F is a combined bar and column scatter plot showing the fold increase in peak current caused by forskolin. The bar graphs are mean+SEM.

FIG. 3G is a combined bar and column scatter plot showing the Boltzmann function parameters V_{mid}. The bar graphs are P<0.001 by unpaired t-test for n=5.

FIG. 4 shows confocal images of rat cardiomyocytes infected with CFP-Rad adenovirus, before and 5 minutes after superfusion of isoproterenol, respectively. This demonstrates that beta adrenergic receptor activation results in migration of Rad away from the membrane and into the cytosol.

FIG. 5A diagrammatically shows a stable cell line that expresses α_{1C} and β₂. These cells are then transiently transfected with Rad, as well as a light sensitive ion channel.

FIG. 5B diagrammatically shows a calcium sensitive fluorescent dye added to the cells of FIG. 5A.

FIG. 5C diagrammatically shows exposure of the cells of FIG. 5B to light, resulting in depolarization of the cell and activation of α_{1C} calcium channels. The amplitude of calcium current can be assessed via the intensity of calcium dye fluorescence.

FIG. 6A and FIG. 6B are schematics showing protein labeling and workflow for isolated cardiomyocytes (CMs), where “BP” represents biotin-phenol, “Iso” represents 1 μM isoproterenol, and phospholamban (PLB) phosphorylation was assessed using phospho-specific PLB antibody.

FIG. 6C is a volcano plot for ten minute isoproterenol-treated α_{1C}-APEX2 samples relative to no isoproterenol. The data shown are means for five pairs of samples, where the P values were Benjamini-Hochberg corrected, Rad (shown in red) is reduced by 50% and the PKA catalytic subunit (shown in green) is increased by 50%.

FIG. 6D is a volcano plot for ten minute isoproterenol-treated β_{1b}-APEX2 mice relative to no isoproterenol. The data shown are means for three pairs of samples, where the P values were Benjamini-Hochberg corrected, Rad (shown in red) is reduced by 30% and the PKA catalytic subunit (shown in green) is increased by 68%.

FIG. 6E and FIG. 6F are schematics showing protein labeling and workflow for isolated cardiomyocytes from non-transgenic (NTG) mice. Cell lysates were assessed for phosphorylation of PLB by immunoblot.

FIG. 6G is a volcano plot for ten minute isoproterenol-treated NTG mice cardiomyocytes relative to no isoproterenol. The data shown are means for four pairs of samples. P values were Benjamini-Hochberg corrected, and Rad (shown in red) is unchanged.

FIG. 6H, FIG. 6I and FIG. 6J are schematics showing protein labeling and workflow for Langendorff-perfused α_{1C}-APEX2 mouse hearts. An electrocardiogram confirmed viability of the hearts and response to isoproterenol, where downstream PKA signaling was confirmed by phosphorylation of PLB, and “BPM” represents beats per minute.

FIG. 6K is a volcano plot for five minute isoproterenol-treated α_{1C}-APEX2 mouse hearts relative to no isoproterenol. The data shown are means for four pairs of samples, where the P values were Benjamin-Hochberg corrected.

FIG. 7A schematically illustrates rabbit cardiac α_{1C} and β subunits.

FIG. 7B schematically illustrates the binary transgene system, where the expression of reverse tetracycline-controlled transactivator (rtTA) is driven by the cardiac-specific α-myosin heavy chain promoter.

FIG. 7C is an exemplary graph showing whole-cell Ca_{V}1.2 currents of 35-mutant α_{1C} transgenic mice cardiomyocytes.

FIG. 7D is a graph showing fold changes (iso vs. no iso) of peak Ca²⁺ current (I_{ca}) at 0 mV caused by isoproterenol (shown as closed circles) or forskolin (shown as open circles) for pWT α_{1C} and 35-mutant α_{1C} channels.

FIG. 7E is a fluorescent image of isolated cardiomyocytes expressing GFP-tagged 28-β mutant.

FIG. 7F shows an anti-13 antibody immunoblot of cleared lysates from doxycycline-fed 35-mutant α_{1C} transgenic mice or 35-mutant α_{1C} X GFP-tagged 28-mutant β_{2B} expressing mice hearts.

FIG. 7G shows anti-FLAG antibody (upper blot) and anti-13 antibody (lower blot) immunoblots of anti-FLAG antibody immunoprecipitations from cleared lysates of hearts from pWT, 35-α and 35-α X GFP-tagged-28-β expressing mice.

FIG. 7H is a graph showing exemplary whole-cell Ca_{V}1.2 currents of GFP-tagged-28-mutant β_{2B} transgenic mice cardiomyocytes.

FIG. 7I is a graph showing exemplary whole-cell Ca_{V}1.2 currents recorded from 35-mutant α_{1C} X 28-mutant β_{2B} transgenic mice cardiomyocytes.

FIG. 7J is a graph showing the fold changes (iso vs. no iso) at 0 mV in peak Ca²⁺ current caused by isoproterenol (shown as closed circles) or forskolin (shown as open circles) for cardiomyocytes isolated from transgenic mice expressing GFP-tagged WT β_{2B} subunit, GFP-tagged 28-mutant β_{2B}, or both 35-mutant α_{1C} and GFP-tagged 28-mutant β_{2B}.

FIG. 8A is a graph showing exemplary current-voltage relationship of Ca²⁺ currents from α_{1C}-APEX2 mice cardiomyocytes acquired in the absence (black trace) and presence of 300 nM nisoldipine (red trace).

FIG. 8B shows the time course of changes in sarcomere length after superfusion of nisoldipine (300 nM) containing solution. Cardiomyocytes were field-stimulated at 1-Hz.

FIG. 8C is a bar graph of percent (%) shortening in the absence and presence of nisoldipine.

FIG. 8D shows a streptavidin-HRP blot of lysates of isolated ventricular cardiomyocytes from α_{1C}-APEX2 and β_{2B}-APEX2 transgenic mice.

FIG. 8E shows the anti-V5 antibody/Alexa 594 fluorescence and streptavidin-Alexa 488 fluorescence of cardiomyocytes isolated from α_{1C}-APEX2 and β_{2B}-APEX2 expressing mice exposed to biotin-phenol and H₂O₂ or no H₂O₂.

FIG. 8F shows the streptavidin-Alexa 488 fluorescence of tissue sections of Langendorff-perfused α_{1C}-APEX2 heart.

FIG. 8G shows immunoblots of biotin-labeled proteins from α_{1C}-APEX2 and β_{2B}-APEX2 mice cardiomyocytes.

FIG. 8H shows proteins with a ratio of >2 (measured by normalized TMT signal/noise) in experimental conditions compared to a no-labeling control (i.e., no H₂O₂) sorted by spectral counts.

FIG. 9A schematically shows protein labeling and workflow for isolated cardiomyocytes (CM).

FIG. 9B schematically illustrates protein labeling and workflow for Langendorff-perfused α_{1C}-APEX2 mice hearts.

FIG. 9C schematically illustrates protein labeling and workflow for isolated cardiomyocytes from non-transgenic (NTG) mice.

FIG. 9D is a volcano plot of fold-change of iso vs. no iso for relative protein quantification by TMT mass spectrometry of proteins isolated from non-transgenic (NTG) mice cardiomyocytes.

FIG. 10A shows exemplary traces of Ba²⁺ currents of 35-mutant α_{1C}+28-mutant β_{2B} in the absence and in the presence of Rad elicited by voltage ramp every 10 s from −60 mV to +60 mV over 200 ms.

FIG. 10B is a graph showing a fold increase in maximum conductance (Gₘₐₓ) induced by forskolin for 35-mutant α_{1C}+28-mutant β_{2B} in the absence and presence of Rad.

FIG. 10C is a graph showing the Boltzmann function parameter V_{mid}.

FIG. 11A is a plot showing the fold changes (forskolin vs. no forskolin) in Gₘₐₓ, where the data for Rad is the same as that for FIG. 1J.

FIG. 11B illustrates FRET 2-hybrid binding isotherms determined for Cerulean (Cer)-tagged β_{2B} and N-terminal Venus (Ven)-tagged WT Rad. FRET efficiency (E_{D}) is plotted against the free concentration of Ven-WT.

FIG. 11C illustrates FRET 2-hybrid binding isotherms determined for Cerulean (Cer)-tagged β_{2B} and N-terminal Venus (Ven)-tagged 4-SA mutant Rad. FRET efficiency (E_{D}) is plotted against the free concentration of Ven-4SA-mutant Rad.

FIG. 11D is a graph showing Ba²⁺ current of Ca_{V}1.3 channels without or with expression of Rad elicited by voltage ramp every 10 s from −60 mV to +60 mV over 200 MS.

FIG. 11E is a graph showing fold changes (forskolin vs. no forskolin) in Gₘₐₓ.

FIG. 11F is a graph showing the Boltzmann function parameter V₅₀.

FIG. 11G is a graph showing Ba²⁺ current of Ca_{V}2.2 channels without or with expression of Rad or Rem elicited by voltage ramp every 10 s from −60 mV to +60 mV over 200 ms.

FIG. 11H is a graph showing fold changes (forskolin vs. no forskolin) in Gₘₐₓ.

FIG. 11I is a graph showing the Boltzmann function parameter V₅₀.

FIG. 11J schematically illustrates β-adrenergic regulation of Ca²⁺ channels.

FIG. 11K schematically illustrates β-adrenergic regulation of Ca²⁺ channels.

FIG. 12A is a table showing the 35 putative PKA phosphorylation sites in rabbit α_{1C}.

FIG. 12B is a combined bar and column scatter plot of Boltzmann function parameters V₅₀. Left to right: WT Cav1.2; all 35 phosphorylation sites on Cav1.2 removed; all 28 phosphorylation sites on β_{2B} removed; combination of all 35 phosphorylation sites on Cav1.2 and all 28 phosphorylation sites on β_{2B} removed.

FIG. 12C is a graph of isoproterenol and forskolin-induced increase in nisoldipine-resistant current stratified by total basal current density before application of nisoldipine.

FIG. 12D is a table showing the 28 putative PKA phosphorylation sites in the N-terminal (NT), Hook, GK and C-terminal (CT) domains of β_{2B}.

FIG. 13A illustrates the prefuse force directed map of proteins from FIG. 8H.

FIG. 13B is a table showing the GO-term (cellular localization) enrichment for the proteins of FIG. 8H.

FIG. 14A is an exemplary graph showing whole-cell Ca_{V}1.2 currents recorded from freshly dissociated cardiomyocytes of α_{1C}-APEX2 transgenic mice.

FIG. 14B is an exemplary graph showing whole-cell Ca_{V}1.2 currents recorded from freshly dissociated cardiomyocytes of β_{2B}-APEX2 transgenic mice.

FIG. 14C shows Western blots probed with phospho-phospholamban antibody of isolated cardiomyocytes exposed to 1 μM isoproterenol. For cardiomyocytes isolated from α_{1C}-APEX2 and β_{2B}-APEX2 mice, the cardiomyocytes were exposed to isoproterenol after incubation with biotin-phenol.

FIG. 14D shows a Western blot probed with phospho-phospholamban antibody for a whole heart exposed to 1 μM isoproterenol for 5 minutes after infusion of biotin-phenol.

FIG. 15A is a dendrogram showing two-way hierarchical clustering of scaled TMT signal to noise (s/n) data for streptavidin-purified proteins from α_{1C}-APEX2 expressing cardiomyocytes under isoproterenol stimulation (iso) or with vehicle (no iso).

FIG. 15B is a dendrogram showing two-way hierarchical clustering of scaled TMT signal to noise (s/n) data for streptavidin-purified proteins from α_{1C}-APEX2 expressing cardiomyocytes under isoproterenol stimulation (iso) or with vehicle (no iso).

FIG. 15C is a dendrogram scaled with relative quantification data for 2,610 proteins from whole-organ α_{1C}-APEX without or with perfusion of isoproterenol, related to that of FIG. 9D, used for two-way hierarchical clustering.

FIG. 15D is a dendrogram showing two-way hierarchical clustering of scaled TMT signal to noise (s/n) data from non-transgenic (NTG) cardiomyocytes under isoproterenol stimulation (iso) or with vehicle (no iso). Scaled data of 4,622 quantified proteins from a biological quadruplicate experiment are shown.

FIG. 16A shows the MS² spectrum and TMT quantification parameters of a Rad peptide changed upon isoproterenol treatment of murine hearts. The MS² spectrum is used for identification of the peptide IFGGIEDGPEAEAAGHTYDR mapping to Rad. The y and b ion m/z identified in the spectrum and their deviation from theoretical m/z are displayed on the left of FIG. 16A. FIG. 16A additionally shows the ion injection times, isolation specificity, sum of signal to noise (SN) over all TMT channels, TMT raw intensities, adjusted intensities and final SN intensities used for relative quantification as well as SPS ion m/z.

FIG. 16B is a table showing gene names of proteins with FDR-corrected P<0.05 for the following three approaches: 1) cardiomyocytes isolated from α_{1C}-APEX and 2) β_{2B}-APEX mice, and 3) α_{1C}-APEX hearts.

FIG. 16C shows a Venn diagram of the data from FIG. 16B.

FIG. 17A is an exemplary graph showing whole-cell Ca_{V}1.2 currents elicited from step depolarizations recorded from HEK cells expressing Rad.

FIG. 17B illustrates the methodology used for generating G/Voltage curves. For sub-graph (i), upper: 200 ms voltage ramp from −60 mV to +60 mV was applied every 10 s, and shown in the lower graph: current traces with an average of 3 traces before forskolin (black traces) and 3 traces after forskolin (blue traces). For sub-graph (ii), the data was processed to convert time scale to applied voltage and corrected for junction potential (10 mV). For sub-graph (iii), the data were processed as described below, with regard to the methods to convert Ba²⁺ current to conductance.

FIG. 17C is a graph of forskolin-induced fold changes in current stratified by current density before forskolin.

FIG. 17D is a graph showing the voltage-dependence of forskolin-induced augmentation of Ba²⁺ current, showing the ratio of peak current after forskolin application to peak current before forskolin application for cells transfected without and with Rad, where the cells were transfected with WT α_{1C} and WT β_{2B}.

FIG. 17E is a graph showing the voltage-dependence of forskolin-induced augmentation of Ba²⁺ current, showing the ratio of peak current after forskolin application to peak current before forskolin application for cells transfected without and with Rad, where the cells were transfected with 35-mutant α_{1C} and 28-mutant β_{2B}.

FIG. 17F is a histogram showing the distribution of sweep-by-sweep average P_{O} (single trial <P_{O}>) for Ca_{V}1.2 without Rad.

FIG. 17G is a graph showing the distribution of sweep-by-sweep average P_{O} (single trial <P_{O}>) for Ca_{V}1.2 without Rad, specifically showing the conditional Pₒ-voltage relationship obtained for sweeps exhibiting high activity.

FIG. 17H is a histogram showing the distribution of sweep-by-sweep average P_{O} (single trial <P_{O}>) for Ca_{V}1.2 with Rad, specifically showing the fraction of blank sweeps is markedly increased (90%) with expression of Rad.

FIG. 17I is a histogram showing the distribution of sweep-by-sweep average P_{O} (single trial <P_{O}>) for Ca_{V}1.2 with Rad and the PKA catalytic subunit co-expressed.

FIG. 17J is a graph showing the distribution of sweep-by-sweep average P_{O} (single trial <P_{O}>) for Ca_{V}1.2 with Rad and the PKA catalytic subunit co-expressed, specifically showing the conditional Pₒ-voltage relationship computed only from sweeps exhibiting high activity.

FIG. 18A shows the genetic code for Rad with all the serine/Threonine residues that were mutated to alanine highlighted.

FIG. 18B shows the mass spectrometry identification of phosphorylated residues on Rad enriched with an anti-GFP nanobody matrix from HEK cells expressing GFP-Rad and treated with forskolin.

FIG. 18C illustrates phosphorylation sites identified previously in Rad.

FIG. 18D shows the genetic code for serine residues mutated to alanine in 4-SA (mutant indicated by arrows).

FIG. 19A shows the genetic code for alanine substitutions of Rad at residues R208 and L235.

FIG. 19B shows the genetic code for alanine substitutions at residues D244, D320 and D322 of β_{2B}, which were created to attenuate Rad binding to β.

FIG. 19C is a graph showing the Ba²⁺ current of Ca_{V}1.2 channels elicited by voltage ramp every 10 s from −60 mV to +60 mV over 200 ms.

FIG. 19D is a graph showing the Boltzmann function parameter V₅₀. The data for Rad is the same as that of FIG. 1K.

FIG. 19E is a graph showing the fold changes (forskolin vs. no forskolin, respectively) in Gₘₐₓ for Cav1.3. The data for WT Rad and WT β_{2B} is same as that of FIG. 11E.

FIG. 19F is a graph showing the fold changes (forskolin vs. no forskolin, respectively) in Gₘₐₓ for Cav2.2. The data for WT Rad and WT β_{2B} is same as that of FIG. 11H.

FIG. 20A illustrates FRET 2-hybrid binding isotherms determined for Cerulean (Cer)-tagged β₃, and N-terminal Venus (Ven)-tagged WT or 4-SA mutant Rad, where FRET efficiency (E_{D}) is plotted against the free concentration of Ven-WT or Ven-4SA-mutant Rad, respectively.

FIG. 20B illustrates FRET 2-hybrid binding isotherms determined for Cerulean (Cer)-tagged β₄, and N-terminal Venus (Ven)-tagged WT or 4-SA mutant Rad, where FRET efficiency (E_{D}) is plotted against the free concentration of Ven-WT or Ven-4SA-mutant Rad, respectively.

FIG. 20C is a bar graph summarizing mean K_{d,EFF} for β_{2B}, β₃ and β₄, with WT or 4-SA mutant Rad, expressed without and with the catalytic PKA subunit.

FIG. 21A shows the ClustalW alignment of murine Rad, Rem1, Rem2, and Gem sequences, with phosphorylation sites for mouse Rad Ser²⁵, Ser³⁸, Ser²⁷² and Ser³⁰⁰ being indicated with arrows. The blue highlights are basic amino acids (Arg, Lys and His), and the red highlights are Ser and Thr.

FIG. 21B shows the ClustalW alignment of Rad sequences, with conserved phosphorylation sites for mouse Ser²⁵, Ser³⁸, Ser²⁷² and Ser³⁰⁰ being shown. The blue highlights are basic amino acids (Arg, Lys and His), and the red highlights are Ser and Thr.

Similar reference characters denote corresponding features consistently throughout the attached drawings.

### BEST MODE(S)

As discussed above, a great deal of research has been performed to define the molecular mechanisms of β-adrenergic regulation of Ca_{V}1.2 to provide tools for the development of targeted therapies. Such research is described, for example, in pending U.S. patent application Ser. No. 16/228,433, filed on Dec. 20, 2018, which is hereby incorporated by reference in its entirety. It has been further found that PKA phosphorylates Rad, and that when Rad is released from Ca_{V}β2 and/or the plasma membrane, and/or somewhere else on the Ca_{V}1.2/Ca_{V}β2 complex. As noted above, references to, for example the interaction between Rad and the Ca_{V}1.2/Ca_{V}β2 complex and/or Ca_{V}β2, apply equally to any other Ca_{V}/Ca_{V}β complex and/or Ca_{V}(3.

This results in decreased Cav1.2 calcium channel inhibition, resulting in increased calcium influx and increased contractility. Rad is a member of the RGK family of GTP-binding proteins, is an inhibitor of voltage-gated Ca²⁺ channels and is also a PKA target. Thus, candidate small molecule drugs should block the interaction between Rad and the cardiomyocyte membrane, and/or block the interaction between Rad and Ca_{V}β2, and/or otherwise block the interaction between Rad and the Ca_{V}1.2/Ca_{V}β2 complex, in order to increase cardiac contractility.

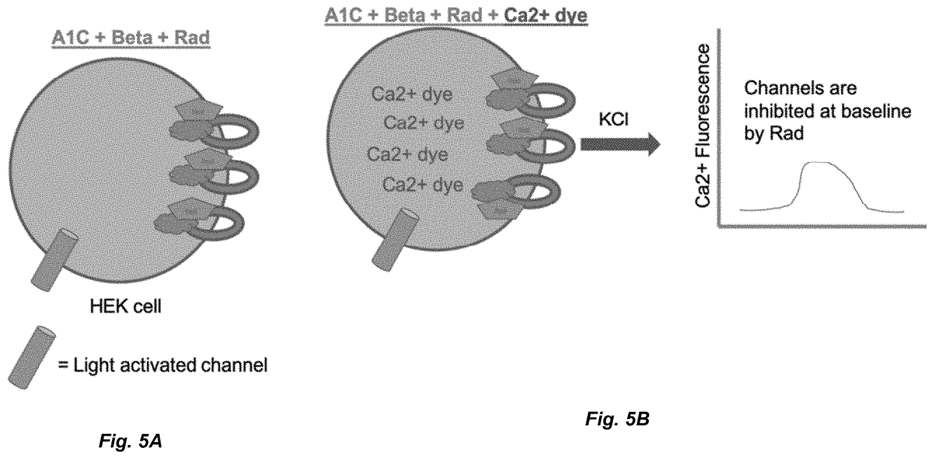

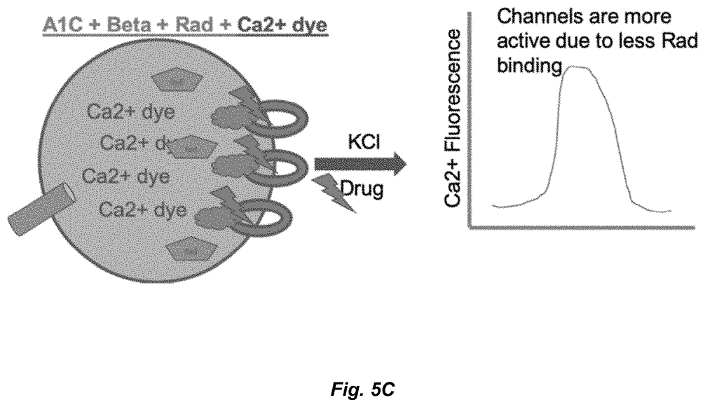

FIGS. 5A-5C diagrammatically illustrate the method of screening compounds that block the interaction between Rad and the α_{1C}/β₂ complex and/or Rad and the membrane. FIG. 5A diagrammatically shows a stable cell line that expresses α_{1C} and β2. These cells are then transiently transfected with Rad, as well as a light sensitive ion channel. In FIG. 5B, a calcium sensitive fluorescent dye added to the cells of FIG. 5A. FIG. 5C shows exposure of the cells of FIG. 5B to light, resulting in depolarization of the cell and activation of α_{1C} calcium channels. The amplitude of calcium current can be assessed via the intensity of calcium dye fluorescence. This experiment is performed in the absence and presence of screening compounds. A compound that interferes with the interaction between Rad and the α_{1C}/β₂ complex and/or Rad and the membrane will result in disinhibited α_{1C} channels and, therefore, a larger calcium current/calcium fluorescence in response to cellular depolarization.

Preliminary research involving mass spectrometry of the Ca_{V}1.2 interactome in adult cardiomyocytes has been performed. To identify new candidate regulators, transgenic (TG) mice were created with doxycycline-inducible expression of ascorbate-peroxidase (APEX2) conjugated to α_{1C} or β₂, enabling in situ biotin-labeling and subsequent identification of Ca_{V}1.2 near-neighbors by mass spectrometry (MS). Using the α_{1C}-APEX2 and β_{2b}-APEX2 transgenic (TG) mice, 124 proteins were identified with a greater than three-fold increase of normalized spectral counts in the biotinylated samples. Eighty proteins were present in both atria and ventricle datasets. Additional filtering was performed by eliminating proteins such as the Na⁺—Ca²⁺ exchanger and RyR2, as well as numerous kinases, phosphatases and their regulatory subunits, since it was already known that protein kinase A (PKA), PP1 and PP2A regulate Ca_{V}1.2. This left 67 candidates.

The mass spectrometry was performed to identify whether the neighborhood and phosphorylation around Ca_{V}1.2 changes in response to perturbations such as β-adrenergic stimulation. Ca_{V}1.2-APEX2-expressing cardiomyocytes were labeled in the absence or presence of isoproterenol. Biotinylated proteins were enriched by denaturing streptavidin affinity purification, digested into tryptic peptides, labeled with isobaric tandem mass tags (TMT), fractionated by alkaline reversed phase chromatography, and analyzed by triple stage mass spectrometry (TMT SPS MS³). Quantification by SPS MS³, rather than applying conventional tandem mass spectrometry (MS²), has the advantage of reducing signal ratio distortion and enhancing the accuracy of peptide (and, by inference, protein) quantification. Data were collected by an SPS MS³ TMT method using Orbitrap Fusion Lumos mass spectrometers coupled to a Proxeon EASY-nLC 1200 liquid chromatography (LC) system. Peptides were searched using SEQUEST-based in-house software against a mouse proteome database with a target decoy database strategy. Quantitative information on peptides was derived from MS³ scans. A modified version of the Ascore algorithm was used to quantify the confidence assignment of phosphorylation sites.

Of the proteins identified by proximity-labeling in the α_{1C}-APEX2 and β_{2b}-APEX mice, >85% of the proteins were present in both groups using TMT SPS MS³. All 67 potential candidates were included in the datasets acquired by TMT SPS MS³. To be fully inclusive, all proteins identified by both α_{1C}-APEX2 and β_{2b}-APEX2 screens were included, regardless of fold-biotinylation or relative quantification. It was found that 213 proteins in the full ventricular datasets for α_{1C}-APEX and β_{2b}-APEX mice (N>10 replicates) were amongst the 353 proteins previously identified to be significantly phosphorylated in mice upon β₁-adrenergic stimulation. Excluding ion channels, receptors and pumps (e.g. Cacna1c, Cacnb2, Ryr2, Kcnq1, Scn5a, PLN, PLM), kinases and phosphatases, translational machinery, mitochondrial proteins, and proteins already excluded from being potential candidates, 90 candidates remain. Filtering by including only those proteins identified in the atrial screen resulted in a remaining 56 potential candidates, which were considered to be part of the “tier 1” high-priority group. From a dataset of 8,518 phospho-sites of 4,246 proteins, 401 proteins were identified that had phosphorylated Ser or Thr within a consensus PKA phosphorylation site (R/KXS/T, R/KXXS/T, R/KXXXS/T). Cross-referencing these 401 proteins with the atrial and ventricular α_{1C}-APEX2 and β_{2b}-APEX2 datasets yielded 138 proteins, of which 44 were excluded based upon the above criteria. Thus, 55 more proteins were added to the tier 1 high-priority group, including Rrad, a known inhibitor of Ca_{V}1.2. Groups of proteins were selected from this list for the initial studies based upon known t-tubule/membrane localization, and experiments were designed to pare the list.

In addition to the candidate proteins being protein kinase A (PKA) phosphorylation targets, a determination is also made as to whether the interactome and the “neighborhood” changes after β-adrenergic stimulation. Thus, proximity-labeling in cardiomyocytes treated with isoproterenol was compared to untreated controls. Cardiomyocytes were enzymatically isolated from two α_{1C}-APEX2 mice, mixed, loaded with biotin-phenol and exposed to isoproterenol or vehicle for 10 minutes at room temperature. For the final minute, labeling was initiated with H₂O₂. This was repeated for myocytes from 4 additional sets of mice (total: 5 sets of biological replicates; 10 mice). Biotinylated proteins were purified and detected using TMT SPS MS³. The isobaric labeling allowed simultaneous quantification of all proteins measured rather than just a select subset, thus enabling statistical analysis of relative enrichment between vehicle and isoproterenol treatment. Candidates in the upper left quadrant have reduced labeling (likely indicating reduced proximity) after isoproterenol and candidates in the upper right quadrant have increased labeling (likely indicating increased proximity) after isoproterenol. Mitochondrial/nuclear proteins were removed from the analysis. The labeling of the catalytic subunit of PKA (Prkaca) increased. Other significant changes were the ˜50% decrease in Rad (Rrad) (P=6.9×10⁴) and ˜57% reduction in Sorbin and SH3 domain containing 2 (Arg/Abl-Interacting Protein 2; Sorbs2) (P=0.004). Similar experiments for β₂-APEX, and atrial myocytes for both α_{1C}-APEX2 and β₂-APEX2 are also contemplated, and more “hits” are anticipated as the sample size increases. It is important to note, however, that changes in proximity do not necessarily imply changes in interactions with Ca_{V}1.2.

Mass spectrometry identified phosphorylated proteins from biotinylated, streptavidin-purified samples from APEX2 mice, and from whole hearts of wild-type (WT) mice treated with isoproterenol. Both Rad and Sorb2 are phosphorylated on multiple residues. Rad, a member of the RGK family of GTP-binding proteins, is an inhibitor of voltage-gated Ca²⁺ channels and is also a PKA target. Although RGK proteins do not contain canonical lipid modification motifs, they are enriched at the plasma membrane, although some RGK proteins are also localized to the nucleus, cytosol and actin/microtubule network. To further explore whether β-agonists can alter membrane-localization of Rad relative, rat cardiomyocytes were infected with adenoviruses harboring cyan fluorescent protein (CFP)-tagged Rad. The fluorescence of CFP-Rad at the membrane and in the cytosol was quantified before and after isoproterenol. The number of exposures were limited to avoid photo-bleaching. It was found that isoproterenol decreased Rad localized at the membrane by 21%±3%, whereas cytosolic Rad increased by 20%±5% (P<0.0001, as shown in FIG. 4). These data suggest that the proximity of Rad relative to Ca_{V}1.2 decreases after isoproterenol, perhaps due to phosphorylation of Rad. Deletion of Rad increases baseline Ca²⁺ current, thereby reducing any effect of β-adrenergic regulation of Ca_{V}1.2, but its role as the PKA target in mediating β-adrenergic regulation of Ca_{V}1.2 has not been explored, although over-expression of Rad in cardiomyocytes inhibits forskolin-induced Ca²⁺ current stimulation.

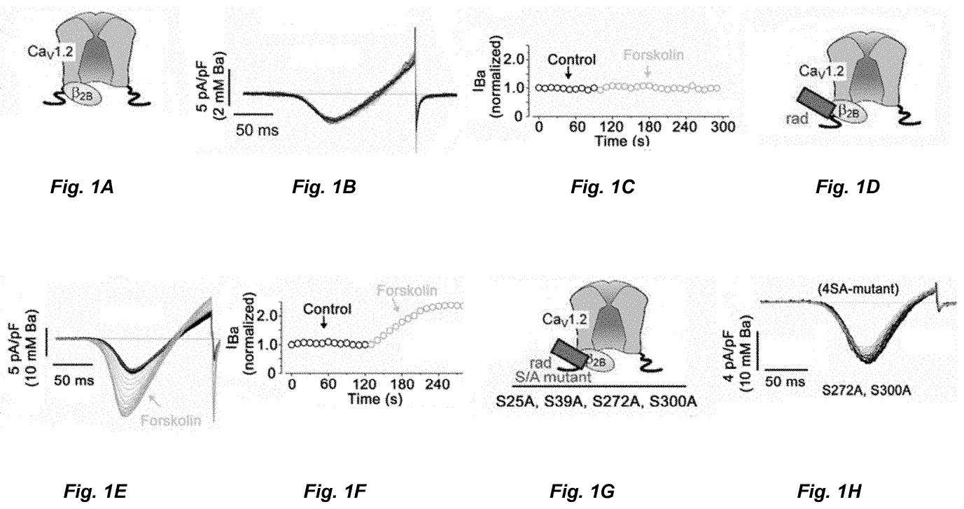

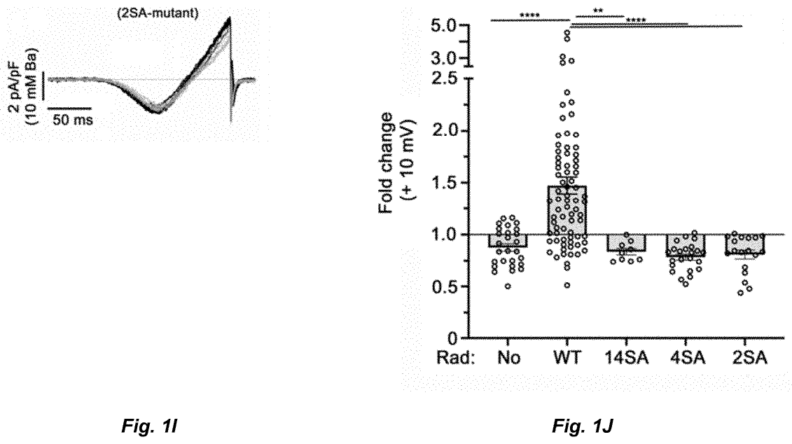

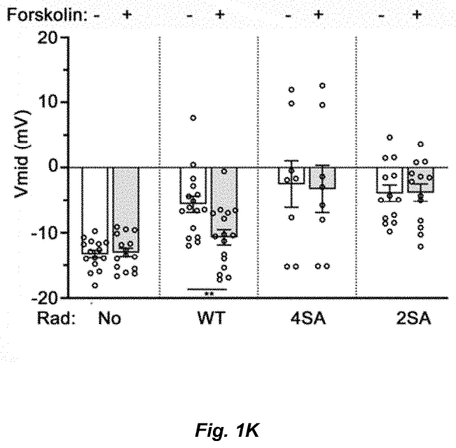

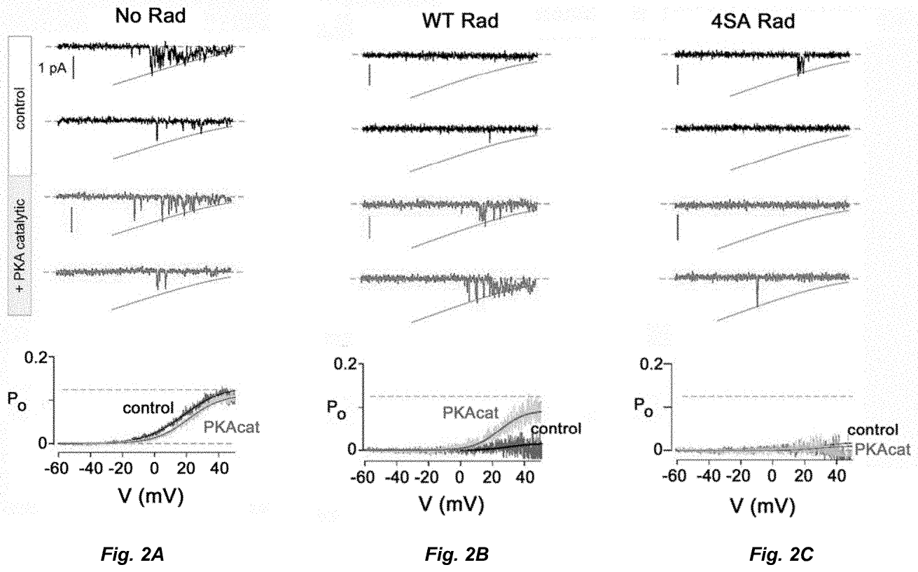

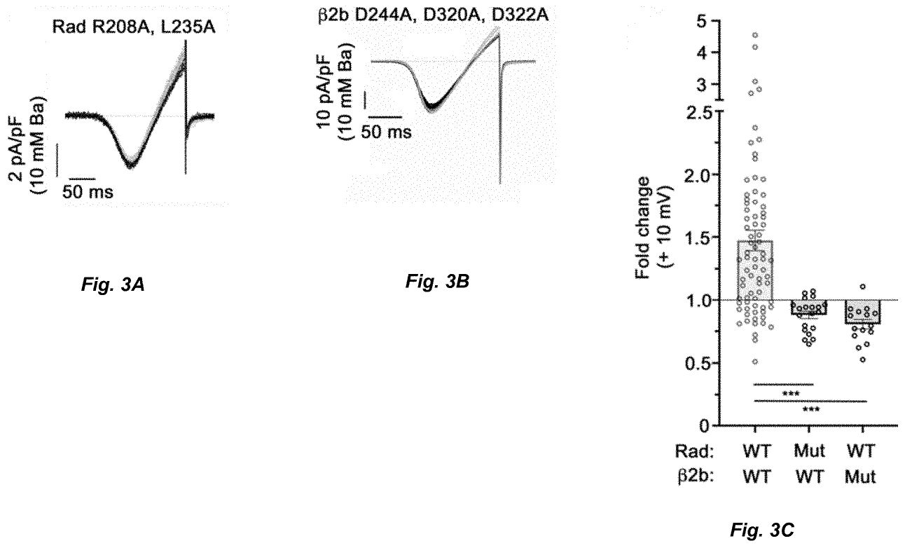

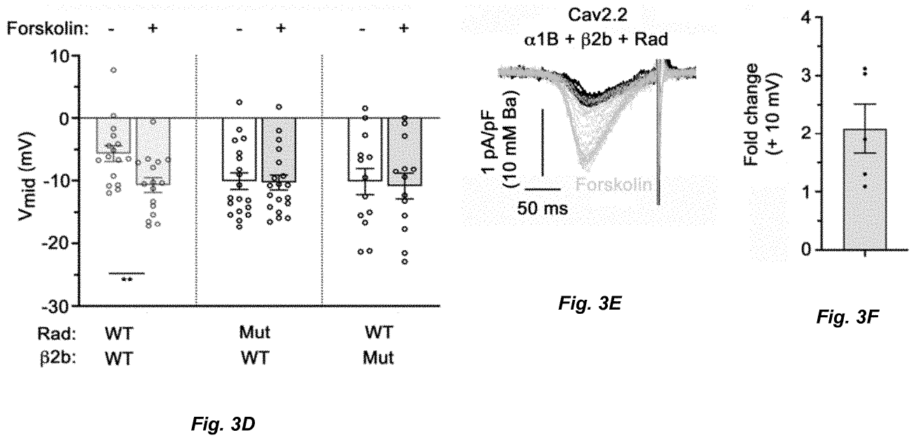

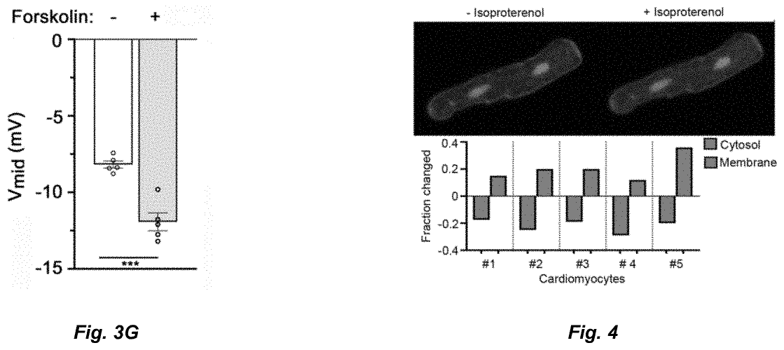

FIG. 1(d,e,f,k) shows that the addition of Rad to α_{1C} and β₂ results in baseline inhibition of the channel, and that this inhibition can be reversed via addition of forskolin, which causes phosphorylation of Rad. FIGS. 1G, 1H and 1I show the addition of SA mutant Rad. As illustrated in FIGS. 2A, 2B and 2C, expression of PKA catalytic subunit with WT Rad, but not 4SA-mutant Rad, potently up-regulates the Pₒ of Ca_{V}1.2. These data demonstrate that Rad inhibits single α_{1C} channels at baseline and that phosphorylation of Rad results in disinhibition of the channel and increased channel current. FIGS. 3A-3G demonstrate that mutating Rad or β2 in a manner that prevents the two from interacting with one another results in channels that cannot be inhibited by Rad, because these channels are not inhibited at baseline. Therefore, these channels do not exhibit an increased current in response to forskolin.

Increased cardiac contractility during fight-or-flight response is caused by (3-adrenergic augmentation of Ca_{V}1.2 channels. In transgenic murine hearts expressing fully PKA phosphorylation-site-deficient mutant Ca_{V}1.2 α_{1C} and β subunits, this regulation persists, implying involvement of extra-channel factors. Here, the inventors identified the mechanism by which β-adrenergic agonists stimulate voltage-gated Ca²⁺ channels. The inventors expressed ascorbate-peroxidase-conjugated-α_{1C} or β_{2B} subunits in mouse hearts and used multiplexed, quantitative proteomics to track hundreds of proteins in proximity of Ca_{V}1.2. The inventors observed that Rad is enriched in the Ca_{V}1.2 micro-environment but is depleted during β-adrenergic stimulation. The inventors found that PKA-catalyzed phosphorylation of Rad relieves its inhibition of Ca_{V}1.2 observed as an increase in channel open probability that depended on specific Ser residues in Rad. Expression of Rad or Rem, another member of this small G-protein family, also imparted PKA-induced stimulation of Ca_{V}1.3 and Ca_{V}2.2. These results reveal an evolutionary conserved mechanism that confers adrenergic-modulation to voltage-gated Ca²⁺ channels.

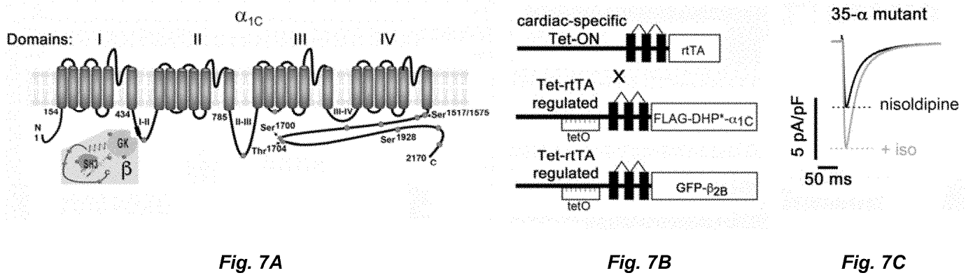

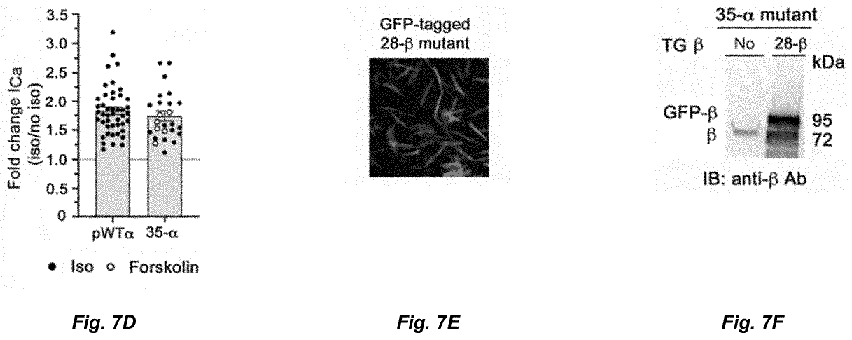

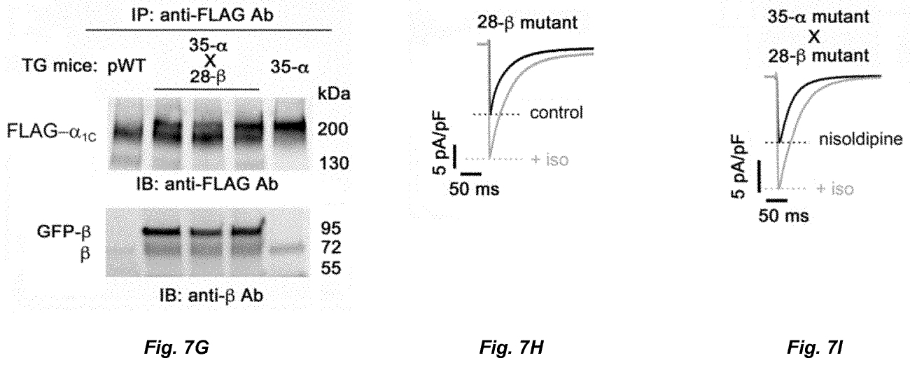

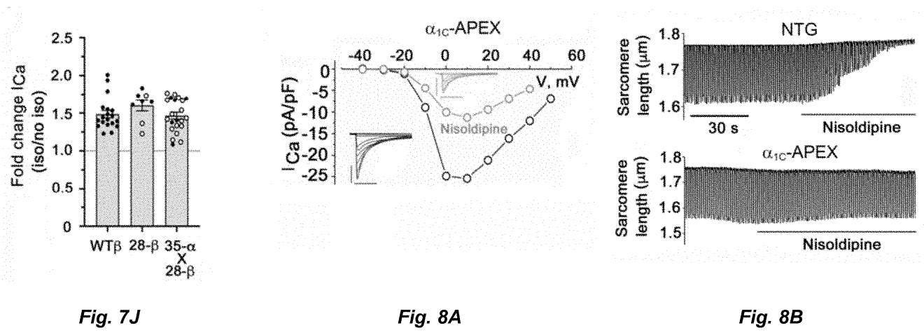

The positive inotropic effect of β-adrenergic agonists on the heart is a classical physiological phenomenon universally experienced during excitement, exercise, fight or flight. This effect is mediated by β-adrenergic activation of protein kinase A (PKA) which leads to increased Ca²⁺ influx through L-type Ca_{V}1.2 channels in cardiomyocytes. The molecular mechanism of PKA-induced up-regulation of Ca_{V}1.2 current (I_{Ca,L}) (FIG. 7A) has been intensively studied for many decades without resolution. The commonly accepted model is that PKA increases Ca_{V}1.2 current by phosphorylating Ca_{V}1.2 α_{1C}- and/or β_{2B}-subunits. However, the proposed regulatory residues on the C-termini of α_{1C} (Ser¹⁹²⁸; Ser/Thr^{1700/1704}) and β_{2B} (Ser⁵¹² and Ser⁵⁷⁰) in cardiomyocytes were shown to be inessential for β-adrenergic stimulation of Ca²⁺ currents in the heart. Nevertheless, given the multiple other Ser/Thr residues on α_{1C} and β_{2B}, it remained possible that PKA phosphorylation of some combination of these was responsible for β-adrenergic modulation of Ca_{V}1.2 in cardiomyocytes. As shown below, this also is not the case.

FIG. 7A schematically illustrates rabbit cardiac α_{1C} and β subunits. Red dots indicate putative PKA phosphorylation sites. FIG. 7B schematically illustrates the binary transgene system. The expression of reverse tetracycline-controlled transactivator (rtTA) is driven by the cardiac-specific α-myosin heavy chain promoter. The cDNAs for FLAG-DHP-resistant α_{1C} or GFP-β_{2B} were ligated behind a minimal cytomegalovirus (CMV) promoter and 7 tandem tetO sequences. This Tet-ON system requires doxycycline for binding to and inducing a Tet-responsive promoter. FIG. 7C is an exemplary graph showing whole-cell Ca_{V}1.2 currents of 35-mutant α_{1C} transgenic mice cardiomyocytes. Pulses from −50 mV to 0 mV in 300 nM nisoldipine (black trace) and after 200 nM isoproterenol (iso) in presence of nisoldipine (blue trace). FIG. 7D is a graph showing fold change (iso vs. no iso) of peak Ca²⁺ current (I_{Ca}) at 0 mV caused by isoproterenol (closed circle) or forskolin (open circle) for pWT α_{1C} and 35-mutant α_{1C} channels. Mean±SEM. P=0.81 by t-test. FIG. 7E is a fluorescent image of isolated cardiomyocytes expressing GFP-tagged 28-β mutant. FIG. 7F shows an anti-13 antibody immunoblot of cleared lysates from doxycycline-fed 35-mutant α_{1C} transgenic mice or 35-mutant α_{1C} X GFP-tagged 28-mutant β_{2B} expressing mice hearts. FIG. 7G shows anti-FLAG antibody (upper) and anti-13 antibody (lower) immunoblots of anti-FLAG antibody immunoprecipitations from cleared lysates of hearts from pWT, 35-α and 35-α X GFP-tagged-28-β expressing mice. FIG. 7H is a graph showing exemplary whole-cell Ca_{V}1.2 currents of GFP-tagged-28-mutant β_{2B} transgenic mice cardiomyocytes. Pulses from −50 mV to 0 mV before (black trace) and after addition of 200 nM isoproterenol (blue trace). FIG. 7I is a graph showing exemplary whole-cell Ca_{V}1.2 currents recorded from 35-mutant α_{1C} X 28-mutant β_{2B} transgenic mice cardiomyocytes. Pulses from −50 mV to 0 mV in 300 nM nisoldipine (black trace) and after 200 nM isoproterenol in presence of nisoldipine (blue trace). FIG. 7J is a graph showing the fold change (iso vs. no iso) at 0 mV in peak Ca²⁺ current caused by isoproterenol (closed circle) or forskolin (open circle) for cardiomyocytes isolated from transgenic mice expressing GFP-tagged WT β_{2B} subunit, GFP-tagged 28-mutant β_{2B}, or both 35-mutant α_{1C} and GFP-tagged 28-mutant β_{2B}. Mean±SEM. P=0.86 by one way-ANOVA.

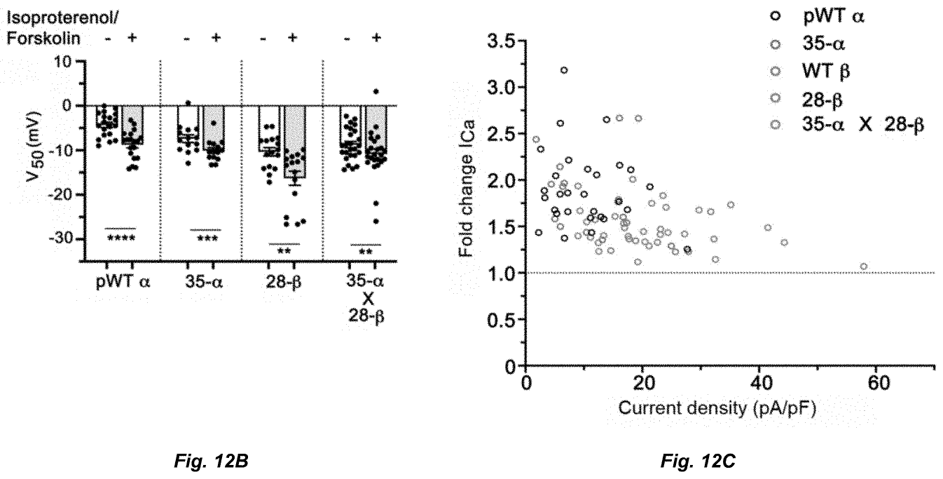

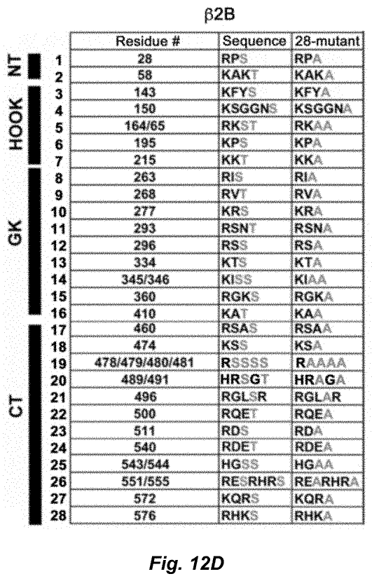

Phosphorylation of core Ca_{V}1.2 channel subunits is not required for acute β-adrenergic regulation. The inventors developed a transgenic approach that enables doxycycline-inducible expression in mice of FLAG-epitope tagged, dihydropyridine (DHP)-resistant Ca_{V}1.2 channels (FIG. 7B). Transgenic DHP-resistant Ca²⁺ current is distinguishable from endogenous Ca_{V}1.2 current by the application of nisoldipine, a Ca^{2±}-channel DHP-antagonist. All 51 of both conserved and non-conserved Ser and Thr within the 35 intracellular PKA consensus phosphorylation sites of rabbit α_{1C} were mutated to Ala (construct termed “35-mutant α_{1C}”; see FIG. 12A). Cardiomyocytes from transgenic mice expressing DHP-resistant 35-mutant α_{1C} displayed nisoldipine-insensitive current. This was both up-regulated and activated at more negative potentials by isoproterenol, a non-selective β-adrenoreceptor agonist, and by forskolin, an activator of adenylyl cyclase. The 35-mutant α_{1C} channels were activated to the same extent as were the pseudo-WT (pWT) α_{1C} channels (FIGS. 7C and 7D; and FIGS. 12B and 12C).

All 37 conserved and non-conserved Ser and Thr within 28 PKA-consensus phosphorylation sites of human β_{2B} were mutated to Ala (construct termed “28-mutant β_{2B}”; see FIG. 12D). As above, cardiomyocytes isolated from transgenic mice expressing doxycycline-inducible GFP-tagged 28-mutant β_{2B} (FIGS. 7E-7G) displayed isoproterenol or forskolin-induced stimulation of Ca_{V}1.2 current amplitude (FIGS. 7H and 7J), and a hyperpolarizing shift in the voltage-dependence of activation (FIG. 12B), similar to cardiomyocytes isolated from transgenic mice expressing GFP-tagged WT β_{2B}.

Finally, the inventors crossed the 35-mutant α_{1C} transgenic mice with the 28-mutant β_{2B} transgenic mice. Feeding these mice doxycycline overnight induced robust expression of both 35-mutant α_{1C} and GFP-tagged 28-mutant β_{2B} in the heart, and anti-FLAG antibody immunoprecipitation indicated GFP-tagged 28-mutant β_{2B} subunits dominate in the 35-mutant α_{1C} complex (FIG. 7G). These mutant channels also displayed a normal isoproterenol- or forskolin-induced increase in peak Ca²⁺ current (FIGS. 7I and 7J) and a hyperpolarizing shift in the V₅₀ of activation (FIG. 12B). These results indicate that β-adrenergic-stimulation of Ca_{V}1.2 does not involve direct phosphorylation of α_{1C} or β₂ subunits.

FIG. 12A is a table showing the 35 putative PKA phosphorylation sites in rabbit α_{1C}. The 51 residues in red are either predicted phosphorylation sites or within the immediate region of the predicted phosphorylation sites. All 51 residues were replaced with Ala in the 35-mutant α_{1C} transgenic mice. FIG. 12B is a combined bar and column scatter plot of Boltzmann function parameters V₅₀. ** p<0.01; *** p<0.001; **** p<0.0001 by paired t-test. pWT α, n=19; 35-α mutant, n=14; 28-β mutant, n=16; 35-α mutant X 28-β mutant, n=15. FIG. 12C is a graph of isoproterenol and forskolin-induced increase in nisoldipine-resistant current stratified by total basal current density before nisoldipine. FIG. 12D is a table showing the 28 putative PKA phosphorylation sites in the N-terminal (NT), Hook, GK and C-terminal (CT) domains of β_{2B}. The 37 residues in red are either predicted phosphorylation sites or within the immediate vicinity of predicted phosphorylation sites, and were mutated to Ala in the 28-mutant GFP-tagged β_{2B} transgenic mice.

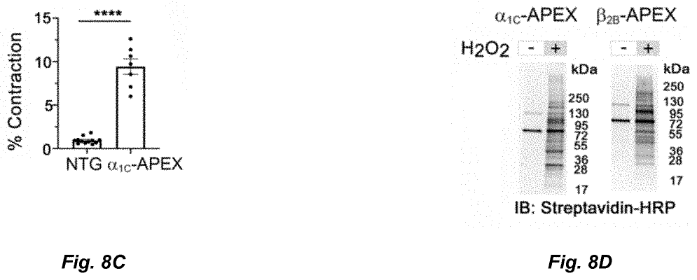

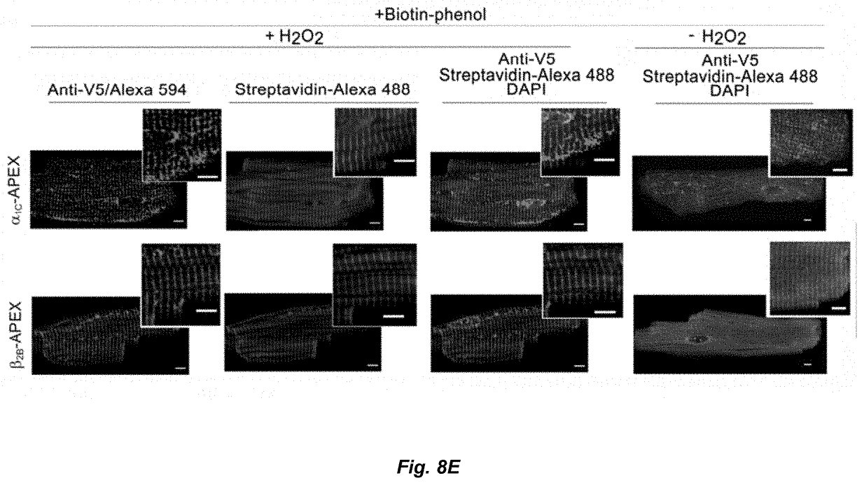

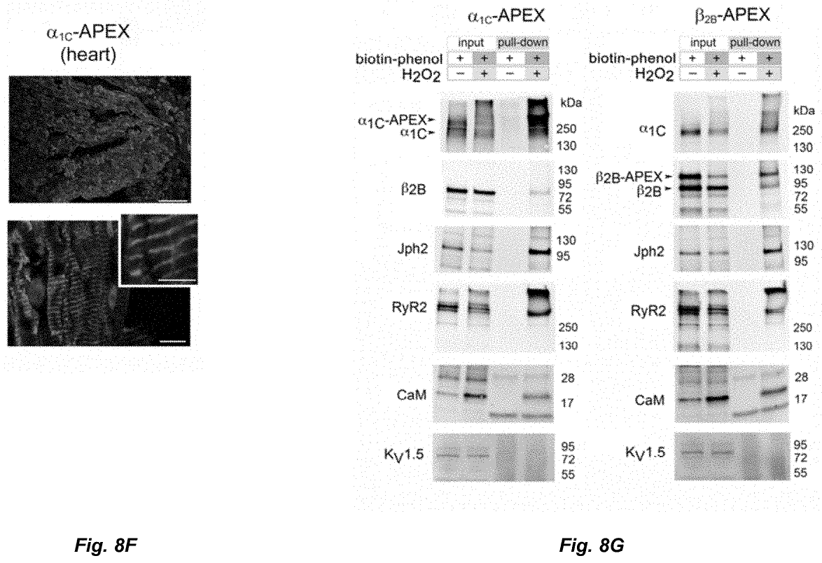

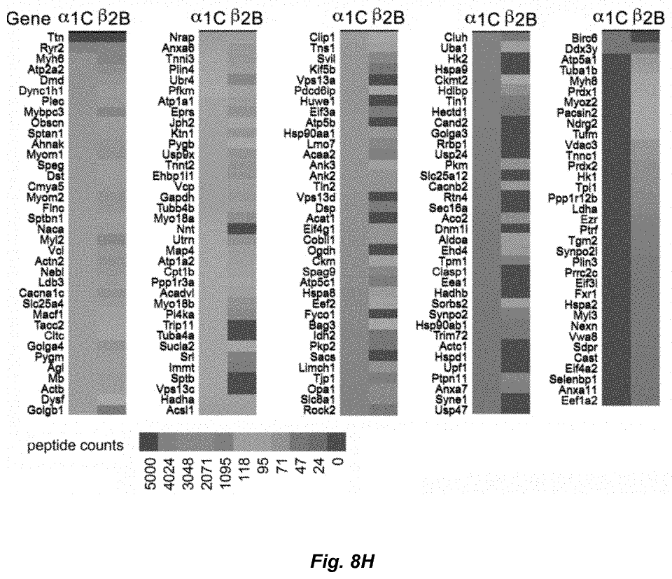

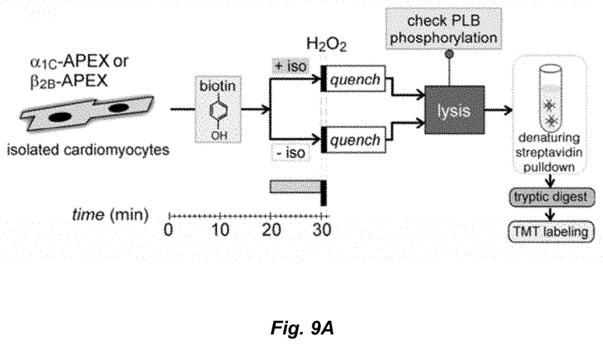

A peroxidase-catalyzed proximity labeling system in cardiomyocytes and intact hearts reveals the Ca_{V}1.2 proteome subdomain. Given the foregoing results, it seemed likely that β-adrenergic stimulation of Ca²⁺ current in the heart involves recruitment of an activator protein to or loss of an inhibitory protein from the Ca_{V}1.2 macromolecular complex. Accordingly, the inventors adapted for application to cardiomyocytes an enzyme-catalyzed proximity labeling method originally developed for the identification of the mitochondrial proteome. The inventors generated transgenic mice with doxycycline-inducible, cardiomyocyte-specific expression of V5-tagged-APEX2-DHP-resistant-α_{1C} or V5-tagged-APEX2-β_{2B} proteins. These resulted in in situ biotin-labeling and subsequent identification by mass spectrometry of Ca_{V}1.2 near-neighbors. Importantly, fusing APEX2 to α_{1C} and β_{2B} did not affect Ca_{V}1.2 subcellular localization and function in cardiomyocytes because: 1) APEX2-conjugated α_{1C} subunits inserted in the membrane and were appropriately activated by voltage (FIG. 8A) field-stimulation in the presence of 300 nM nisoldipine of cardiomyocytes isolated from transgenic DHP-resistant APEX2-α_{1C} mice induced contraction (FIGS. 8B and 8C).

FIG. 8A is a graph showing exemplary current-voltage relationship of Ca²⁺ currents from α_{1C}-APEX2 mice cardiomyocytes acquired in the absence (black trace) and presence of 300 nM nisoldipine (red trace). Inset scale bars: horizontal 100 ms, vertical 10 pA/pF. FIG. 8B shows the time course of changes in sarcomere length after superfusion of nisoldipine (300 nM) containing solution. Cardiomyocytes were field-stimulated at 1-Hz. FIG. 8C is a bar graph of percent (%) shortening in the absence and presence of nisoldipine. Mean±SEM. ****, P<0.0001 by t-test. FIG. 8D shows a streptavidin-HRP blot of lysates of isolated ventricular cardiomyocytes from α_{1C}-APEX2 and β_{2B}-APEX2 transgenic mice. FIG. 8E shows the anti-V5 antibody/Alexa 594 fluorescence and streptavidin-Alexa 488 fluorescence of cardiomyocytes isolated from α_{1C}-APEX2 and β_{2B}-APEX2 expressing mice exposed to biotin-phenol and H₂O₂ or no H₂O₂. Nuclear labeling with DAPI stain. Images obtained with confocal microscopy. Scale bar=5 μm. FIG. 8F shows the streptavidin-Alexa 488 fluorescence of tissue sections of Langendorff-perfused α_{1C}-APEX2 heart. Nuclear labeling with DAPI stain. Images obtained with confocal microscopy. Scale bars: uppert—100 μm; lower—10 μm; lower inset—20 μm. FIG. 8G shows immunoblots of biotin-labeled proteins from α_{1C}-APEX2 and β_{2B}-APEX2 mice cardiomyocytes. In contrast to CaV1.2 subunits, RyR2, Jph2 and CaM, K_{V}1.5 channels were not biotinylated. FIG. 8H shows proteins with a ratio of >2 (measured by normalized TMT signal/noise) in the experimental conditions compared to a no-labeling control (no H₂O₂) are sorted by spectral counts. The 150 proteins with the highest peptide counts are displayed in the color-coded table. α_{1C}-APEX2 and β_{2B}-APEX2 data were collected in biological duplicate experiments.

The inventors initiated proximity labeling in either isolated ventricular cardiomyocytes or Langendorff-perfused whole hearts by incubating or perfusing with a solution containing biotin-phenol (biotin-tyramide) followed by exposure to H₂O₂. The short half-life (1-2 msec) of enzyme-generated biotin-phenoxyl radicals ensures that labeling of proteins occurs only within ˜20 nm of the enzyme. The distance between the sarcoplasmic reticulum membrane and the sarcolemma at the dyad is 12-15 nm. Western blots of extracted proteins probed with streptavidin-HRP confirmed robust biotinylation of proteins in cardiomyocytes isolated from both α_{1C}-APEX2 and β_{2B}-APEX2 transgenic mice (FIG. 8D). Immunofluorescence with anti-V5 antibody showed transgenic Ca_{V}1.2, similar to endogenous channels were targeted to the transverse tubules (FIG. 8E). Consistent with the specific localization and the short range of labeling, the inventors visualized biotin-labeled proteins with streptavidin-FITC in a striated z-disk pattern that coincided with the pattern of transgenic α_{1C} and β_{2B} subunits visualized by anti-V5 antibody immunofluorescence (FIGS. 8E and 8F).

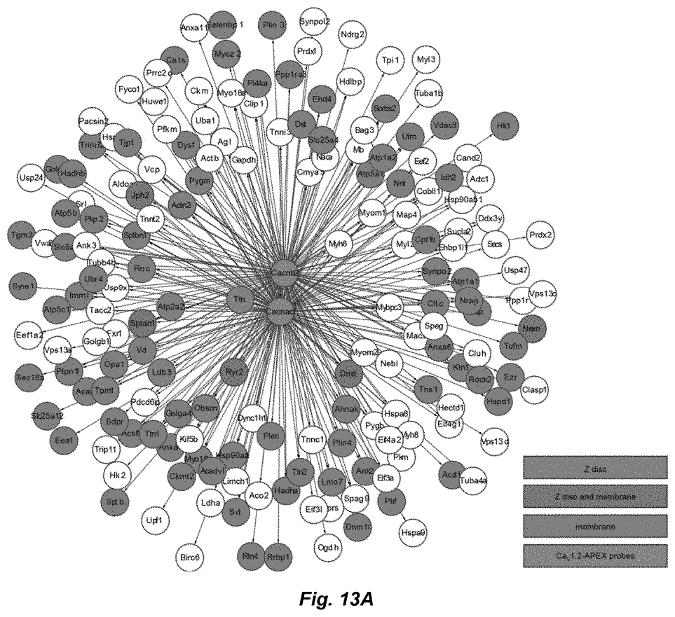

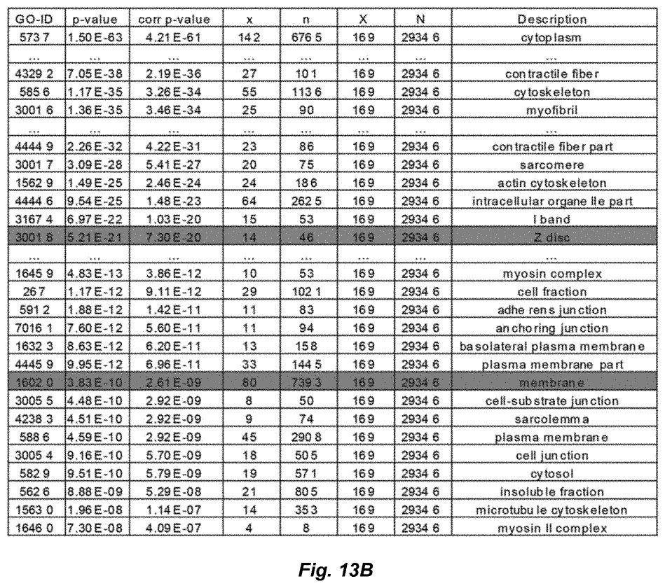

The inventors extracted and isolated the biotinylated proteins by affinity purification on streptavidin beads of whole-cell lysates. Purified proteins were analyzed either by Western blotting (FIG. 8G) to probe a limited set of known proteins or by triple-stage mass spectrometry (TMT SPS MS³) (FIG. 8H) for an unbiased and much more comprehensive analysis. The Western blots demonstrated the biotinylation of proteins known to be present together with Ca_{V}1.2 at dyadic junctions in cardiomyocytes, namely ryanodine receptors (RyR2), calmodulin (CaM), and junctophilin (Jph2) (FIG. 8G). The repolarizing K_{V}1.5 channels were not biotinylated (FIG. 8G), in contrast, implying that these channels may not be closely localized to Ca_{V}1.2. In addition to these, the TMT SPS MS³ approach identified hundreds of other proteins close to Ca_{V}1.2 (FIG. 8H), although many are likely bystanders rather than interacting proteins. Of these, the 150 proteins with the highest peptide-counts were remarkably similar for the α_{1C}-APEX2 and β_{2B}-APEX2 transgenic mice. These were primarily classified as being membrane, cytoskeletal, and sarcomeric proteins (FIGS. 13A and 13B). Some of these proteins, however, were associated with other compartments including ribosomes, endoplasmic reticulum, Golgi, and endosomes. The source of this labeling was likely Ca_{V}1.2-APEX2 during its synthesis and translocation.

FIG. 13A illustrates the prefuse force directed map of proteins from FIG. 8H. Peptide counts were used as weight. Proteins mapping to the GO-terms “Z disc” are colored in green, to “membrane” in yellow and to both in purple. The α_{1C}-APEX2 and β_{2B}-APEX2 are colored in blue. FIG. 13B is a table showing the GO-term (cellular localization) enrichment for proteins in FIG. 8H.

With regard to isoproterenol-induced changes in the Ca_{V}1.2 neighbors, recently, APEX-labeling, combined with either multiplexed quantitative mass spectrometry or quantitative proteomics using a system of spatial references and bystander ratio calculations, was utilized to analyze ligand-induced changes in the local environment of G-protein-coupled receptors. Similarly, by identifying proteins the proximity-labeling of which change in response to β-adrenergic stimulation, the inventors reasoned that the hypothesized unknown PKA target involved in up-regulating Ca_{V}1.2 current could be identified.

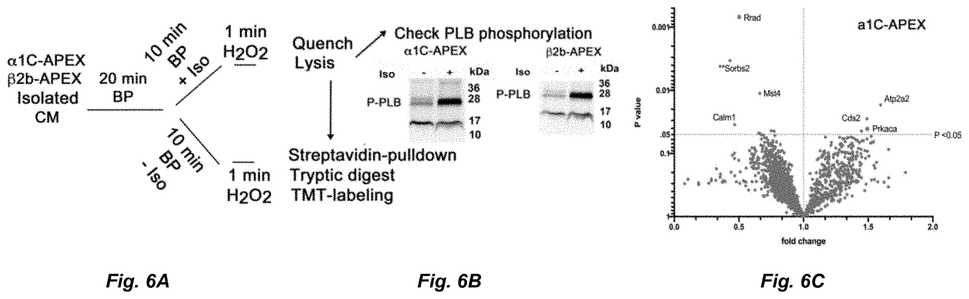

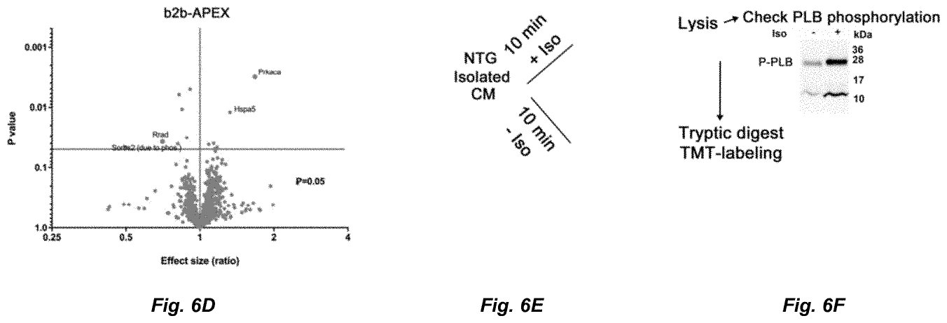

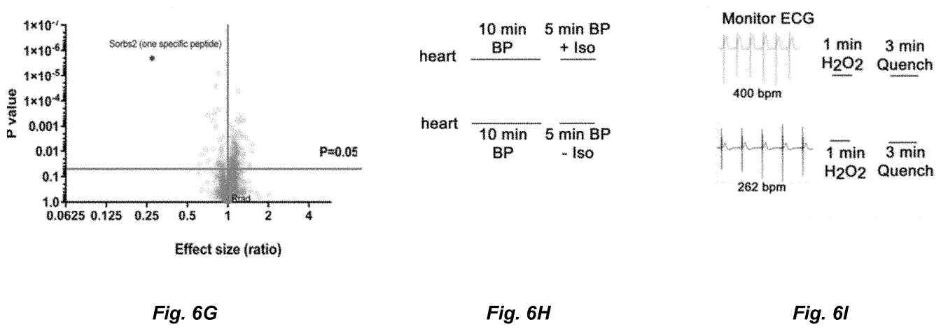

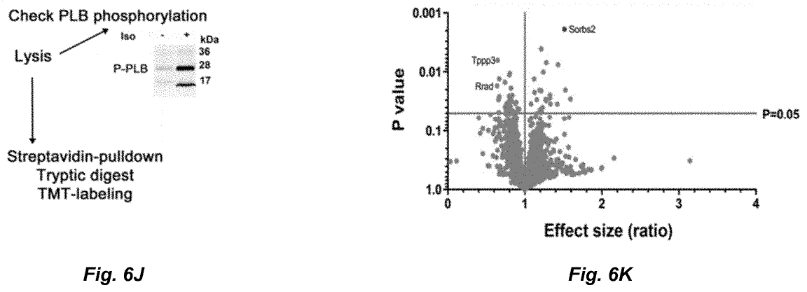

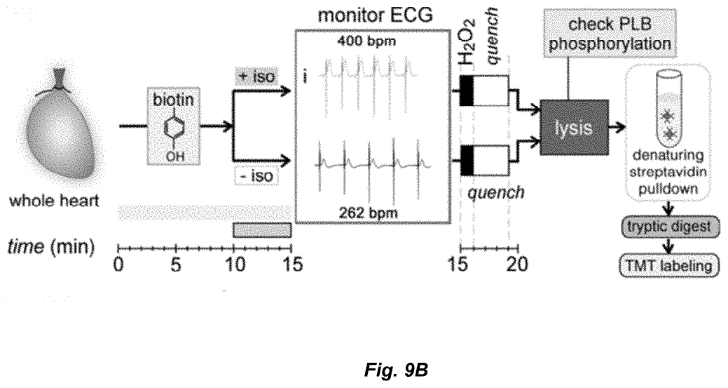

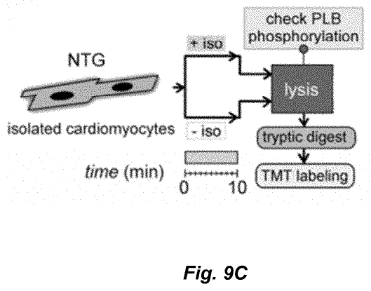

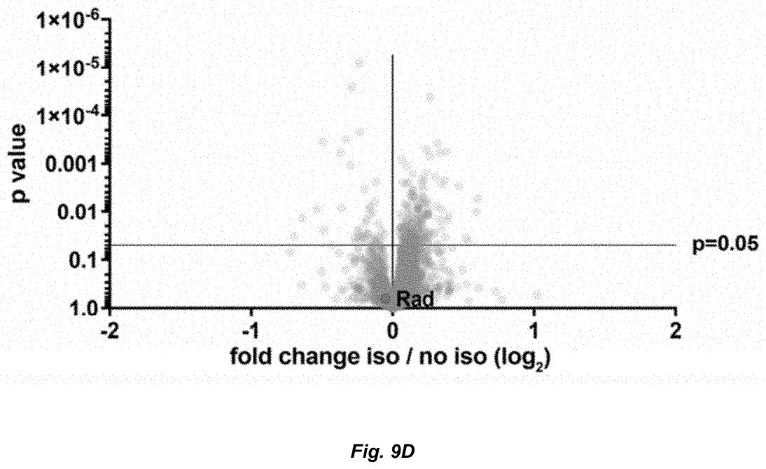

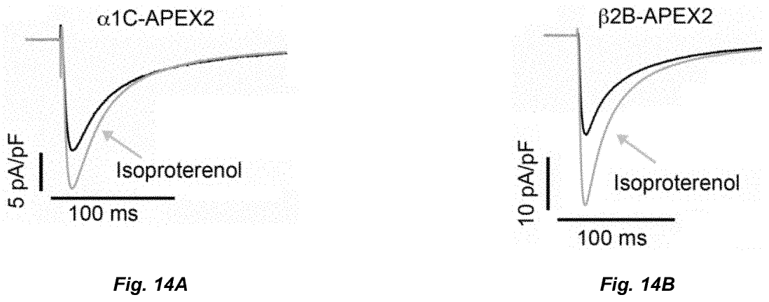

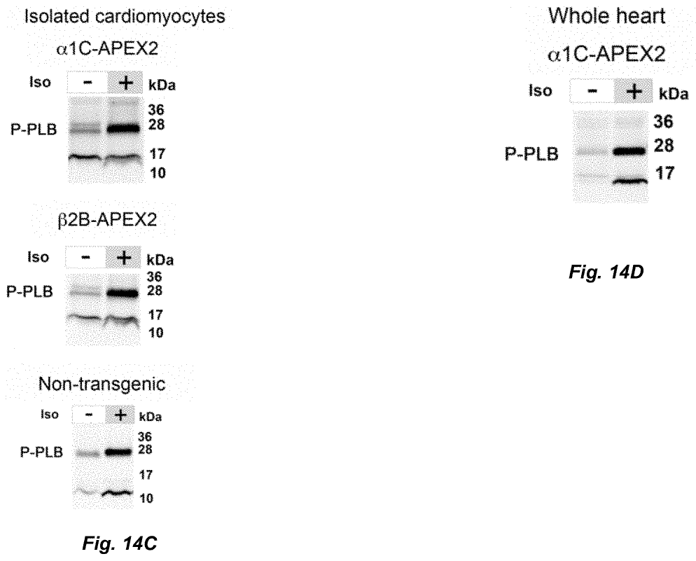

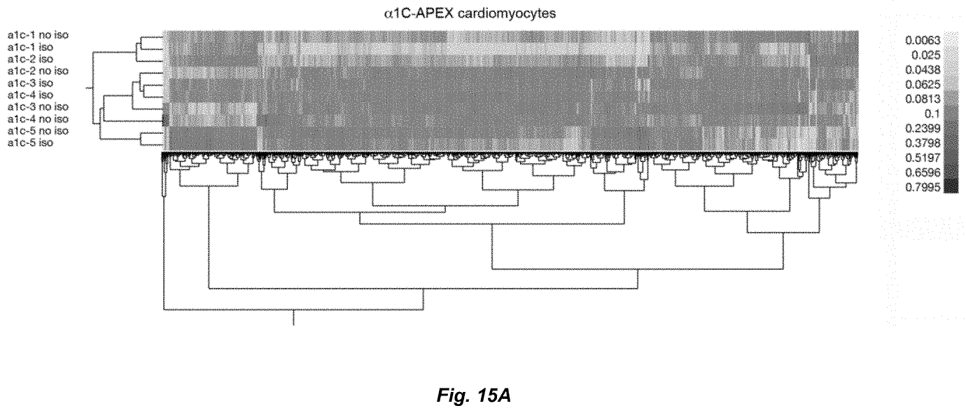

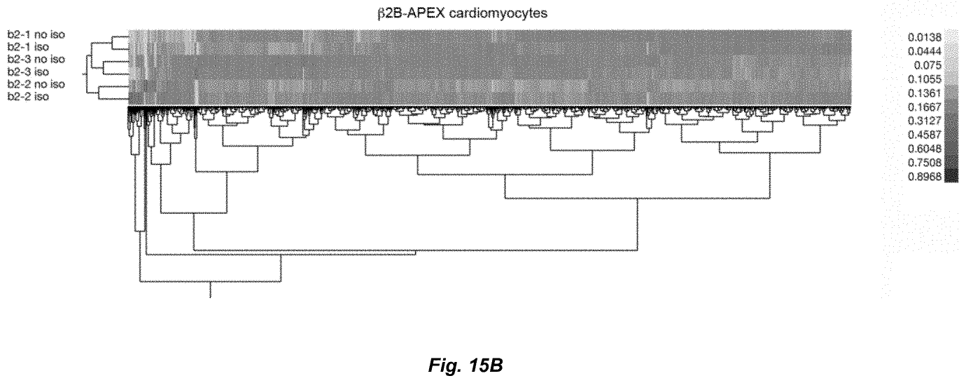

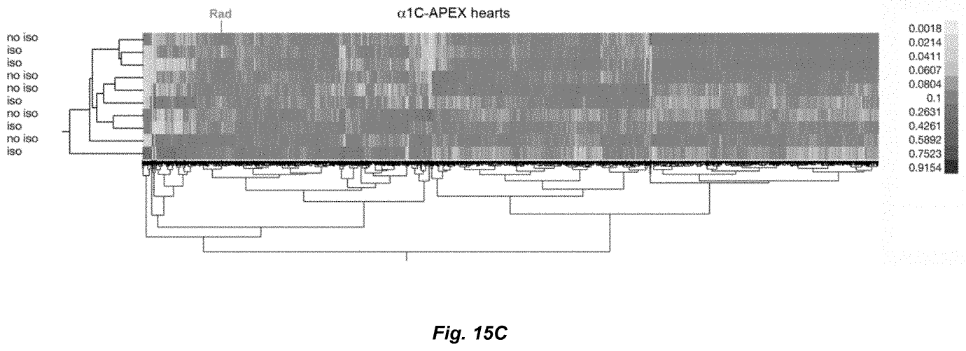

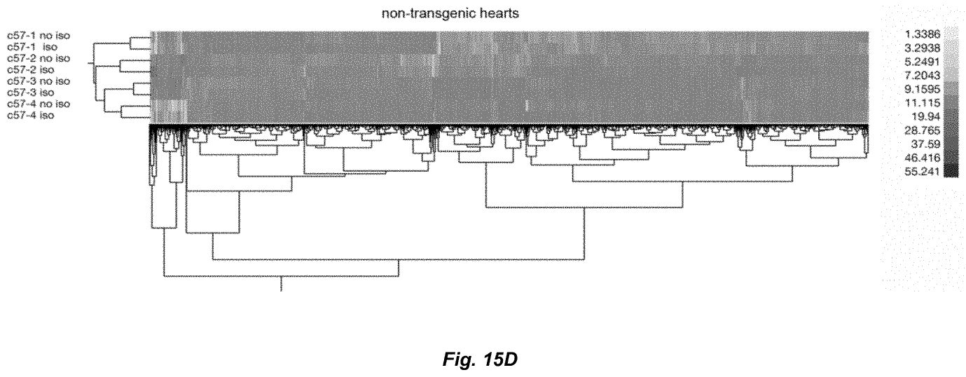

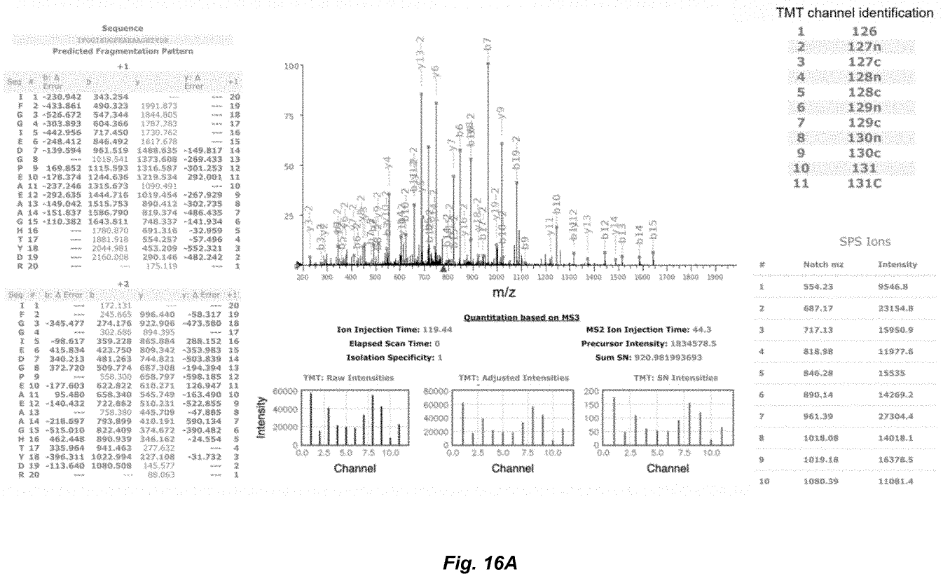

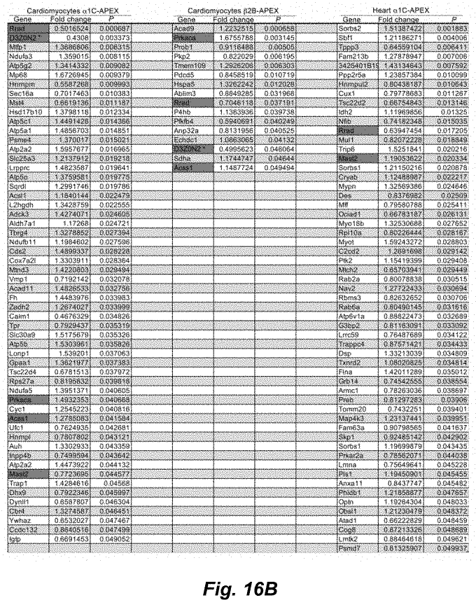

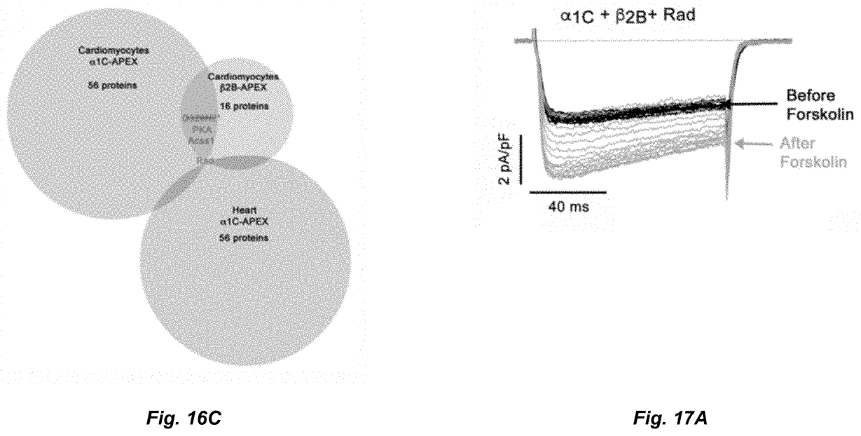

The nisoldipine-resistant Ca²⁺ currents from α_{1C}-APEX2 mice and the Ca_{V}1.2 current from β_{2B}-APEX2 mice are stimulated by isoproterenol (FIGS. 14A and 14B). Thus, conjugation of the peroxidase to the Ca_{V}1.2 subunits does not interfere with its (3-adrenergic regulation. Isolated cardiomyocytes were pre-incubated with biotin-phenol for 30-minutes, and during the final 10-minutes, also exposed either to isoproterenol or to vehicle (FIG. 9A). Labeling was initiated with H₂O₂ for one-minute. Biotinylated proteins were purified and quantified using TMT SPS MS³, with the analyses tracking 1951 proteins for quintuplicate biological replicates of α_{1C}-APEX2 (FIG. 6C and FIG. 15A) and 1936 proteins for triplicate biological replicates of β_{2B}-APEX2 cardiomyocytes (FIG. 6D and FIG. 15B). The inventors also probed the impact of isoproterenol on the Ca_{V}1.2 proteome signature in Langendorff-perfused whole hearts (FIG. 9B), tracking 2,610 proteins from 10 hearts in parallel (FIG. 6K, FIG. 15C and FIG. 16A). The inventors monitored phospholamban phosphorylation to confirm that the β-adrenergic signaling pathway in cardiomyocytes is preserved in the presence of biotin-phenol and H₂O₂ (FIG. 14C and FIG. 14D). The relative summed peptide TMT signal-to-noise, as used for relative protein quantification, dropped for several proteins. Notably, all three approaches indicated a 30-50% decrease in the amount of the small Ras-like G-protein, Rad, following application of isoproterenol (FIGS. 6C, 6D and 6K). Analyzing the overlap of proteins displaying isoproterenol-induced changes in α_{1C}-APEX2 and β_{2B}-APEX2 experiments, Rad was the only candidate protein displaying this behavior (FIG. 16B and FIG. 16C). In contrast, a 10-minute exposure of cardiomyocytes isolated from non-transgenic mice to isoproterenol (FIG. 9C) had a minimal effect on peptides relatively quantified by TMT SPS MS³ and specifically no significant effect on the amount of Rad as compared to non-treated paired cardiomyocytes (FIG. 9D and FIG. 15D). Of course, these latter peptides mapping to Rad were from the general population and were not proximity labeled, indicating that the overall level of Rad in cardiomyocytes did not change by isoproterenol stimulation. In other words, Rad is depleted by β-adrenergic stimulation from the neighborhood of Ca_{V}1.2, not from the cell as a whole.

FIG. 9A schematically shows protein labeling and workflow for isolated cardiomyocytes (CM). Iso=1 μM isoproterenol. PLB=phospholamban. FIG. 6C is a volcano plot of fold-change of iso vs. no iso for relative protein quantification by TMT mass spectrometry of α_{1C}-APEX2 samples. Data shown are means for 5 pairs of samples. P-values were false discovery rate (FDR) corrected. Rad (red dot) is reduced by 50% and PKA catalytic subunit (green dot) is increased by 50%. FIG. 6D is similar to that shown in FIG. 6C, except using cardiomyocytes from β_{2B}-APEX2 mice. Data shown are means for 3 pairs of samples. Rad (red dot) is reduced by 30% and PKA catalytic subunit (green dot) is increased by 68%. FIG. 9B schematically illustrates protein labeling and workflow for Langendorff-perfused α_{1C}-APEX2 mice hearts. Electrocardiogram confirmed response to isoproterenol. BPM=beats per minute. FIG. 6K is a volcano plot of fold-change of iso vs. no iso for relative protein quantification by TMT mass spectrometry of α_{1C}-APEX2 whole heart samples. Data shown are means for 10 hearts, 5 without isoproterenol and 5 with isoproterenol. P values were FDR corrected. Rad (red dot) is reduced by 36%. FIG. 9C schematically illustrates protein labeling and workflow for isolated cardiomyocytes from non-transgenic (NTG) mice. FIG. 9D is a volcano plot of fold-change of iso vs. no iso for relative protein quantification by TMT mass spectrometry of proteins isolated from non-transgenic (NTG) mice cardiomyocytes. Data shown are means for 4 pairs of samples. P-values are FDR corrected. Rad (red dot) in whole heart is unchanged by isoproterenol.

FIG. 14A is an exemplary graph showing whole-cell Ca_{V}1.2 currents recorded from freshly dissociated cardiomyocytes of α_{1C}-APEX2 transgenic mice. Pulses from −50 mV to 0 mV in 300 nM nisoldipine (black trace) and after 200 nM isoproterenol in presence of nisoldipine (blue trace). FIG. 14B is an exemplary graph showing whole-cell Ca_{V}1.2 currents recorded from freshly dissociated cardiomyocytes of β_{2B}-APEX2 transgenic mice. Pulses from −50 mV to 0 mV, before (black trace) and after 200 nM isoproterenol (blue trace). FIG. 14C shows Western blots probed with phospho-phospholamban antibody of isolated cardiomyocytes exposed to 1 μM isoproterenol. For cardiomyocytes isolated from α_{1C}-APEX2 and β_{2B}-APEX2 mice, the cardiomyocytes were exposed to isoproterenol after incubation with biotin-phenol. FIG. 14D shows a Western blot probed with phospho-phospholamban antibody for whole heart exposed to 1 μM isoproterenol for 5 minutes after infusion of biotin-phenol.

FIG. 15A is a dendrogram showing two-way hierarchical clustering of scaled TMT signal to noise (s/n) data for streptavidin-purified proteins from α_{1C}-APEX2 expressing cardiomyocytes under isoproterenol stimulation (iso) or with vehicle (no iso). Scaled relative TMT protein quantification data for 1951 proteins from biological quintuplicate α_{1C}-APEX2. FIG. 15B is a dendrogram showing two-way hierarchical clustering of scaled TMT signal to noise (s/n) data for streptavidin-purified proteins from α_{1C}-APEX2 expressing cardiomyocytes under isoproterenol stimulation (iso) or with vehicle (no iso). Scaled relative TMT protein quantification data for 1936 proteins from biological triplicate β_{2B}-APEX2 experiments. Heterogeneity between cardiomyocyte preparations from different mice is apparent. Clustering used Ward's minimum variance method. FIG. 15C is a dendrogram scaled with relative quantification data for 2,610 proteins from whole-organ α_{1C}-APEX without or with perfusion of isoproterenol (see FIG. 9D) used for two-way hierarchical clustering. Prominent heterogeneity in the proteins quantified between hearts is apparent. Position of Rad is indicated by a red line. In this experiment, the individual hearts were not paired. FIG. 16A shows the MS² spectrum and TMT quantification parameters of a Rad peptide changed upon isoproterenol treatment of murine hearts. The MS² spectrum used for identification of the peptide: IFGGIEDGPEAEAAGHTYDR mapping to Rad is displayed. y and b ion m/z identified in the spectrum and their deviation from theoretical m/z are displayed on the left. Precursor mass was measured as 778.71 Da with a charge of +3. Peptide modifications were +229.16 Da for TMT on the peptide N-terminus and lysine residues, +57.02 Da for cysteine alkylation and +15.99 for methionine oxidation. Ion injection times, isolation specificity, sum of signal to noise (SN) over all TMT channels, TMT raw intensities, adjusted intensities and final SN intensities used for relative quantification as well as SPS ion m/z are displayed.

FIG. 15D is a dendrogram showing two-way hierarchical clustering of scaled TMT signal to noise (s/n) data from non-transgenic (NTG) cardiomyocytes under isoproterenol stimulation (iso) or with vehicle (no iso). Scaled data of 4,622 quantified proteins from biological quadruplicate experiment are displayed. Pairing of samples is apparent. Clustering used Ward's minimum variance method. FIG. 16B is a table showing gene names of proteins with FDR-corrected P<0.05 for the three approaches: cardiomyocytes isolated from α_{1C}-APEX and β_{2B}-APEX mice, and α_{1C}-APEX hearts. Yellow-highlighted genes are in common amongst groups, but note that in some cases, fold-change is not consistent. FIG. 16C shows a Venn diagram of the data from FIG. 16B. Proteins: Rad=Rrad; Sorbin and SH3 domain-containing protein 2=Sorbs2; PKA catalytic subunit=Prkaca; Acss1=acyl-CoA synthetase short chain family member 1. Rad is the only protein consistently changed amongst the three approaches.

Rad is a potential PKA target. It is a member of the Rad- and Gem/Kir Ras-related (RGK) family of GTP-binding proteins, known for their capacity to inhibit all high-voltage-activated Ca²⁺ channels. Recently, a Rem2 variant was identified as a genetic modifier in long QT syndrome 2, and a Rad variant was linked to Brugada syndrome. Moreover, Rad-knockout mice display an increased maximum Ca²⁺ current, and the Ca²⁺ channels activate at lower voltages, mimicking the effects of β-adrenergic receptor stimulation. Other studies, however, led to expectations that Rad is not directly involved in adrenergic regulation of Ca_{V}1.2 since adenoviral-induced over-expression of Rad or Rem in cultured cardiomyocytes ablated Ca_{V}1.2 and attenuated adrenergic regulation. Our proximity labeling results elevated Rad as the leading candidate for the critical missing link that enables PKA regulation of Ca_{V}1.2.

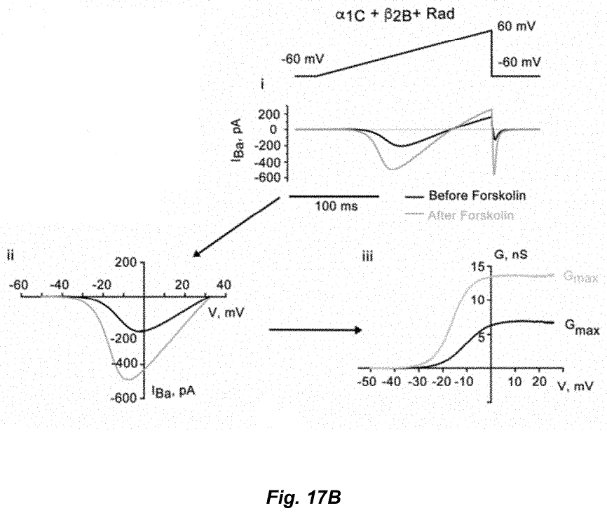

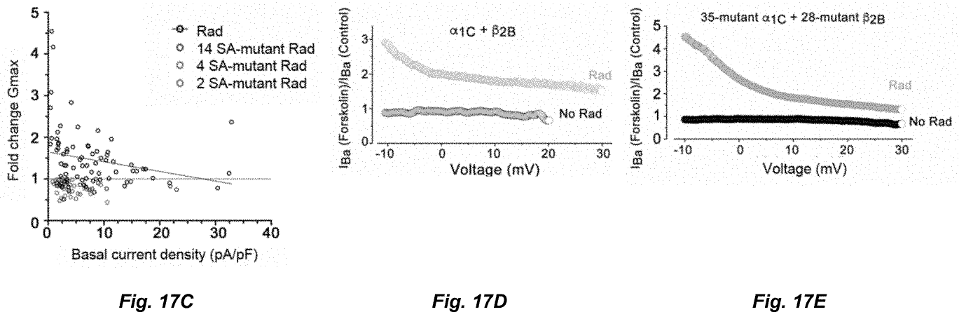

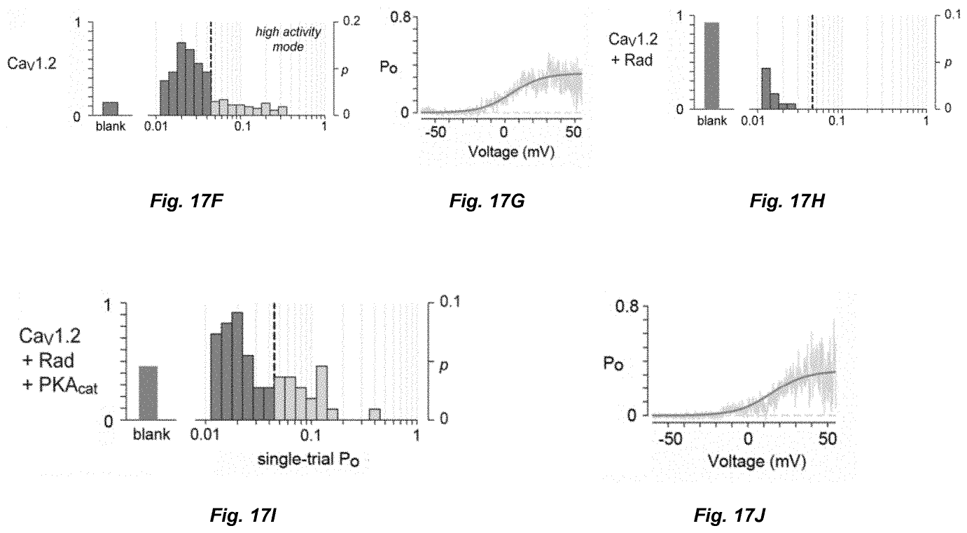

PKA regulation of Ca_{V}1.2 requires phosphorylation of Rad. The robust heterologous reconstitution of PKA regulation of L-type Ca²⁺ currents has been long pursued as a crucial step. Previous attempts have been unsuccessful or irreproducible. The inventors now find that Rad was the missing ingredient. To prove this experimentally in vivo, the inventors co-expressed Rad with α_{1C} and β_{2B} subunits in HEK293T cells, adding 3- to 6-fold less Rad than Ca_{V}1.2 subunits. Excess Rad could eliminate Ca²⁺ current. To preserve the normal intracellular milieu and signaling cascades, the inventors used perforated, whole-cell patch clamp to assess function. The inventors used Ba²⁺ as the charge carrier to minimize inactivation and depolarized the membrane potential with either a voltage step to 0 mV or a voltage ramp from −60 mV to +60 mV. Under these conditions, run-down of the Ba²⁺ current was minimal. In control HEK293T cells transfected with only α_{1C}+β_{2B}, superfusion of forskolin over 1-3 minutes had no impact on Ba²⁺ current (FIGS. 1A-1C and FIGS. 1J and 1K). In sharp contrast, in cells expressing α_{1C}+β_{2B}+Rad, applying forskolin increased the maximal conductance (Gₘₐₓ) by as much as 4.5-fold and by a mean of 1.5-fold, and shifted the V₅₀ for activation by −5 mV (FIGS. 1D-1F and FIGS. 1J and 1K; as well as FIGS. 17A and 17B). The forskolin-induced increase in current was inversely proportional to the basal current density (FIG. 17C).

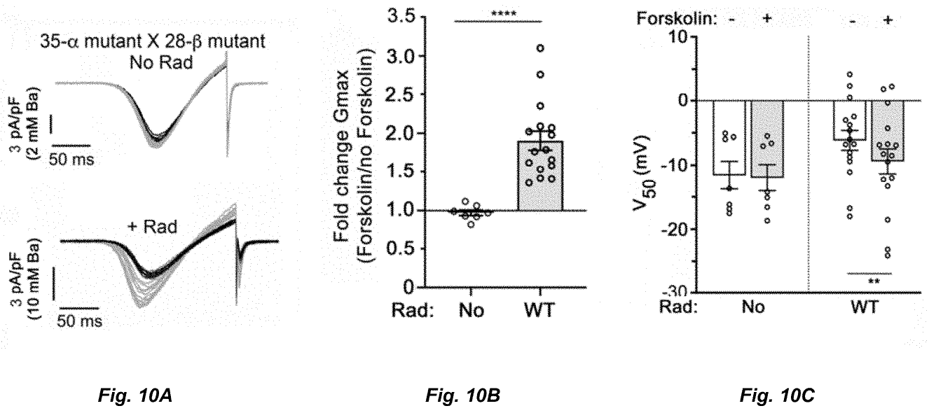

In HEK cells expressing 35-mutant α_{1C}+28-mutant β_{2B}+Rad, applying forskolin increased Gₘₐₓ by as much as 3.1-fold and by a mean of 1.9-fold, and shifted the V₅₀ for activation by −3.3 mV (FIGS. 10A-10C), consistent with the findings that acute β-adrenergic-stimulation of Ca_{V}1.2 does not require direct phosphorylation of α_{1C} or β₂ subunits in cardiomyocytes (FIGS. 7A-7J). For both WT and phospho-site mutant α_{1C} and β_{2B} subunits, the forskolin-induced enhancement of Ba²⁺ current in Rad-transfected cells was greatest at hyperpolarized potentials and fell as the test potential approached the reversal potential (FIGS. 17D and 17E), consistent with observations in cardiomyocytes.

FIGS. 1A, 1D and 1G schematically illustrate α_{1C}, β_{2B} and Rad at the membrane, respectively. FIGS. 1B, 1E, 1H and 1I are graphs showing Ba²⁺ current elicited by voltage ramp every 10 s from −60 mV to +60 mV over 200 ms. Black traces before and blue traces after forskolin. FIGS. 1C and 1F are diary plots of normalized Ba²⁺ current amplitude at 0 mV. FIG. 1J is a graph showing fold increase in maximum conductance (Gₘₐₓ) induced by forskolin. Mean±SEM. P<0.0001 by one-way ANOVA; ** P<0.01, **** P<0.0001 by Tukey's multiple comparison test. FIG. 1K is a graph showing the Boltzmann function parameter V_{mid}. *** P<0.001 by paired t-test. FIG. 10A shows exemplary traces of Ba²⁺ currents of 35-mutant α_{1C}+28-mutant β_{2B} in the absence and in the presence of Rad elicited by voltage ramp every 10 s from −60 mV to +60 mV over 200 ms. Black traces before and blue traces after forskolin. FIG. 10B is a graph showing a fold increase in maximum conductance (Gₘₐₓ) induced by forskolin for 35-mutant α_{1C}+28-mutant β_{2B} in the absence and presence of Rad. Mean±SEM. **** P<0.0001 by t-test. FIG. 10C is a graph showing the Boltzmann function parameter V_{mid}. ** P<0.01 by paired t-test. FIGS. 2A, 2B and 2C are graphs showing the stochastic records (top rows), where channel closures correspond to the zero-current portions of the trace (on horizontal gray lines) and openings to downward deflections to the open level (slanted gray curves). Averaging many records yields a mean current that can be divided into the open level (slanted gray curve) to furnish the Po versus voltage relation (sigmoidal trace at bottom), averaged over multiple patches. In all experiments, α_{1C} and β_{2B} were expressed in HEK cells with no Rad (FIG. 2A), WT Rad (FIG. 2B), or 4SA-mutant Rad (FIG. 2C), in the absence or presence of exogenous PKA catalytic subunit. Dashed line is Po for control of α_{1C}+β_{2B} without Rad. Control, n=10; Control+PKA, n=5; Rad, n=5; Rad+PKA, n=9; 4SA mutant Rad, n=6, 4SA mutant Rad+PKA, n=6.

The inventors used low noise single-channel recordings to determine the mechanism of PKA/Rad modulation of Ca_{V}1.2, and a slow voltage ramp was used to elicit stochastic channel opening that reflect near-steady-state open probability (Pₒ) at each voltage. In the exemplary records, channels closures correspond to zero-current portions (horizontal gray lines) and openings correspond to downward deflections to the open level (slanted gray lines). In the absence of Rad, sweeps with no openings or blank sweeps are quite rare (10%), while most sweeps exhibit either intermediate or high levels of openings, consistent with channels switching between low and high activity modes (FIG. 2A, and FIGS. 17F and 17G). In HEK cells transfected with α_{1C}+β_{2B}, co-expression of the catalytically-active PKA subunit had no effect on Pₒ (FIG. 2A). By contrast, when Rad is over-expressed the fraction of blank sweeps is markedly increased (90%), and the Pₒ is markedly reduced (FIG. 2B, and FIG. 17H). By comparison, if the PKA catalytic domain is also co-expressed with Rad, the fraction of blank sweeps is markedly reduced (˜0.45), there is a resurgence of the high activity mode, and the Pₒ increased by 10.6±2.9-fold compared to transfection without PKA (FIG. 2B, and FIGS. 171 and 17J). Altogether these results suggest that Rad potently silences Cav1.2 while phosphorylation of Rad relieves silenced channels allowing them to operate as though they are devoid of Rad.

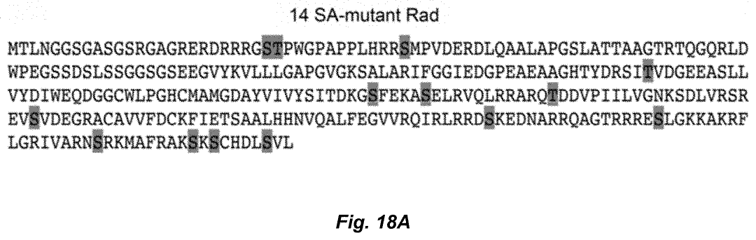

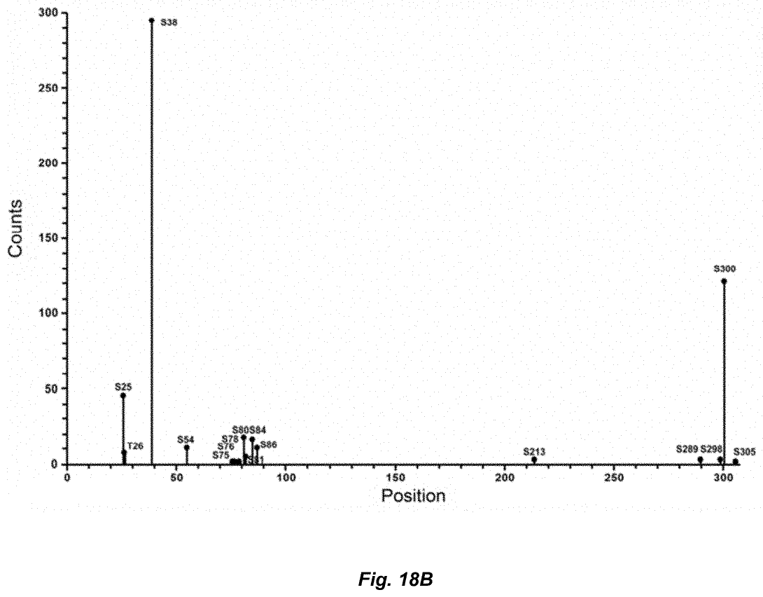

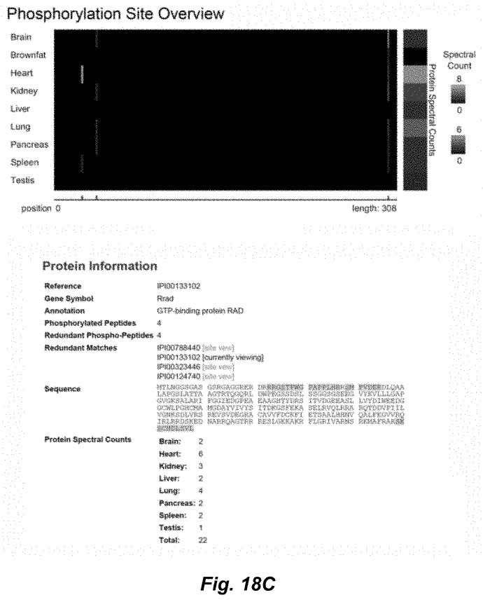

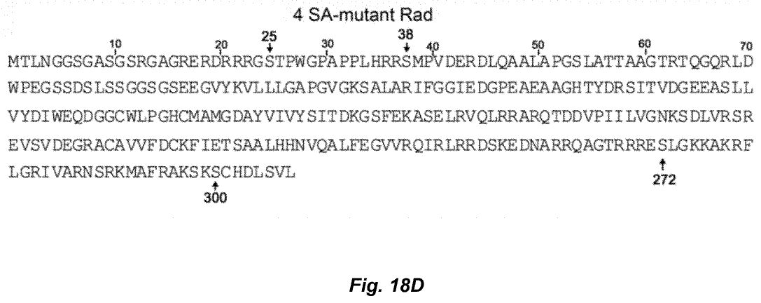

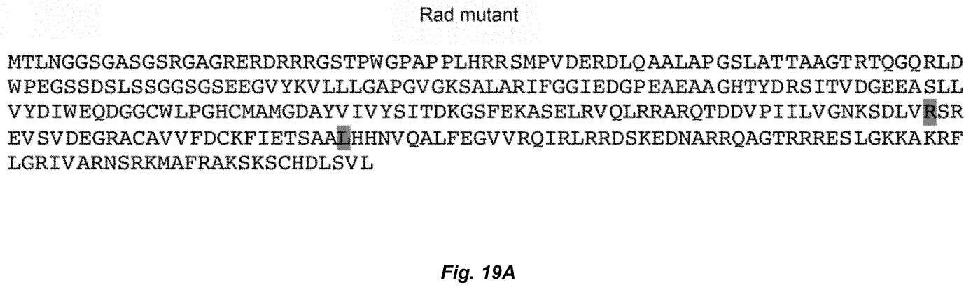

Having established that expression of Rad is required for PKA-mediated activation of Ca_{V}1.2 current in HEK cells, the inventors then asked whether Rad is the critical PKA-target. Using both manual sequence analyses and several web-based PKA phosphorylation prediction tools, the inventors identified 14 consensus PKA phosphorylation sites in Rad (residues labeled purple in FIG. 18A). The inventors mutated all 14 Ser/Thr residues to Ala. This mutant Rad still effectively inhibited Ca_{V}1.2 currents; however, the cAMP-PKA mediated upregulation of Ca_{V}1.2 current was lost (FIG. 1J).

In lysates from forskolin-stimulated HEK cells transfected with GFP-Rad, the inventors found, using mass spectrometry, that Ser²⁵, Ser³⁸ and Ser³⁰⁰ were phosphorylated (FIG. 18B). These residues were previously identified in the hearts of mice as phosphorylation targets (FIG. 18C). The inventors were unable to detect either non-phosphorylated or phosphorylated peptides containing Ser²⁷², notwithstanding prior biochemical studies identifying Ser²⁷² as a PKA target. Similar to the complete phosphorylation-site-deficient Rad, Ala substitutions of Rad at Ser²⁵, Ser³⁸, Ser²⁷² and Ser³⁰⁰ (4-SA mutant, FIG. 18D) prevented both the forskolin-induced increase in G. and hyperpolarizing shift in the I-V curve (FIGS. 1G, 1H, 1J and 1K). In contrast to transfection with WT Rad, co-transfection of the catalytically-active PKA subunit with 4-SA mutant Rad failed to substantially increase the open probability (PKA to no PKA: 1.15±0.56-fold; FIG. 2C).

FIG. 17A is an exemplary graph showing whole-cell Ca_{V}1.2 currents elicited from step depolarizations recorded from HEK cells expressing Rad. Pulses every 10 s from −50 mV to 0 mV before (black traces) and during forskolin (blue traces). FIG. 17B illustrates the methodology used for generating G/Voltage curves. For sub-graph (i), upper: 200 ms voltage ramp from −60 mV to +60 mV was applied every 10 s. Lower: Current traces—average of 3 traces before forskolin (black traces) and 3 traces after forskolin (blue traces). For sub-graph (ii), the data was processed to convert time scale to applied voltage and corrected for junction potential (10 mV). For sub-graph (iii), the data were processed as described below, with regard to the methods used to convert Ba²⁺ current to conductance. The fold change was calculated at Gₘₐₓ. FIG. 17C is a graph of forskolin-induced fold change in current stratified by current density before forskolin. FIGS. 17D and 17E are graphs showing the voltage-dependence of forskolin-induced augmentation of Ba²⁺ current, with exemplary graphs of ratio of peak current after forskolin to peak current before forskolin for cells transfected without and with Rad. In FIG. 17D, cells were transfected with WT α_{1C} and WT β_{2B}. In FIG. 17E, cells were transfected with 35-mutant α_{1C} and 28-mutant β_{2B}.

FIGS. 17F and 17G are graphs showing the distribution of sweep-by-sweep average Pₒ (single trial <P_{O}>) for Ca_{V}1.2 without Rad. FIG. 17H is a graph showing the distribution of sweep-by-sweep average P_{O} (single trial <Pₒ>) for Ca_{V}1.2 with Rad.

FIGS. 17I and 17J are graphs showing the distribution of sweep-by-sweep average P_{O} (single trial <P_{O}>) for Ca_{V}1.2 with Rad and PKA catalytic subunit co-expressed, respectively. FIG. 17F is a histogram of single trial <P_{O}> for Ca_{V}1.2 without Rad. In the absence of Rad, sweeps with no openings or blank sweeps are quite rare (10%), while most sweeps exhibit either intermediate or high levels of openings, consistent with channels switching between high and low activity modes. FIG. 17G is a graph showing the conditional Pₒ-voltage relationship obtained for sweeps exhibiting high activity. FIG. 17H is a histogram showing the fraction of blank sweeps is markedly increased (90%) with expression of Rad. FIG. 17I is a histogram showing the PKA catalytic domain also co-expressed with Rad, where the fraction of blank sweeps is markedly reduced (˜0.45) and there is a resurgence of the high activity mode. FIG. 17J is a graph showing the conditional Pₒ-voltage relationship computed only from sweeps exhibiting high activity. This relationship is similar to that observed in the absence of Rad (FIG. 17G).

FIG. 18A shows the genetic code for serine/Threonine residues mutated to alanine. FIG. 18B shows the mass spectrometry identification of phosphorylated residues on Rad enriched with an anti-GFP nanobody matrix from HEK cells expressing GFP-Rad and treated with forskolin. The number of spectral counts is plotted against the position of the phosphorylated amino acids in Rad. 534 phosphopeptides were detected. FIG. 18C shows the database entry of phosphorylation sites identified previously in Rad. The highest level of Rad phosphorylation was detected in the heart. Peptides with phosphorylated Ser residues (bold, red) on positions 25, 38 and 300 (mapped to Rad expression constructs used in this study) were detected. FIG. 18D shows the genetic code for serine residues mutated to alanine in 4-SA (mutant indicated by arrows).

A C-terminal 32 amino acids, polybasic region of Rad is involved in plasma membrane targeting via binding to anionic phospholipids such as PIP2. Deletion of these 32 residues prevents Rad-mediated inhibition of Ca²⁺ channel function. Deletion of the C-terminal domain of Rem, a homologous RGK GTPase, also greatly reduced binding of Rem to the Ca_{V} β subunit. The inventors reasoned that phosphorylation of the two serine residues, Ser²⁷² and Ser³⁰⁰, within the C-terminal polybasic membrane region can tune the Rad-mediated inhibition of Ca²⁺ channels, potentially by changing the electrostatic interactions with anionic phospholipids. Consistent with this hypothesis, the inventors found that alanine substitutions at Ser²⁷² and Ser³⁰⁰ (2-SA mutant) in the C-terminus prevented both the forskolin-induced increase in current amplitude and the hyperpolarizing shift in the I-V curve (FIGS. 1I-1K; and FIG. 17C).

FIG. 18C illustrates phosphorylation sites identified previously in Rad. The highest level of Rad phosphorylation was detected in the heart. Peptides with phosphorylated Ser residues (bold, red) on positions 25, 38 and 300 (mapped to Rad expression constructs used in this study) were detected.

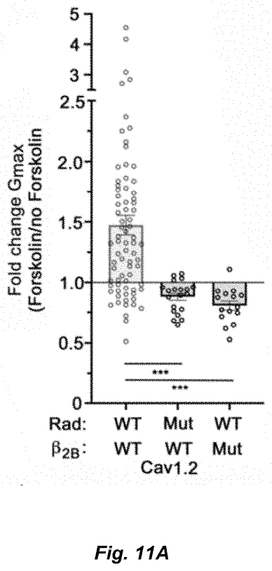

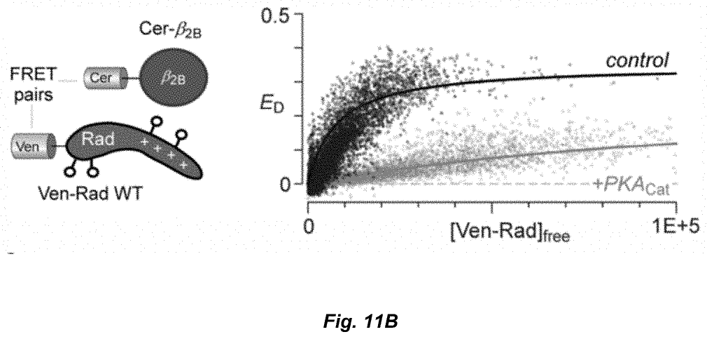

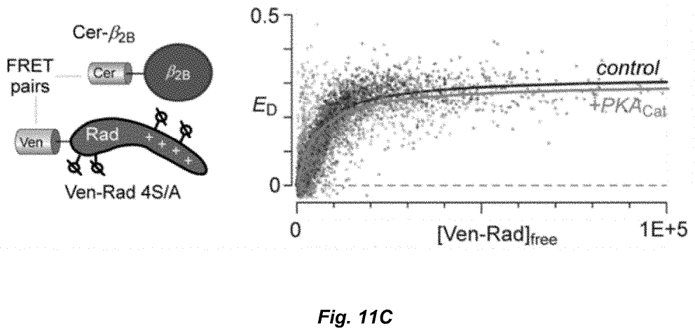

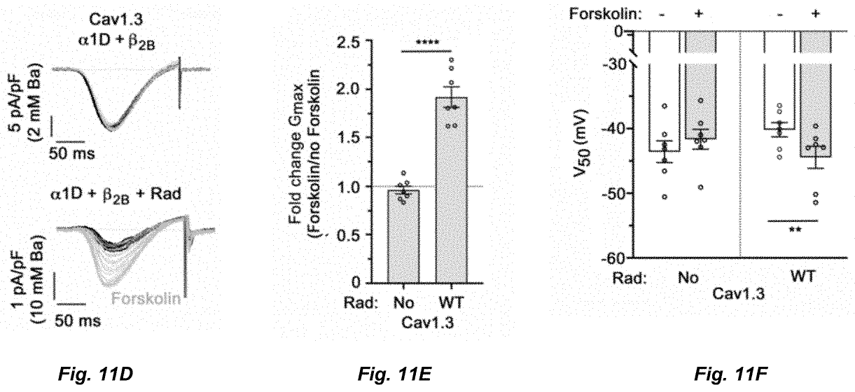

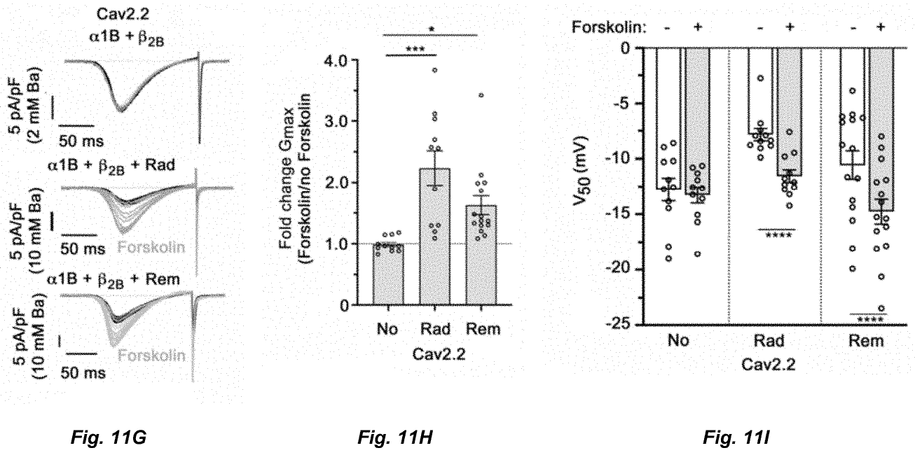

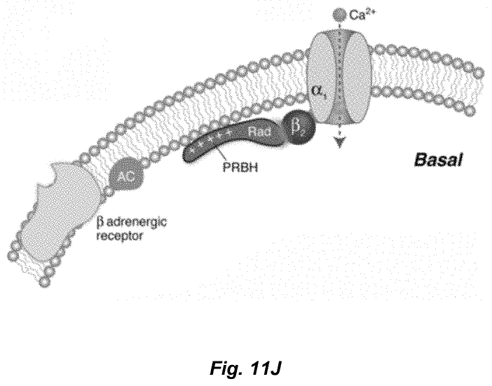

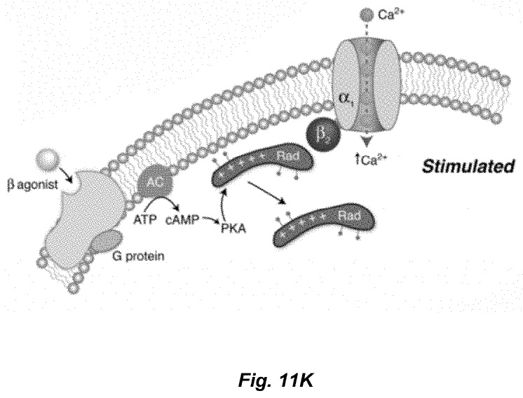

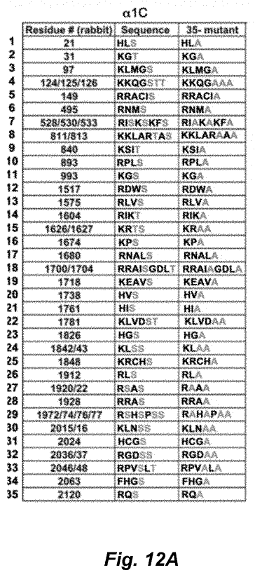

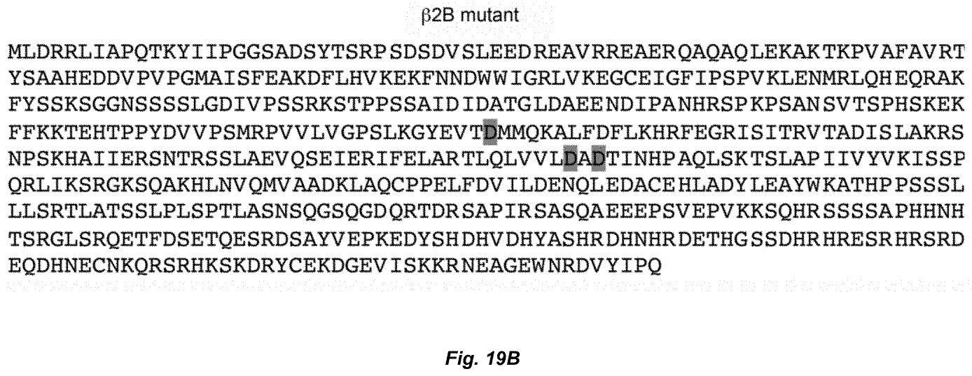

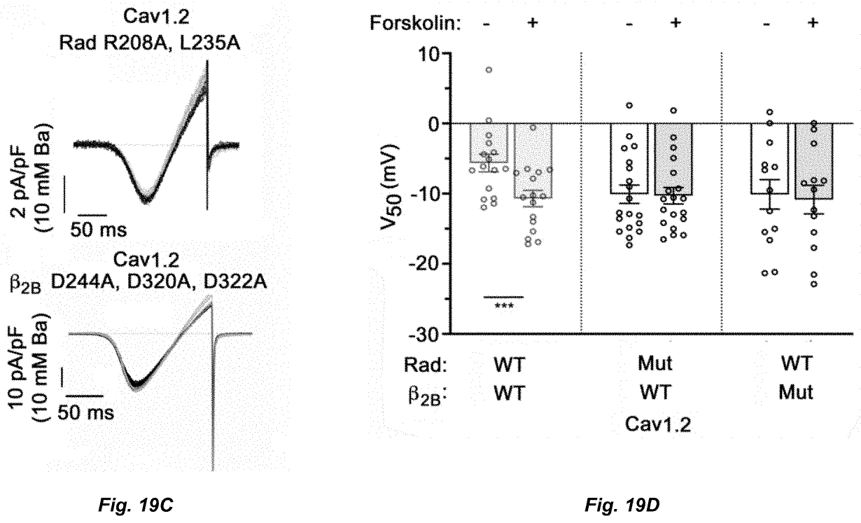

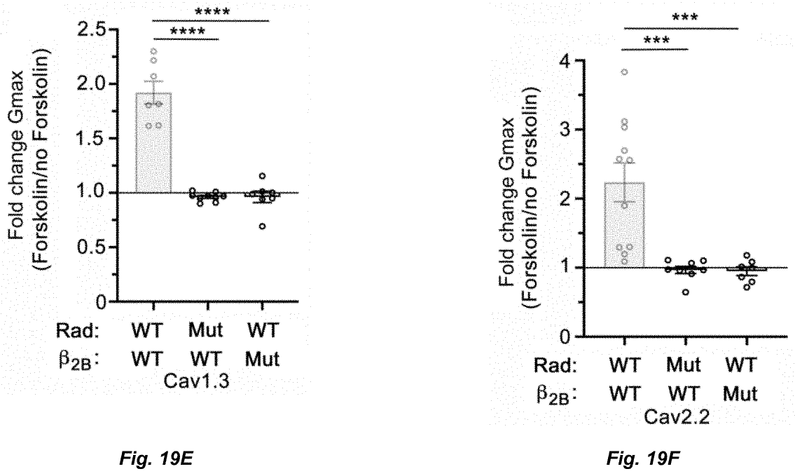

FIG. 19A shows the genetic code for alanine substitutions of Rad at residues R208 and L235 (yellow), and FIG. 19B shows the genetic code for alanine substitutions at residues D244, D320 and D322 (yellow) of β_{2B}, which were created to attenuate Rad binding to β. FIG. 19C is a graph showing the Ba²⁺ current of Ca_{V}1.2 channels elicited by voltage ramp every 10 s from −60 mV to +60 mV over 200 ms. Black traces before and blue traces after forskolin. FIG. 19D is a graph showing the Boltzmann function parameter V₅₀. ** p<0.001 by paired t-test. The data for Rad is same as FIG. 1K. FIGS. 19E and 19F are graphs showing the fold changes (forskolin vs. no forskolin, respectively) in Gₘₐₓ. Mean±SEM. P<0.0001 by one-way ANOVA; *** P<0.001, **** P<0.0001 by Dunnett multiple comparisons test. Data for WT Rad and WT β_{2B} is same as that of FIG. 11E and FIG. 11H, respectively.

Rad binding to 0 is required for PKA regulation of Ca_{V}1.2 channels. Rad can inhibit Ca_{V}1.2 via β-dependent and β-independent (α_{1C}-dependent) mechanisms. Alanine substitutions of Rad at residues R208 and L235 (FIG. 19A), and alanine substitutions at residues D244, D320 and D322 of β_{2B} (FIG. 19B) attenuate Rad binding to β. Attenuating binding of Rad to β, via these mutations, prevented the forskolin-induced increase in Gₘₐₓ (FIG. 11A; and FIG. 19C) and the hyperpolarizing shift in the I-V curve (FIG. 19D). These findings highlight the essential requirement of Rad-β subunit interaction for the cAMP-PKA regulation of Ca_{V}1.2.

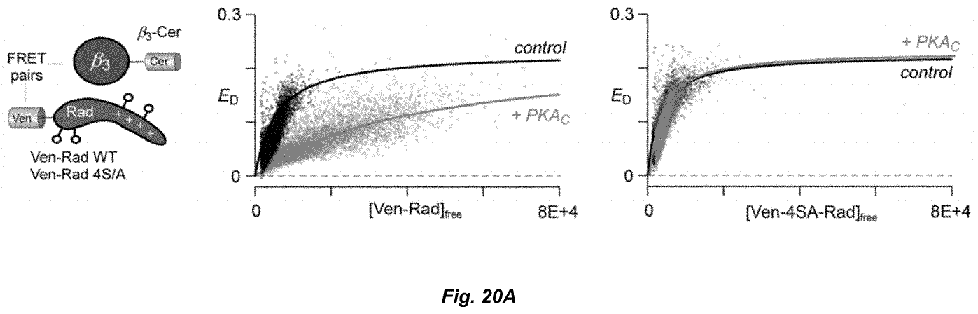

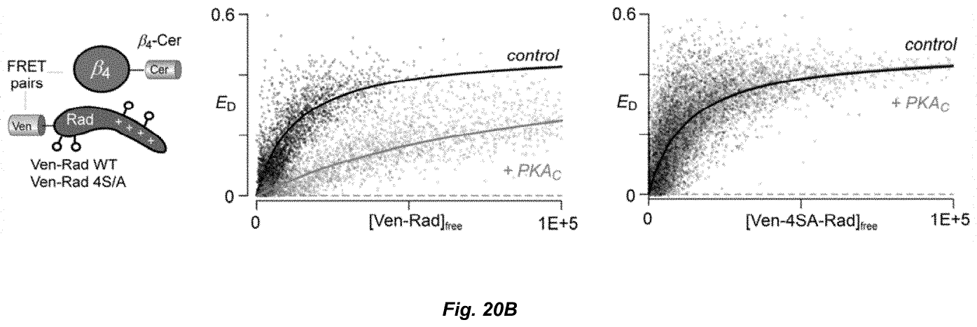

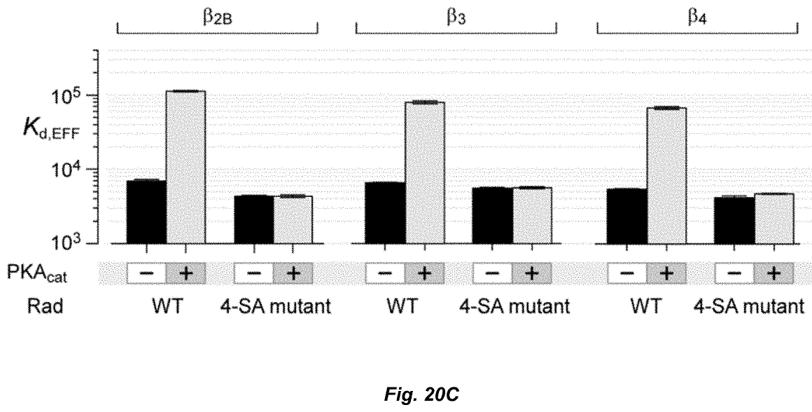

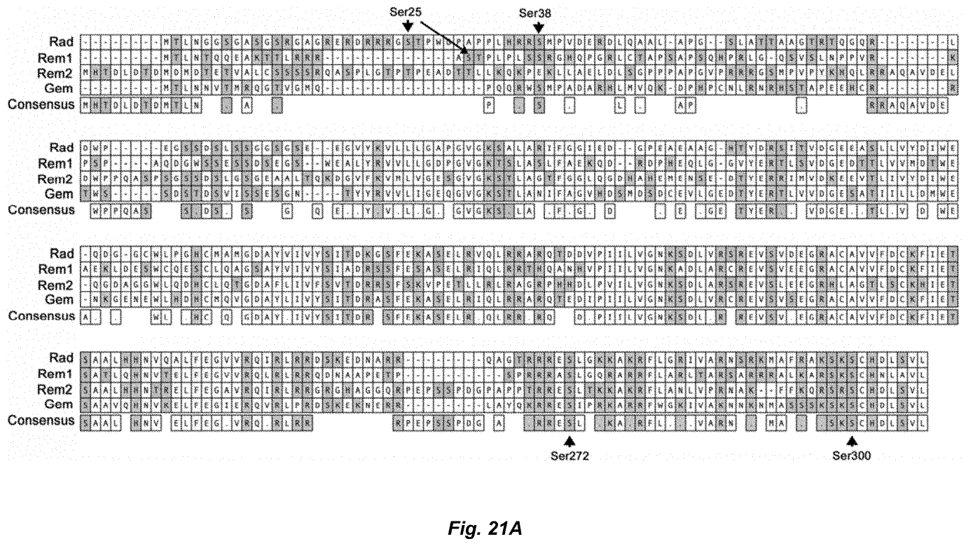

The inventors utilized flow-cytometric Förster resonance energy transfer (FRET) 2-hybrid assay to probe potential PKA-mediated changes in the binding of β_{2B} subunit and WT Rad (FIGS. 11B and 11C, and FIG. 20C). At baseline, robust binding is detected between cerulean-tagged β_{2B} subunit and venus-tagged WT Rad, consistent with previous studies (K_{d,EFF}=6925±363, FIG. 11B; and FIG. 20C). Co-expression of PKA catalytic subunit, however, markedly weakened this interaction (K_{d,EFF}=112558±2442). In contrast, co-expression of the PKA catalytic subunit in cells expressing fluorophore-tagged β_{2B} and 4-SA mutant Rad had no effect on FRET binding (K_{d,EFF}=4349±138 versus K_{d,EFF}=4346±197 with and without PKA catalytic domain respectively; FIG. 11C, and FIG. 20C). These results suggest that phosphorylation of Rad is required for dissociation of the Rad-β_{2B} interaction. In like manner, PKA phosphorylation of Rad also reduced FRET binding to both β₃ and β₄ (FIGS. 20A-20C). Thus, the phosphorylation-dependent dissociation of Rad and β_{2B} is conserved amongst other β subunits.

FIGS. 20A and 20B illustrate FRET 2-hybrid binding isotherms determined for Cerulean (Cer)-tagged β₃ and β₄, and N-terminal Venus (Ven)-tagged WT or 4-SA mutant Rad. FRET efficiency (E_{D}) is plotted against the free concentration Ven-WT or Ven-4SA-mutant Rad. The solid line fits a 1:1 binding isotherm. Co-expression of the PKA catalytic subunit weakened binding in WT Rad-expressing cells, but not 4-SA mutant Rad-expressing cells. FIG. 20C is a bar graph summarizing mean K_{d,EFF} for β_{2B}, β₃ and β₄, and WT and 4-SA mutant Rad, expressed without and with catalytic PKA subunit. Error bars on the K_{d,EFF} is a 95%-CI for the pooled non-linear fits based on the Jacobians computed.

The FRET-based method for assessing Rad/β binding demonstrates that Rad binds to β₃ and β₄, and that this can be reversed by PKA phosphorylation of Rad dependent. Cerulean (Cer)-tagged β₃ (FIG. 20A) or β₄ (FIG. 20B) is co-expressed in HEK cells with either N-terminal Venus (Ven)-tagged WT or 4-SA mutant Rad (i.e., Rad that cannot be phosphorylated). FRET efficiency (E_{D}) is plotted against the free concentration Ven-WT or Ven-4SA-mutant Rad. The solid line fits a 1:1 binding isotherm. Co-expression of the PKA catalytic subunit weakened binding in WT Rad-expressing cells, but not 4-SA mutant Rad-expressing cells. FIG. 20C is a bar graph summarizing mean K_{d,EFF} for β_{2B}, β₃ and β₄, and WT and 4-SA mutant Rad, expressed without and with catalytic PKA subunit. The error bars on the K_{d,EFF} are a 95%-CI for the pooled non-linear fits based on the Jacobians computed.

Rad and Rem confer PKA regulation to Ca_{V}1.3 and Ca_{V}2.2 channels. In adrenal chromaffin cells and the sinus node cells of the heart, L-type Ca_{V}1.3 channels are robustly stimulated by PKA. In control HEK293T cells transfected with only Ca_{V}1.3 α_{1D}+β2B, superfusion of forskolin over 1-3 minutes had no impact on Ba²⁺ current (FIGS. 11D-11F). In sharp contrast, in cells expressing α_{1D}+β_{2B}+Rad, applying forskolin increased G. by as much as 2.3-fold and by a mean of 1.9-fold (FIGS. 11D and 11E), and shifted the V₅₀ for activation by −4.3 mV (FIG. 11F). Attenuating binding of Rad to 13 prevented the forskolin-induced modulation of Ca_{V}1.3 current (FIG. 19E). Thus, like Ca_{V}1.2, Rad is the missing ingredient.

FIG. 11A is a plot showing the fold change (forskolin vs. no forskolin) in Gₘₐₓ. Mean±SEM. P<0.0001 by one-way ANOVA; *** P<0.001 by Dunnett multiple comparisons test. Data for Rad is the same as that for FIG. 1J. FIGS. 11B and 11C illustrate FRET 2-hybrid binding isotherms determined for Cerulean (Cer)-tagged β_{2B} and N-terminal Venus (Ven)-tagged WT or 4-SA mutant Rad. FRET efficiency (E_{D}) is plotted against the free concentration Ven-WT (FIG. 11B) or Ven-4SA-mutant Rad (FIG. 11C). The solid line fits a 1:1 binding isotherm. Co-expression of the PKA catalytic subunit weakened binding in WT Rad-expressing cells (FIG. 11B), but not 4-SA mutant Rad-expressing cells (FIG. 11C). FIG. 11D is a graph showing Ba²⁺ current of Ca_{V}1.3 channels without or with expression of Rad elicited by voltage ramp every 10 s from −60 mV to +60 mV over 200 ms. Black traces before and blue traces after forskolin. FIG. 11E is a graph showing fold changes (forskolin vs. no forskolin) in Gₘₐₓ. Mean±SEM. **** P<0.0001 by unpaired t-test. FIG. 11F is a graph showing the Boltzmann function parameter V₅₀. ** P<0.01 by paired t-test. FIG. 11G is a graph showing Ba²⁺ current of Ca_{V}2.2 channels without or with expression of Rad or Rem elicited by voltage ramp every 10 s from −60 mV to +60 mV over 200 ms. Black traces show before forskolin and blue traces show after forskolin. FIG. 11H is a graph showing fold change (forskolin vs. no forskolin) in Gₘₐₓ. Mean±SEM. P<0.001 by one-way ANOVA; **** P<0.0001, *** P<0.001, * P<0.05 by Dunnett multiple comparisons test. FIG. 11I is a graph showing the Boltzmann function parameter V₅₀. **** P<0.001 by paired t-test. FIGS. 11J and 11K schematically illustrate β-adrenergic regulation of Ca²⁺ channels. β-agonist-induced activation of PKA leads to PKA phosphorylation of Rad causing dissociation of Rad from the Ca_{V}1.2 complex, and subsequently increased Ca²⁺ influx. AC: adenylyl cyclase, PRBH: phospho-regulatory basic hydrophobic motif.

FIG. 11A demonstrates phosphorylation dependent regulation of Cav1.2 by Rad and β₂. In the presence of wildtype Rad and β₂, the addition of forskolin (i.e., an activator of PKA) induces a fold increase in Cav1.2 current. A similar observation is made with Cav1.3 (FIGS. 11D, 11E and 11F) and Cav2.2 (FIGS. 11G, 11H and 11I). A similar observation is also made with Rem, a member of the RGK GTPase family of proteins (FIGS. 11G and 11H), demonstrating that this mechanism for regulating calcium channels is likely an evolutionary conserved mechanism.

β₂ binding to Rad was demonstrated using a novel FRET-based assay. Cerulean (Cer)-tagged β_{2B} is co-expressed in HEK cells with either N-terminal Venus (Ven)-tagged WT (FIG. 11B) or 4-SA mutant Rad (i.e., Rad that cannot be phosphorylated) (FIG. 11C). FRET 2-hybrid binding isotherms were determined for FRET efficiency (E_{D}), which is plotted against the free concentration Ven-WT (FIG. 11B) or Ven-4SA-mutant Rad (FIG. 11C). The solid line fits a 1:1 binding isotherm. Co-expression of the PKA catalytic subunit weakened binding in WT Rad-expressing cells (FIG. 11B), but not 4-SA mutant Rad-expressing cells (FIG. 11C). FIG. 11J is a schematic of β-adrenergic regulation of Ca²⁺ channels. β-agonist-induced activation of PKA leads to PKA phosphorylation of Rad, causing dissociation of Rad from the Ca_{V}1.2 complex, and subsequently increased Ca²⁺ influx.

N-type Ca²⁺ channels (Ca_{V}2.2) are widely expressed, play an essential role in neurotransmitter release, and are targets for the development of drugs to relieve chronic pain. The inventors expressed the Ca_{V}2.2 α_{1B} subunit with β_{2B} and Rad in HEK cells. As with Ca_{V}1.2 and Ca_{V}1.3, forskolin increased Gₘₐₓ through Ca_{V}2.2 when co-expressed with Rad by a mean of 2.2-fold and shifted the V₅₀ for activation by −3.7 mV (FIGS. 11G-11I). Attenuating binding of Rad to 13 prevented the forskolin-induced modulation of Ca_{V}2.2 current (FIG. 19F).

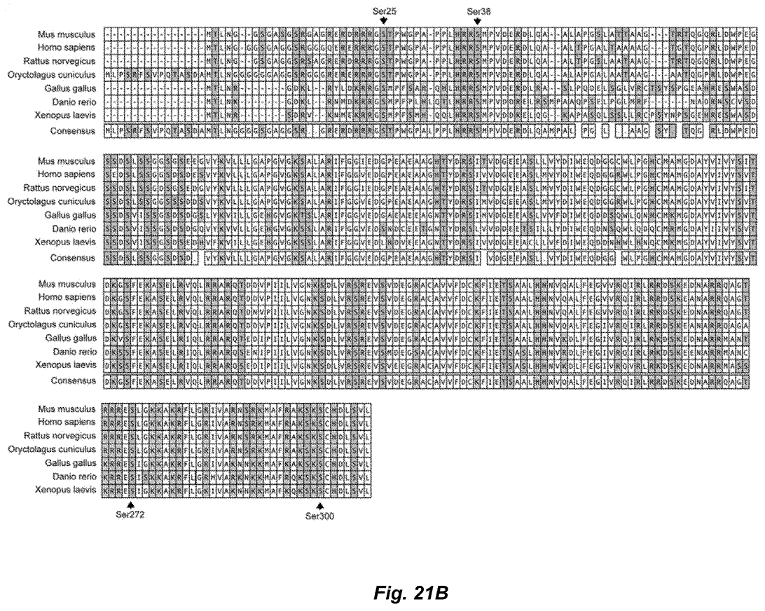

Multiple-alignment analysis of mouse Rad and other species shows conservation of the four phosphorylation sites, suggesting that this regulatory mechanism is conserved (FIG. 21B). Furthermore, analysis of Rad and other members of the RGK GTPase family indicate a high degree of similarity of phosphorylation sites on the C-terminus of the protein (FIG. 21A). The inventors expressed in HEK cells the Ca_{V}2.2 α_{1B} subunit with β_{2B} and Rem. Forskolin increased Gₘₐₓ through Ca_{V}2.2 when co-expressed with Rem by 1.6-fold and shifted the V₅₀ for activation by −4.2 mV (FIGS. 11G-11I). Thus, PKA-modulation of Ca_{V} channels is not idiosyncratic as currently believed; rather, it is emerging to be a universal mechanism transferable to all Ca_{V} channels that bind 13 subunits.

FIG. 21A shows the ClustalW alignment of murine Rad, Rem1, Rem2, and Gem sequences, with phosphorylation sites for mouse Rad Ser²⁵, Ser³⁸, Ser²⁷² and Ser³⁰⁰ being indicated with arrows. The C-terminal phosphorylation sites are conserved. The equivalent of Ser²⁵ phosphorylation site is conserved in Rem1. The equivalent of Ser³⁸ phosphorylation site is probably conserved in Gem. Blue highlights are basic amino acids (Arg, Lys and His), and red highlights are Ser and Thr. FIG. 21B shows the ClustalW alignment of Rad sequences, with conserved phosphorylation sites for mouse Ser²⁵, Ser³⁸, Ser²⁷² and Ser³⁰⁰ being shown. Blue highlights are basic amino acids (Arg, Lys and His), and red highlights are Ser and Thr.

PKA-induced stimulation of the Ca_{V}1.2 Ca²⁺ current is the prototypic form of Ca²⁺ channel modulation. Yet decades of research had failed to identify the molecular mechanisms responsible. It was supposed that the core α_{1C} and β_{2B} subunits contain the PKA target sites required for acute β-adrenergic agonist-induced stimulation of Ca_{V}1.2, but as the inventors demonstrate here they do not (FIGS. 7A-7J). Successful reconstitution of regulation required an unknown missing component, which the inventors show here is the small G-protein Rad. This protein is the key regulator and PKA-target of sympathetic nervous system regulation of Ca²⁺ influx in the heart. Using transgenic expression of Ca_{V}1.2-APEX fusion proteins in mouse hearts combined with isobaric peptide labeling and relative quantitation by mass spectrometry, the inventors show an isoproterenol-dependent dynamically changing protein interaction network in the Ca_{V}1.2 microdomain marked by a reduction in Rad. Subsequently, the inventors found that when expressed with the core α_{1C} and β_{2B} subunits, Rad imparted cAMP-PKA regulation on Ca_{V}1.2, which required both the phosphorylation by PKA on the C-terminus of Rad (FIGS. 1A-2C) and the interaction of Rad with the 13 subunit (FIGS. 11A-11K). The required interaction with the 13 subunit is consistent with our recent findings showing that disrupting the α_{1c}-β interaction prevented PKA regulation of Ca_{V}1.2.

Short stretches of basic and hydrophobic (BH) amino acids are known to interact with the membrane and phosphorylation of residues within these alter the electrostatic character, thereby reducing membrane affinity. The phospho-regulated BH (PRBH) motif of Rad is an essential switch that couples the evolutionary-conserved fight or flight pathway to enhanced Ca²⁺ current (FIGS. 11J and 11K). Multiple-alignment analyses of mouse Rad and other species, and Rad and other RGK GTPases show conservation of the four phosphorylation sites, suggesting that this regulatory mechanism is conserved. The mechanism of regulation is modular and transferable as Ca_{V}1.3 channels and neuronal Ca_{V}2.2 channels are also imparted with forskolin-PKA mediated upregulation by Rad and Rem. The activation of Ca²⁺ channels via release of inhibition by PKA phosphorylation is reminiscent of PKA phosphorylation of phospholamban, an inhibitor of SERCA.

Whereas phosphorylation of Ser¹⁹²⁸ is definitively not required for β-adrenergic regulation of Ca_{V}1.2 in the heart, alanine substitution of Ser¹⁹²⁸ prevents β-adrenergic stimulation of Ca²⁺ channels in hippocampal neurons and hyperglycemia-induced stimulation of Ca²⁺ currents in arterial smooth muscle cells. Perhaps the components of the Ca_{V}1.2 channel macromolecular complex differ across tissues. Phosphorylation on the α_{1C} subunit may also affect the trafficking and clustering in neurons and cardiomyocytes.

Our results indicate that proximity labeling using APEX2 is feasible in heart and combined with multiplexed TMT proteomics can identify a dynamically evolving network of interactions induced by β-adrenergic stimulation. Our findings establish the utility of proximity labeling in animals and provide an important foundation for future studies that will investigate how diseases, such as heart failure, change the proteomic subdomain of the excitation-contraction coupling machinery.

These findings also identify potential targets and interaction sites for the therapeutic modulation of β-adrenergic regulation of Ca²⁺ currents in the heart and other tissues. For instance, disrupting the interaction between Rad and β subunit can be inotropic by increasing Ca²⁺ entry in the heart. Conversely, interfering with the α_{1C}-β interaction, blocking PKA phosphorylation of Rad or potentially enhancing the interaction of Rad with the plasma membrane could attenuate more specifically than β-blockers the sympathetic nervous system activation of cardiac Ca²⁺ entry and inotropy.

In the above, with regard to clone construction and cell culture, all mouse N-terminal-GFP-tagged mouse Rad (accession #XM_006531206) constructs in a pEGFP-C1 vector were generated by gene-synthesis (Gene Universal). The human Ca_{V}2.2 (pSAD442-1) was a gift from Diane Lipscombe (Addgene plasmid #62574). HEK293T cells were cultured in DMEM with 10% FBS, 1% Pen/Strep, and transfected with 3 μg rabbit α_{1C} (accession #X15539) or α_{1B} (for Ca_{V}2.2 experiments), 3 μg human β_{2B} (NM-201590.3) and 0.5 μg N-terminal-GFP-tagged mouse Rad using Lipofectamine 2000 (Thermo Fisher Scientific). The media was changed 4-6 hours after transfection. The cells were split onto coverslips coated with attachment factor protein (Gibco). Electrophysiological recordings were carried out at room temperature 24-48 hours after transfection.

For, animal generation, the Institutional Animal Care and Use Committee at Columbia University approved all animal experiments. The transgenic constructs were generated by fusing rabbit α_{1C} cDNA or human β_{2B} cDNA to the modified murine α-myosin heavy chain (MHC) tetracycline-inducible promoter vector (gift of Jeffrey Robbins and Jeffrey Molkentin, University of Cincinnati, Cincinnati, Ohio). A 3× FLAG-epitope was ligated in-frame to the N-terminus of α_{1C}. The α_{1C} subunit was engineered to be dihydropyridine (DHP)-insensitive with the substitutions T1066Y and Q1070M. GFP was ligated to the N-terminus of β_{2B}. The V5 epitope and APEX2 cDNA, created by gene synthesis, were conjugated to the N-terminus of DHP-resistant α_{1C} and β_{2B}.

The 35-α mutant and the 28-β mutant cDNA were generated by site-directed mutagenesis. The optimal PKA phosphorylation motif is a tetrapeptide with Arg at the 2nd and 3rd positions (termed −2 and −3) prior to the phosphorylated Ser or Thr, and a large hydrophobic residue immediately thereafter (R-R-X-S/T-Φ). The requirement for a positive charge is highest for residues at −2 and −3, but can be found for residues as far as position −6 in PKA target sites. Sites with Arg in positions between −4 and −1 are strongly preferred and to a lesser extent this is found for for His or Lys. The inventors identified all potential intracellular PKA phosphorylation sites (FIGS. 12A and 12D) in rabbit α_{1C} and human β_{2B} using both manual sequence analysis and several web-based PKA phosphorylation prediction tools, including pKaPS, DISPHOS, GPS, NETPHOS and SCANSITE. Each phosphorylation site was mutated to Ala. The inventors also mutated additional Ser and Thr within several amino acid residues C-terminal to the Arg or Lys, in order to ensure that we fully captured each phospho-regulatory site. 51 residues in rabbit α_{1C} were replaced with Ala at 35 potential phospho-regulatory domains in the 35-mutant α_{1C} construct, and 37 residues were replaced with Ala at 28 putative phospho-regulatory domains of β_{2B}. The inventors excluded those sites predicted to be extracellular or within the plasma membrane.

Transgenic mice with non-targeted insertion of these tetracycline-regulated cDNA were bred with cardiac-specific (α-MHC), doxycycline-regulated, codon-optimized reverse transcriptional trans-activator (rtTA) mice (obtained via the Mutant Mouse Resource and Research Center) to generate double-transgenic mice. For the α_{1C}-APEX2 and β_{2B}-APEX2 mice, transgene expression did not require doxycycline due to a low basal binding of rtTA protein to the Tet operator sequences (so-called “leak”). These expression levels result in Ca²⁺ current levels similar to naïve conditions in heart. The results presented were consistent across all founder lines and gender, and therefore were pooled.

With regard to the isolation of adult cardiac myocytes, mice ventricular myocytes were isolated by enzymatic digestion using a Langendorff perfusion apparatus. Cardiomyocytes were isolated from 8-12 week-old non-transgenic and transgenic mice.

For fractional shortening, freshly isolated myocytes were perfused with a Tyrode's solution containing 1.8 mM CaCl₂. Myocytes were field stimulated at 1-Hz. Nisoldipine (300 nM) dissolved in Tyrode's solution was then superfused. Fractional shortening of sarcomere length was measured using the SarcLen module of Ionoptix. With regard to whole cell patch clamp electrophysiology, isolated cardiomyocytes or HEK cells on glass 8×8 mm coverslips were placed in Bioptechs Delta T Dishes filled with solution containing (in mM): 112 NaCl, 5.4 KCl, 1.7 NaH₂PO4. 1.6 MgCl₂, 20.4 HEPES (pH 7.2), 30 Taurine, 2 DL-Carnitine, 10.3 Creatine, 5.4 Glucose. The petri dishes were mounted on the stage of an inverted microscope and served as a perfusion chamber. After establishing a seal and achieving whole-cell configuration, external solutions were changed by fast local perfusion method.

For cardiomyocytes, pipette resistance was between 1-3 ma Membrane currents were measured by conventional (ruptured) whole-cell patch-clamp method using a MultiClamp 700B or Axopath200B amplifier and pCLAMP 10.7 software (Molecular Devices). Capacitance transients and series resistance were compensated. Voltage was corrected for liquid junction potential (−10 mV) during analysis. Leak currents were subtracted by a P/4 protocol. The parameters of voltage-dependent activation were obtained using a modified Boltzmann distribution: I(V)=Gₘₐₓ*(V−Eᵣₑᵥ)/[1+exp(V−V₅₀)/V_{c})], where I(V) is peak current, Gₘₐₓ is maximal conductance, Eᵣₑᵥ is reversal potential, V₅₀ is the midpoint, and V_{c} is the slope factor.

The pipette solution contained was (in mM): 40 CsCI, 80 Cesium Gluconate, 10 BAPTA (1,2-bis(o-aminophenoxy)ethane-N,N,N′,N′-tetraacetic acid), 1 MgCl₂, 4 Mg-ATP, 2 CaCl₂, and 10 HEPES (4-(2-hydroxyethyl)-1-piperazineethanesulfonic acid), adjusted to pH 7.2 with CsOH. After the isolated cardiomyocytes were dialyzed and adequately buffered with 10 mM BAPTA in the internal solution, cells were superfused with (in mM): 140 TEA-Cl (Tetraethylammonium chloride), 1.8 CaCl₂, 1 MgCl₂, 10 glucose, and 10 HEPES, adjusted to pH 7.4 with CsOH. To measure peak currents, the cell membrane potential was held at −50 mV and stepped to +0 mV for 350 ms every 10 s. To evaluate the current-voltage (I-V) relationship in cardiomyocytes, the same protocol was repeated with steps between −60 mV to +60 mV in 10 mV increments. Nisoldipine (Santa Cruz) was stored protected from light at −20° C. as 3 mM stock in ethanol. The final dilution of nisoldipine to 300 nM was in the extracellular recording solution immediately prior to the experiment. Isoproterenol (Sigma 15627) and forskolin (Santa Cruz) were prepared daily and diluted in extracellular solution.

For HEK293T cell experiments involving the forskolin-induced stimulation of Ca_{V}1.2 and Ca_{V}2.2 currents, perforated whole-cell patch clamp technique was implemented to minimize current run-down and preserve the intracellular milieu. Amphotericin B (Sigma A9528) was initially dissolved in DMSO (20 mg/ml) and used in the pipette solution at a final concentration of 200 μg/ml. The tip of the pipette was filled with amphotericin-free solution (in mM): 80 Cesium Gluconate, 40 CsCI, 10 HEPES, 10 BAPTA, 1 MgCl₂, 1 Mg-ATP, pH adjusted to 7.2 with CsOH. The pipette was backfilled with (in mM): 125 CsCI, 10 HEPES, 4 CaCl₂, 1 MgCl₂, pH-7.2 with CsOH; containing amphotericin, 200 μg/ml. CaCl₂ (4 mM) was added to the intracellular patch electrode solution to enable the detection of conversion from perforated to ruptured configuration. The external solution contained (in mM): 130 tetraethylammonium methanesulfonate, 10 HEPES, 1 MgCl₂, 10 (with Rad expression) or 2 (without Rad expression) BaCI₂, 5 Glucose, pH adjusted to pH 7.4 with CsOH. For experiments with HEK293T cells, in addition to step protocols, we used a ramp protocol with a 200 ms voltage ramp from −60 mV (or −70 mV for Ca_{V}1.3) to +60 mV applied every 10 s- to monitor the near-steady-state I-V relationship. All experiments were performed at room temperature, 22±1° C. Cells were selected based on co-transfection of a vector containing GFP in the absence of Rad, and GFP-conjugated Rad for the experiments with Rad transfected. For both cardiomyocytes and HEK293 cells, cells without a stable baseline (potentially due to run-down or run-up) were not studied.

The voltage steps protocol used in cardiomyocytes studies evaluated I_{Ipeak}=Iₚₑₐₖ (V), which was recalculated in CLAMPFIT to G=G(V) as G=I/(V−Eᵣₑᵥ). For HEK cells experiments, the inventors used a ramp protocol (FIG. 17B). The experimental records were I=I(t), where t=time. The inventors transformed these records to I=I(V), which were then further transformed to G=G(V) in CLAMPFIT (FIG. 17B). The parameters of voltage-dependent activation were obtained using a modified Boltzmann distribution. A nonlinear fit with Boltzmann sigmoidal function was done in Prizm (GraphPad).

With regard to single channel patch clamp electrophysiology, cell-attached single-channel recordings were performed at room temperature. Patch pipettes (5-10 MΩ) were pulled from ultra-thick-walled borosilicate glass (BF200-116-10, Sutter Instruments), and coated with Sylgard. Currents were filtered at 2 kHz. The pipette solution contained (in mM): 140 tetraethylammonium methanesulfonate; 10 HEPES; 40 BaCl₂; at 300 mOsm/L, adjusted with tetraethylammonium methanesulfonate; and pH 7.4 adjusted with tetraethylammonium hydroxide. To maintain the membrane potential at 0 mV, the bath contained (in mM): 132 potassium glutamate; 5 KCl; 5 NaCl; 3 MgCl₂; 2 EGTA (ethylene glycol-bis(β-aminoethyl ether)-N,N,N′,N′-tetraacetic acid); 10 glucose; 20 HEPES; at 300 mOsm/L adjusted with glucose; and pH 7.4 adjusted with NaOH. Cell-attached single-channel currents were measured during 200 ms voltage ramps between −80 to +70 mV (portions between −50 and 40 mV displayed and analyzed). For each patch, the inventors recorded 80-120 sweeps with a repetition interval of 10 seconds.

For the flow cytometric FRET assay, HEK293 cells (ATCC) CRL1573 were cultured in 12 well plates and transfected with polyethylenimine (PEI) 25 kDa linear polymer (Polysciences #2396602). Briefly, 1.5 pg of Cerulean (Cer) and Venus (Ven)-tagged protein pairs were mixed together in 100 μl of serum-free DMEM media and 6 μl of PEI is added to each sterile tube. FRET experiments were performed 1-day post-transfection. Protein synthesis inhibitor cyclohexamide (100 μM) was added to cells 2-hrs prior to experimentation to halt synthesis of new fluorophores to allow existing fluorophores to fully mature.

For FRET measurements, the inventors utilized an LSR II (BD Biosciences) flow cytometer equipped with 405 nm, 488 nm, and 633 nm lasers for excitation and 18 different emission channels. Forward and side scatter signals were detected and used to gate for single and healthy cells. For determining FRET efficiency, the inventors measured three distinct fluorescence signals: (1) S_{Cer} corresponding to cerulean emission is measured through the BV421 channel (excitation, 405 nm; emission, 450/50), (2) S_{Ven} corresponding to venus emission measured via FITC channel (excitation, 405 nm; dichroic, 505LP; emission, 525/50), and (3) S_{FRET} corresponding to FRET emission via the BV510 channel (excitation, 405 nm; dichroic, 505LP; emission, 525/50). These raw fluorescence measurements were subsequently used to obtain Ven_{direct} (venus emission due to direct excitation), Cer_{direct} (cerulean emission due to direct excitation), and Ven_{FRET} (venus emission due to FRET excitation). Flow cytometric signals were collected at medium flow rate (2 k-8 k events/sec). Fluorescence data was exported as FCS 3.0 files for further processing and analysis using custom MATLAB functions (MathWorks).

For each experimental run on the flow cytometer, the inventors performed several control experiments: (1) Background fluorescence level for each fluorescent channel (BG_{Cer}, BG_{Ven}, and BG_{FRET}) was obtained by measuring fluorescence from cells exposed to PEI without any fluorophore containing plasmids. (2) Cells expressing Ven fluorophore alone were utilized to measure spectral crosstalk parameter R_{A1}, corresponding to bleed-through of Ven fluorescence into the FRET channel. (3) Cells expressing cerulean fluorophore alone were used to measure spectral crosstalk parameter R_{D1} and R_{D2}, corresponding to bleed-through of cerulean fluorescence into FRET and Ven channels respectively. (4) FRET measurements also require determination of instrument-specific calibration parameters g_{Ven}/g_{Cer} and f_{Ven}/f_{Cer}, corresponding to ratios of fluorescence excitation and emission for Ven to cerulean fluorophores. These parameters also incorporate fluorophore-dependent aspects including, molar extinction (for g) and quantum yield (foil) as well as instrument specific parameters including laser power, attenuation by optical components, as well as photodetection, amplification, digitiziation of fluorescence. For determination of these parameters, the inventors utilized Cer-Ven dimers with four different linker lengths (5, 32, 40, and 228). (5) Coexpression of cerulean and Ven fluorophores provided estimates for concentration-dependent collisional FRET.

For the above experiments, R_{A1}˜0.11, R_{D1}˜2.8, and R_{D2}˜0.006. The inventors observed only minor day-to-day variation in these measurements. For each cell, spectral cross-talk was subtracted by applying the following matrix operation:

$$[ \begin{matrix}{Cer}_{direct} \\ {Ven}_{direct} \\ {Ven}_{FRET}\end{matrix} ] = {[ \begin{matrix}1 / {R}_{D 1} & 0 & 0 \\ {R}_{D 2} / {R}_{D 1} & 1 / {R}_{A 1} & 0 \\ 1 & 1 & 1\end{matrix} ]}^{- 1} [ \begin{matrix}{S}_{Cer} \\ {S}_{Ven} \\ {S}_{FRET}\end{matrix} ] .$$

Following spectral unmixing, the inventors obtained g_{Ven}/g_{Cer} and f_{Ven}/f_{Cer} from data for Cer-Ven dimers by determining the slope and intercept for the following linear relationship:

$$\frac{{Ven}_{FRET}}{{Cer}_{direct}} = \frac{{g}_{Cer}}{{g}_{Ven}} \cdot \frac{{Ven}_{direct}}{{Cer}_{direct}} - \frac{{f}_{Ven}}{{f}_{Cer}} .$$

For typical experiments, g_{Ven}/g_{Cer}=0.0194 and f_{Ven}/f_{Cer}=2.3441. Having obtained these calibration values, donor-centric FRET efficiencies were computed as:

$${E}_{D} = \frac{{Ven}_{FRET}}{{Ven}_{FRET} + \frac{{f}_{Ven}}{{f}_{Cer}} \cdot {Cer}_{direct}} .$$

For Cer-Ven dimers, FRET efficiencies of ˜0.55, 0.38, and 0.05 were obtained for linker lengths 5, 32, and 228 respectively. The relative proportion of Cerulean and Venus fluorophores in each cell was determined as N_{Cer}=Cer_{direct}/(1−E_{D}) and N_{Ven}=Ven_{direct}/(g_{Ven}/g_{Cer}×f_{Ven}/f_{Cer}). To construct FRET 2-hybrid binding curves, the inventors imposed a 1:1 binding isotherm. For each FRET pairs, K_{d,EFF} and E_{D,max} and 95%—confidence intervals were obtained by constrained least squares fit.

For the proximity labeling, isolated ventricular cardiomyocytes were incubated in labeling solution with 0.5 μM biotin-phenol (Iris-biotech) for 30 minutes. During the final 10 minutes of labeling, 1 μM isoproterenol (Sigma 15627) was added. To initiate labeling, H₂O₂ (Sigma H1009) was added to a final concentration of 1 mM for 1 min Exactly 1 minute after H₂O₂ treatment, the cells were washed three times with cold quenching solution containing (in mM) 10 Sodium ascorbate (VWR 95035-692), 5 Trolox (Sigma 238813), and 10 Sodium azide (Sigma S2002). After the cells were harvested by centrifugation, the quenching solution was aspirated and the pellet was flash-frozen and stored at −80° C. until streptavidin pull-down.

For biotinylation in Langendorff-perfused hearts, mice were injected with 5 mg/kg propranolol-HCl (Sigma PHR1308) to suppress adrenergic stimulation during the isoflurane anesthesia and cardiectomy. Hearts were retrograde perfused with Krebs' solution for 10 minutes prior to addition of biotin-phenol for 15 minutes. During the final 5 minutes, 1 μM Isoproterenol (Sigma 1351005) or vehicle was added to the perfusate. The hearts were then perfused with 1 mM H₂O₂ for 1 minute, followed by 3-minutes of perfusion of quenching solution. Electrocardiograms were monitored throughout the experiment to ensure viability of the preparation and an isoproterenol-induced increase in heart rate.

The cells or whole heart tissue were lysed with a hand-held tip homogenizer in a solution containing (in mM), 50 Tris (tris(hydroxymethyl)aminomethane), 150 NaCl, 10 EGTA, 10 EDTA, 1% Triton X-100 (v/v), 0.1% SDS (w/v), 10 Sodium ascorbate, 5 Trolox, and 10 Sodium azide, phosphatase inhibitors (Sigma 4906845001), protease inhibitors (Sigma 4693159001), Calpain inhibitor I (Sigma A6185) and Calpain inhibitor II (Sigma A6060). Biotin labeling of the samples was confirmed by Western blot with Streptavidin-HRP (Sigma RABHRP3) and the response to isoproterenol was assessed by immunoblotting with a phospho-phospholamban (Ser16/Thr17) antibody (Cell Signaling #8496).

With regard to immunoprecipitation and immunoblots, cardiomyocytes were lysed with a hand-held tip homogenizer in a 1% (v/v) Triton X-100 buffer containing (in mM): 50 Tris-HCl (pH7.4) 150 NaCl, 10 EDTA, 10 EGTA and protease inhibitors. The lysates were incubated on ice for 30 minutes, centrifuged at 14 k RPM at 4° C. for 10 minutes and supernatants collected. Proteins were size-separated on SDS-PAGE, transferred to nitrocellulose membranes, and probed with anti-V5 antibody (Fisher R960-25), an anti-α_{1C} antibody, a custom-made polyclonal anti-13 antibody (epitope: mouse residues 120-138: DSYTSRPSDSDVSLEEDRE), an anti-JPH2 antibody (Pierce), an anti-calmodulin antibody (Millipore Sigma 05-173), an anti-RyR2 antibody, and an anti-K_{V}1.5 antibody (Alomone, APC-150). Anti-FLAG antibody (Sigma) immunoprecipitations were performed overnight in a lysis buffer consisting of (in mM): 50 Tris-HCl pH 7.4, 150 NaCl, 0.25% Triton X-100 (v/v), 10 EDTA, 10 EGTA, 0.01 Calpain inhibitor I, 0.01 Calpain inhibitor II, and Complete protease inhibitors (1 per 7 ml, Roche). Antibody-protein complexes were collected using protein A conjugated agarose (Amersham) for 2 h, followed by at least 3 washes in lysis buffer. Proteins were size-separated by SDS, transferred to nitrocellulose membranes and probed with HRP-conjugated anti-FLAG (Sigma) antibody, a custom-made anti-13 antibody and HRP-conjugated secondary goat anti-rabbit antibody. Phospholamban phosphorylation was detected with an anti-phospho-PLB antibody (Cell Signaling #8496). Detection of luminescence was performed with a CCD camera (Carestream Imaging).

For immunofluorescence, isolated cardiomyocytes were first exposed to biotin-phenol and H₂O₂, as described above. After quenching, the cells were fixed for 15 minutes in 4% paraformaldehyde, washed with glycine/PBS (phosphate-buffered saline) twice, treated with PBST (0.1% Triton X-100 (v/v) in PBS) for 5 minutes, and blocked with 3% BSA (w/v) in PBS for 1 hour. Indirect immunofluorescence was performed using a 1:500 anti-V5 antibody (Fisher R960-25) and 1:1000 Alexa594-labeled goat-anti-mouse antibody (Fisher A-11032), and 1:800 streptavidin-Alexa Fluor 488 conjugate (Fisher S32354). Images were acquired using a confocal microscope.

With regard to the processing of biotinylated proteins for mass spectrometry, proteins were precipitated with Trichloroacetic acid (TCA; Sigma T9159) and then centrifuged at 21,130×g at 4° C. for 10 minutes. The pellet was washed with −20° C. cold acetone (Sigma 650501), vortexed, and centrifuged at 21,130×g at 4° C. for 10 minutes. Following centrifugation, acetone was aspirated and the pellet was acetone-washed again three more times. After the last washing step, the pellet was resuspended in: 8M urea, 100 mM sodium phosphate pH 8, 100 mM NH₄HCO₃, and 1% SDS (w/v) and rotated at room temperature until fully dissolved. Re-suspended proteins were centrifuged at 21,130×g at room temperature for 10 minutes and the cleared supernatant was transferred to a new microcentrifuge tube. To reduce disulfides, 10 mM TCEP-HCl (Thermo Fisher Scientific PG82089) in Milli-Q water titrated to pH 7.5 with NaOH was added. To alkylate free Cys, freshly prepared 400 mM iodoacetamide (Thermo Fisher Scientific 90034) stock solution in 50 mM ammonium bicarbonate was added to the supernatant to a final concentration of 20 mM, immediately vortexed, and incubated in the dark for 25 minutes at room temperature. After alkylation, freshly prepared DTT (dithiothreitol) stock solution was added to 50 mM final concentration to quench alkylation. Water was added to each sample to reach a final concentration of 4 M urea and 0.5% (w/v) of SDS.

A 100 μL suspension equivalent per sample of streptavidin magnetic beads (Thermo Fisher Scientific #88817) was washed twice with 4 M urea, 0.5% SDS (w/v), 100 mM sodium phosphate pH 8 and was added to each sample (about 1 mg), diluting each sample with an equal amount of water to reach a final concentration of 2 M urea, 0.25% SDS (w/v), 50 mM sodium phosphate pH 8 during pulldown. The tubes were rotated overnight at 4° C. Following streptavidin pull-down, the magnetic beads were washed three times with 4 M urea, 0.5% SDS (w/v), 100 mM sodium phosphate pH 8, and three times with the same buffer without SDS. The beads were transferred to new tubes for the last wash step. Before final pulldown of the beads for mass spectrometry analysis, 5% of beads resuspension was removed for streptavidin-HRP blotting.

For on-bead digestion and TMT labeling, the liquid reagents used were HPLC quality grade. Washed beads were re-suspended in 50 μL of 200 mM EPPS (4-(2-Hydroxyethyl)-1-piperazinepropanesulfonic acid) buffer pH 8.5, 2% acetonitrile (v/v) with 1 μL of LysC stock solution (2 mg/ml, Wako), vortexed briefly and incubated at 37° C. for 3 hr. Then, 50 μL of trypsin stock (Promega #V5111) diluted 1:100 (v/v) in 200 mM EPPS pH 8.5 was added. After mixing, digests were incubated at 37° C. overnight and beads were magnetically removed. Peptides were directly labeled after digest. For this, acetonitrile was added to a concentration of 30% (v/v) and peptides were labeled with TMT 10-plex reagents (Thermo Fisher Scientific #90406) for 1 hr. Reactions were quenched with hydroxylamine at a final concentration of 0.3% (v/v) for 15 minutes and 1% of labeled peptides were analyzed for efficiency of label incorporation and relative ratios by mass spectrometry. After quenching, peptide solutions were acidified with formic acid, trifluoroacetic acid (TFA) was added to a concentration of 0.1% and peptides were desalted and fractionated by high pH reversed phase chromatography (Thermo Fisher Scientific #84868). After loading of labeled peptides onto pre-conditioned columns and a single wash with water, excess unincorporated TMT label was removed by washing reversed phase columns once with 0.1% trimethylamine (TEA) buffer containing 5% acetonitrile. Samples were fractionated under alkaline conditions into 12 fractions with increasing concentrations of acetonitrile: 10%, 12.5%, 15%, 17.5%, 20%, 25%, 30%, 35%, 40%, 50%, 65% and 80%. Fractions 1 and 7, 2 and 8, 3 and 9, 4 and 10, 5 and 11, 6 and 12 were pooled to obtain 6 final pooled fractions for subsequent analysis. Pooled fractions were dried to completion and further purified and desalted by acidic C₁₈ solid phase extraction (StageTip). Labeled peptides were finally re-suspended in 1% formic acid (v/v) and 3% acetonitrile (v/v).

With regard to whole cell proteomics, specifically cell lysis, protein digest and TMT labeling mass spectrometry analysis, the cells were lysed by homogenization (QIAshredder cartridges, Qiagen) in lysis buffer (2% SDS, 150 mM NaCl, 50 mM Tris pH 7.4). Lysates were reduced with 5 mM DTT, alkylated with 15 mM iodoacetamide for 30 minutes in the dark, alkylation reactions quenched with freshly prepared DTT added to a concentration of 50 mM and proteins precipitated by methanol/chloroform precipitation. Digests were carried out in 1M urea freshly prepared in 200 mM EPPS pH 8.5 in presence of 2% acetonitrile (v/v) with LysC (Wako, 2 mg/ml, used 1:75 w/w protease:substrates during digest) for 3 hours at room temperature and after subsequent addition of trypsin (Promega #V5111, stock 1:100 w/w protease:substrates) over night at 37° C. Missed cleavage rate was assayed from a small aliquot by mass spectrometry. For the whole proteome analysis, digests containing approximately 60 μg of peptide material were directly labeled with TMT reagents (Thermo Fisher Scientific). Labeling efficiency and TMT ratios were assayed by mass spectrometry, while labeling reactions were stored at −80° C. After quenching of TMT labeling reactions with hydroxylamine, TMT labeling reactions were mixed, solvent evaporated to near completion and TMT labeled peptides purified and desalted by acidic reversed phase C₁₈ chromatography. Peptides were then fractionated by alkaline reversed phase chromatography into 96 fractions and combined into 24 samples.

For the mass spectrometry analysis, data collection followed a MultiNotch MS³ TMT method using an Orbitrap Lumos mass spectrometer coupled to a Proxeon EASY-nLC 1200 liquid chromatography (LC) system (both Thermo Fisher Scientific). The capillary column used was packed with C₁₈ resin (35 cm length, 75 μm inner diameter, matrix 2.6 μm Accucore (Thermo Fisher Scientific)). Peptides of each fraction were separated for 4 hours over acidic acetonitrile gradients by LC prior to mass spectrometry (MS) analysis. The scan sequence started with an MS' scan (Orbitrap analysis; resolution 120,000; mass range 400-1400 Th). MS² analysis followed collision-induced dissociation (CID, CE=35) with a maximum ion injection time of 150-300 ms and an isolation window of 0.4 m/z. In order to obtain quantitative information, MS³ precursors were fragmented by high-energy collision-induced dissociation (HCD) and analyzed in the Orbitrap at a resolution of 50,000 at 200 Th.

Peptides were searched with SEQUEST (v.28, rev. 12) based software against a size-sorted forward and reverse database of the M. musculus proteome (Uniprot 07/2014) with added common contaminant proteins. For this, the spectra were first converted to mzXML. Searches were performed using a mass tolerance of 20 ppm for precursors and a fragment ion tolerance of 0.9 Da. For the searches, maximally 2 missed cleavages per peptide were allowed. The inventors searched dynamically for oxidized methionine residues (+15.9949 Da) and, where indicated, for phospho-modification of S,T and Y residues (+79.9663 Da). The inventors applied a target decoy database strategy and a false discovery rate (FDR) of 1% was set for peptide-spectrum matches following filtering by linear discriminant analysis (LDA). The FDR for final collapsed proteins was 1%. MS¹ data were calibrated post search and searches performed again. A modified version of the Ascore algorithm was used to quantify the confidence assignment of phosphorylation sites. Phosphorylation localized to particular residues required Ascore values >13 (p≤0.05) for confident localization. Quantitative information on peptides was derived from MS³ scans. Quant tables were generated requiring an MS² isolation specificity of >70% for each peptide and a sum of TMT signal to noise (s/n) of >200 over all channels for any given peptide and exported to Excel and further processed therein.

The relative summed TMT s/n for proteins between two experimental conditions (referred to as “enrichment” above) was calculated from the sum of TMT s/n for all peptides quantified of a given protein. For gene ontology (GO) term enrichment, the BINGO package in Cytoscape was used. Scaled quantification data were subjected to two-way clustering (JMP software package) and changes in enrichment were analyzed using Graphpad Prism 8 (Graphpad Software). FDR corrected p-values were used for volcano plots.

With regard to the purification of GFP-conjugated Rad and the phosphoproteomic analysis, HEK293T cells were cultured in DMEM with 10% FBS and 1% Pen-Strep. Cells were transfected with GFP-Rad using lipofectamine 2000, as described above. Media was changed 4-6 hours after transfection. After 24 hours, the cells were treated with trypsin and spun down for 5 minutes at 1000 rpm. The cells were then resuspended in PBS with 10 μM forskolin for 5 minutes. After washing the cell pellets three times with phosphate-buffered saline (PBS), the cells were frozen at −80° C. The cold cell pellets were lysed in PBS with 0.1% triton X-100 (v/v, Sigma) and a phosphatase inhibitor mixture (PhosSTOP, Roche) by pipetting up and down several times. The lysates were homogenized by passing them through QIAshredder cartridges (Qiagen) and incubated with GFP-trap agarose beads (Chromotek, Germany) for 4 hours at 4° C. with constant rotation. Beads were washed 3 times with PBS with 0.1% (v/v) Triton and three times with detergent-free PBS and subjected to on-bead digest with trypsin (Promega #V5111), LysC (Wako) or ArgC (Promega #V1881, ArgC digestion buffer 50 mM Tris-HCL pH 7.8, 5 mM CaCl₂, 2 mM EDTA, 2% acetonitrile (v/v)) separately, as described above, overnight at 37° C. After acidification, peptides were purified by reversed phase C₁₈ chromatography and subjected to MS/MS analysis. For this, the same parameters as given above for the MS' and MS² scans were used with an isolation window of 1.2 Da and taking neutral loss of 97.9763 Da into account with multi-stage activation (MSA) set for MS² scans. Analysis of phospho-site localization was performed as described above.

For the statistical analysis, results are presented as mean±SEM. For comparisons between two groups, the Student's t-test was used. Statistical analyses were performed using Prism 8 (Graphpad Software). For multiple group comparisons, a one-way ANOVA followed by either Dunnett's or Tukey's post-hoc test were performed using Prism 8. Differences were considered statistically significant at values of P<0.05.

Ca²⁺ influx through L-type Ca_{V}1.2 channels into cardiomyocytes initiates excitation-contraction coupling by triggering Ca²⁺ release from ryanodine receptors, and modulates action potential duration and gene expression. In the failing heart, activation of Ca_{V}1.2 channels can trigger electrical instability, early after-depolarizations, arrhythmias, and sudden death. Moreover, increased activation of Ca_{V}1.2 channels can elicit Ca²⁺-responsive signaling pathways, including those affecting gene expression, which contributes to the pathogenesis of heart failure and hypertrophy.

Voltage-gated Ca²⁺ channels are multi-subunit protein complexes comprised of a pseudo-tetrameric pore-forming α₁ subunit and a cytosolic β subunit that interacts with the α₁ subunit at its α-interaction domain (AID) within the intracellular linker between domains I and II. This interaction and its supporting molecular interfaces has emerged as a central hub that organizes a sophisticated scheme of ion channel regulation that is critical for the maintenance of proper cardiac function. In heterologous cells, binding to the β subunit is obligatory for α_{1C} trafficking to the plasma membrane, and for normalizing channel activation and inactivation gating properties via a mechanism that requires a rigid IS6-AID helix linker.

Following activation, the channel undergoes Ca²⁺-dependent inactivation, which is dependent upon calmodulin (CaM), and voltage-dependent inactivation, which is dependent upon the β subunit, the I-II linker and the cytosolic ends of the S6 transmembrane segments. Cardiac Ca_{V}1.2 channels are prominently up-regulated by (3-adrenergic agonists via activation of protein kinase A (PKA). This regulation, a component of the physiological ‘fight-or-flight’ response, contributes to the increased contractility of the heart during exercise. The overall features of this regulation—PKA-dependent enhanced whole-cell current amplitude, leftward shift in voltage-dependence of channel activation, and increased single-channel channel open probability (Pₒ)—are well-established, yet essential molecular details remained stubbornly enigmatic. Recently, we showed that Ca_{V}β subunits are required for β-adrenergic regulation of Ca_{V}1.2 channels and positive inotropy in the heart. Further, by using proximity proteomics and multiplexed quantitative mass spectrometry, and cellular electrophysiology, we found that the Ca²⁺ channel inhibitor Rad²⁸, which binds to the β subunit, is the functionally relevant PKA target, imparting β-adrenergic regulation upon Ca_{V}1.2.

Beyond these, the I-II loop is also subject to alternative splicing that tunes channel function and interacting proteins in a cell-type specific manner. The inclusion of alternatively-spliced exon 9* is observed at high levels in the smooth muscle and at lower but variable expression in adult heart that increases in animal models of hypertrophy and in the peri-infarct zone after MI in mice. This variant results in the insertion of a 25 amino acid residues C-terminal to the AID and tunes channel activation. In all, the modulatory landscape supported by the Ca_{V}1.2 domain I-II linker appears rich and multifaceted, involving the β subunits, RGK proteins, phosphoregulation by PKA, and alternative splicing, all poised to precisely tune Ca²⁺ influx into cardiomyocytes.

Several important mechanistic unknowns persist impeding in-depth pathophysiological understanding. First, although Ca_{V}β-α₁ interaction is obligatory for Ca_{V}1.2 trafficking in heterologous cells, we found it to be dispensable for cell surface trafficking, for basal function and excitation-contraction coupling in adult cardiomyocyte, thus raising fundamental questions about the functional role of Ca_{V}β subunits in cardiomyocytes. Second, it is unknown how distal conformational changes involving Rad interaction with the Ca_{V}β subunit and phosphorylation-dependent signaling are ultimately conveyed to the channel pore-domain. Third, how alternative splicing of the domain I-II linker contributes to this overall regulatory scheme including effects of phosphorylation remains to be fully-elucidated. Importantly, the pathogenesis of heart failure has been long-suspected to reshape this regulatory framework, although the precise changes remain largely undefined.

To dissect these possibilities, we measured baseline channel gating properties and the strength of adrenergic modulation of Ca_{V}1.2 in three transgenic mouse models: (1) Our previously-established AID mutant where Ca_{V}1.2 is incapable of binding to the β subunit, (2) Triple-glycine substitution (GGG-α_{1C}) of the rigid I-II linker, which connects the pore-domain with the AID. These mutations have been previously shown to disrupt coupling between these two domains, and (3) The insertion of 25 amino acid residues C-terminal to the AID corresponding to the introduction of exon 9* variant (9*-α_{1C}). We found that the loss of β subunit binding diminishes channel Pₒ by destabilizing high-activity gating mode. In like manner, increased flexibility of the IS6-AID rigid linker imparted by triple-glycine substitution also reduced basal Pₒ by preventing sojourns to the high activity gating mode and abolished stimulation by β-adrenergic agonists. By contrast, we found that insertion of the 9* exon conferred the opposite properties, namely an increase in basal channel Pₒ by increasing channel availability and stabilizing the high-activity gating mode, and a preservation of physiological responses to the β-adrenergic signaling cascade. Interestingly, this functional signature is reminiscent of Ca_{V}1.2 behavior in myocytes from failing human hearts, which also show increased availability and Pₒ. Indeed, we found that myocytes of patients with end-stage heart failure (HF) show increased splice inclusion of exon 9* suggesting that molecular alterations in I-II loop may be pathophysiologically important. Taken together, these findings identify the IS6-AID linker as a vital molecular element for transducing the PKA-induced activation of Ca_{V}1.2, and as a molecular rheostat for Ca_{V}1.2 activity whereby distinct structural changes elicited by β subunits, RGK proteins, and alternative splicing bidirectionally tune Ca²⁺ influx into the heart in both normal physiology and during heart failure.

To study cardiac Ca_{V}1.2 channels in their native context, we generated transgenic mice featuring inducible, cardiac-specific expression of dihydropyridine (DHP)-resistant, FLAG-epitope-tagged α_{1C}. We have previously shown that control transgenic FLAG-tagged DHP-resistant α_{1C} subunits, termed pseudo-wild-type (pWT) α_{1C} have similar properties as native cardiac Ca²⁺ channels. To study properties of Ca_{V}1.2 devoid of the 13 subunit, we used transgenic mice expressing rabbit α_{1C} with a disrupted AID via alanine substitutions of 3 conserved residues, Y467, W470 and 1471, that are essential for binding of β subunit. We further generated transgenic mice expressing FLAG-tagged DHP-resistant α_{1C} with a substitution of three glycine residues (GGG) for the AKA motif in the IS6-AID linker, designated GGG-α_{1C}. All three lines, pWT-, AID- and GGG-a_{1C} were crossed with α-myosin heavy chain reverse transcriptional transactivator (αMHC-rtTA) transgenic mice. Several GGG-α_{1C} and AID-mutant founder transgenic lines were generated, and all lines demonstrated doxycycline-induced α_{1C} expression after crossing with the αMHC-rtTA mice. As expected, channels with a disrupted AID motif do not bind β subunits, assessed by anti-FLAG antibody immunoprecipitation of cleared homogenates. By comparison, GGG-α_{1C} channels exhibit robust β subunit binding similar to pWT α_{1C}.

Previously, we showed that β-less Ca_{V}1.2 channels traffic to the dyad and produce currents that mediate normal E-C coupling in cardiomyocytes. To determine whether β binding to α_{1C} alters basal channel gating in cardiomyocytes, we utilized low-noise single-channel recordings of acutely isolated cardiomyocytes. Stochastic channel openings, which reflect near-steady-state open probability (Pₒ) at each voltage, were elicited by a slow voltage ramp. Ca²⁺ channels in cardiomyocytes from non-transgenic mice were inhibited by 300 nM nisoldipine. In the transgenic mice, however, there is a mixture of transgenic nisodipine-resistant channels and endogenous nisoldipine-sensitive channels. As dihydropyridines are known to allosterically modify channel gating, partial blockade of nisoldipine-resistant channels may be a confounding factor. To obviate this possibility, we obtain ˜80-120 stochastic records from each patch and subsequently apply nisoldipine to identify resistant transgenic channels. This process allows us to unambiguously establish baseline function of mutant Ca_{V}1.2. Indeed, exemplar traces from DHP-resistant pWT channels confirm channel openings in the presence of nisoldipine. Measurements of steady-state Pₒ as a function of voltage were obtained by averaging many records and by normalizing the unitary current level. Reassuringly, steady-state Pₒ-V relationships of pWT channels are similar to that of non-transgenic channels. The DHP-resistant AID-mutant α_{1C} channels, however, displayed a striking 3.5-fold reduction in maximal Pₒ compared to DHP-sensitive endogenous Ca²⁺ channels from non-transgenic Ca²⁺ mice and DHP-resistant channels from pWT mice. To further elucidate changes in elementary channel gating mechanisms, we scrutinized single-trial average open probabilities (P̅ₒ) from one-channel patches. A dash-line discriminator with P̅ₒ=0.1 was used to identify low activity versus high-activity traces. Thus analyzed, pWT channels switched between epochs of no openings or blanks (40.6% of traces), low activity (33.0%), and high activity (26.4%) consistent with previous studies. By comparison, AID-mutant α_{1C} channels exhibited a distinct pattern (p<0.0001 by χ² test of independence) with rare sojourns to the high-activity mode (7.1%) and a higher propensity for blank (45.7%) and low activity sweeps (47.2%). Thus β subunit binding appears to stabilize the high-activity gating mode. We conclude that the binding of the β subunit to the α_{1C} subunit critically modulates Ca_{V}1.2 channel activity in cardiac myocytes by enhancing channel openings.

Having established the importance of β subunits in upregulating Ca_{V}1.2 channel activity, we considered whether the rigid I-II linker between the AID and the IS6 pore helix is essential for tuning channel function. Introduction of the three glycine residues in the IS6-AID linker had no effect on α_{1C} subcellular localization and functional expression in cardiomyocytes Immunofluorescence studies using anti-FLAG antibody on fixed cardiomyocytes showed that GGG-α_{1C} Ca_{V}1.2 channels demonstrated a striated z-disk pattern consistent with localization to the surface membrane and transverse-tubules (t-tubules). Similar to cardiomyocytes expressing pWT or AID-mutant α_{1C} transgenic channels, field-stimulated contraction of cardiomyocytes isolated from GGG-α_{1C} transgenic mice persisted in the presence of 300 nM nisoldipine, which is sufficient to block excitation-contraction coupling induced by endogenous Ca_{V}1.2 channels in non-transgenic mice, indicating that the GGG-α_{1C} Ca_{V}1.2 channels are localized correctly and flux sufficient Ca²⁺ to evoke Ca²⁺-induced Ca²⁺ release in cardiomyocytes.

When GGG-α_{1C} is co-expressed in Xenopus oocytes with β_{2B}, the predominant β₂ isoform in the heart, channels displayed accelerated voltage-dependent inactivation but slowed Ca²⁺-dependent inactivation. Here, we assessed aggregate inactivation of Ca²⁺ channels in the heart with Ca²⁺ as a charge carrier. The inactivation kinetics of nisoldipine-resistant GGG-α_{1C} Ca²⁺ currents were significantly faster at +30 mV test potential compared to pWT controls. Therefore, in adult cardiomyocytes Ca_{V}1.2 channels comprised of transgenic GGG-α_{1C} have faster overall inactivation kinetics as compared to transgenic pWT Ca_{V}1.2 channels likely reflecting accelerated kinetics of voltage-dependent inactivation.

Given that β subunits upregulate Ca_{V}1.2 channel Pₒ, we considered whether disruption of the rigid IS6-AID linker might reverse this effect. Consistent with this possibility, the conductance-voltage (G-V) relationships, normalized to cell capacitance, of nisoldipine-resistant transgenic mutant GGG-α_{1C} channels was reduced compared to pWT. To directly assess changes in Pₒ, we used low-noise single-channel recordings of acutely isolated cardiomyocytes from the GGG-α_{1C} transgenic mice. Exemplar records show DHP-resistant GGG-α_{1C} channels exhibit sparse channel openings, a distinct gating pattern compared to pWT and non-transgenic channels which undergo high-activity flickery openings. Ensemble average Pₒ-V relationship and bar-graph summary of maximal Pₒ from individual patches show a striking 3.5-fold reduction in maximal Pₒ compared to both pWT and non-transgenic channels. Interestingly, this reduced basal activity of GGG-α_{1C} is reminiscent of β-less AID-mutant channels suggesting that disruption of the rigid IS6-AID linker may be akin to uncoupling β subunit from the channel pore. To further scrutinize this possibility, we assessed average Pₒ from individual trials for one channel patches of GGG-α_{1C}. Unlike pWT channels, the single-trial P̅ₒ distribution of the GGG-α_{1C} channels was restricted to either blank (73.4%) or low activity sweeps (26.6%) with no evidence of high activity traces. As GGG-α_{1C} are fully capable of β subunit binding, these results suggest that the rigidity of the linker between the pore-domain and I-II loop may be a structural requirement for the high-activity gating configuration. As AID-mutant channels exhibit some propensity for high-activity traces, one attractive possibility is that the IS6-AID linker may switch between rigid and flexible conformations, with β subunit binding to the AID serving to stabilize the rigid helical linker conformation, an outcome also supported by X-ray crystallographic and circular dichoism experiments.

Having established the loop as a vital regulator of channel openings, we considered whether alternative splicing in this domain might tune channel gating. The 9* exon, which encodes a 75-nucleotide sequence within the is expressed at a high level in aortic smooth muscle and has lower expression in non-diseased adult human and rat heart. The 9* exon-containing channels are however increased in rodent models of hypertrophy and in the perinfarct zone. This altered pattern of α_{1C} splicing in rodent models raises the possibility of pathological inclusion of exon 9* in human cardiac disease, an outcome yet to be observed clinically. As such, we sought to determine whether the frequency of exon 9* splice variant is changed in humans with end-stage heart failure. Samples from patients undergoing LVAD implantation Columbia-NY Presbyterian Hospital were acquired in the operating room, and compared to samples obtained from donor hearts without heart failure. Exon 9* transcript expression was increased in humans with heart failure compared to control samples.

To determine the functional consequence of exon 9* splice inclusion in cardiomyocytes, we created transgenic mice with cardiac-specific expression of DHP-resistant Ca²⁺ channels containing exon 9*, but with all other mutually exclusive exons typical of cardiac variants. The 9*-α_{1C} channels still bound β subunits similar to pWT channels, and trafficked to the surface membrane and t-tubules, as demonstrated by the striated z-disk pattern of immunofluorescence. Whole-cell electrophysiological analysis of nisoldipine-resistant transgenic mutant 9*-α_{1C} channels revealed a significant increase in the G-V relationship normalized to cell capacitance in comparison to pWT channels. Changes in whole cell current may stem from alterations in channel trafficking, unitary conductance, or baseline open probability. To dissect these mechanistic possibilities, we undertook low-noise single-channel recordings to determine whether the increased normalized conductance of 9*-α_{1C} reflected a genuine increase in channel Pₒ. In the presence of nisoldipine, the DHP-resistant 9*-α_{1C} channels exhibited robust channel openings with the ensemble average demonstrating a marked increase in maximal Pₒ (0.26±0.03, mean±s.e.m) in comparison to pWT (0.15±0.015, mean±s.e.m). Furthermore, examination of single-trial Pₒ distribution revealed a virtual elimination of blank traces (˜0% for 9* versus 40.5% for pWT), and increased propensity for the high activity gating mode (56.7%). Thus, the insertion of the 9* exon into the loop upregulates basal voltage-dependent opening of Ca²⁺ channels by enhancing channel availability and stabilizing the high Pₒ gating configuration.

Recently, we determined that the mechanism of adrenergic stimulation of Ca_{V}1.2 requires constitutive pre-inhibition of Ca_{V}1.2 owing to Rad interaction with the Ca_{V} channel β subunit. PKA phosphorylation of Rad at conserved sites in its C-terminus alters its interaction with the Ca_{V} channel 13 subunit and relieves constitutive inhibition. As increased flexibility of the IS6-AID linker effective decouples β subunit mediated regulation, we hypothesized that the rigid IS6-AID linker may be also essential for adrenergic upregulation of Ca_{V}1.2 currents. Consistent with this possibility, at the single channel level, Rad inhibited Ca_{V}1.2 channels have decreased availability and increased propensity for low activity gating mode, akin to GGG-α_{1C} channels.

In cardiomyocytes isolated from mice expressing transgenic pWT α_{1C}, 10 μM forskolin (which stimulates adenylyl cyclase to produce cyclic AMP, thereby activating PKA) increased the nisoldipine-insensitive maximal conductance (Gₘₐₓ) by a mean of 1.4-fold, and shifted the V₅₀ for activation. Ca²⁺ currents through transgenic GGG-α_{1C} channels, in contrast, were not stimulated by forskolin. By comparison, forskolin increased the Gₘₐₓ of 9* exon-containing Ca_{V}1.2 channels by a mean of 1.4-fold, and shifted the V₅₀ for activation suggesting that 9* exon still preserves responsiveness for PKA modulation. For both pWT and 9* Ca_{V}1.2 channels, the forskolin-induced enhancement of Ca²⁺ current was greatest at hyperpolarized potentials and fell as the test potential approached the reversal potential, consistent with prior observations. The GGG Ca_{V}1.2 channels failed to respond to forskolin at any test potential.

We used reconstitution studies in HEK cells to further gain mechanistic insights into the mechanisms by which the β subunit and I-II loop modulate adrenergic regulation of Cav1.2. We co-expressed Rad with α_{1C} and β_{2B} subunits in HEK293T cells, limiting Rad expression by using 1:3 to 1:6 cDNA ratio of Rad:Ca_{V}1.2 subunits in order to avoid eliminating the Ca²⁺ current. Perforated, whole-cell patch clamp was used to preserve the normal intracellular milieu and signaling cascades, and minimize current run-down. In HEK293T cells transfected with only α_{1C}+β_{2B}, superfusion of forskolin over 1-3 minutes had no impact on Ba²⁺ current. In contrast, applying forskolin to cells expressing α_{1C}+β_{2B}+Rad increased the maximal conductance (Gₘₐₓ) by a mean of 1.6-fold, and shifted the V₅₀ for activation. Consistent with our findings in cardiomyocytes, in cells transfected with GGG-α_{1C} forskolin failed to increase Gₘₐₓ or shift the voltage-dependence of activation in a hyperpolarizing direction. In contrast, applying forskolin to cells expressing 9*-α_{1C}, β_{2B}, and Rad increased the Gₘₐₓ by a mean of 1.6-fold, and shifted the V₅₀ for activation. Thus, PKA-modulation of Ca_{V} channels in heart and cardiomyocytes is dependent on both Rad phosphorylation and a rigid IS6-AID linker.

This work has examined mechanisms of regulation of cardiac Ca_{V}1.2 channel gating by three essential factors with important physiological and pathophysiological consequences that converge at the α_{1C} subunit I-II loop—auxiliary Ca_{V}β subunits, sympathetic activation, and alternative splicing. We discuss our findings on these three inter-related regulatory mechanisms in the context of previously published reports.

Many reconstitution studies in heterologous mammalian cells established the idea that auxiliary Ca_{V}β subunits were necessary for trafficking of Ca_{V}1.2 channels to the plasma membrane, and that this depended on high-affinity Ca_{V}β binding to a discrete α₁-interaction domain (AID) in the α_{1C} I-II loop. Beyond trafficking, Ca_{V}β binding also boosted Ca_{V}1.2 Pₒ and produced a hyperpolarizing shift in the voltage-dependence of channel activation. These latter gating effects were deduced to require formation of a rigid helix spanning IS6 and AID because they were selectively eliminated by a triple glycine substitution that disrupts the continuous helix. In adult cardiomyocytes, cardiac-specific excision of the dominant cardiac Ca_{V}β₂ isoform that reduced protein levels by 96% resulted in only a 26% reduction in whole-cell Ca_{V}1.2 current, providing a first hint that, by contrast to heterologous cells, Ca_{V}β binding to α_{1C} may not be obligatory for forming functional Ca_{V}1.2 channels at the cell surface. We explicitly confirmed this by showing that transgenic mice expressing a DHP-resistant α_{1C} mutant that does not bind Ca_{V}β, nevertheless, yielded robust nisoldipine-resistant whole-cell Ca²⁺ currents indicating that the channels made it to the surface sarcolemma. While Ca_{V}β does not appear necessary for surface trafficking of Ca_{V}1.2 in adult cardiomyocytes, it remained unclear whether this also extended to the impact on channel Pₒ. Here, we unambiguously show using single-channel recordings that Ca_{V}β binding to α_{1C} in cardiomyocytes enhances Ca_{V}1.2 channel Pₒ by 3.5-fold. The single-channel gating signature of AID-mutant channels was dominated by null (46%) and low-activity (47%) sweeps, with rare sojourns into a high-activity (7%) gating mode. By contrast, Ca_{V}β-bound WT channels displayed more high-activity sweeps (26%) and correspondingly lower null (41%) and low-activity (33%) gating modes. GGG-α_{1C} channels displayed only blanks and low-activity gating. These results are consistent with the interpretation that in adult cardiomyocytes Ca_{V}β induces high-Pₒ gating by stabilizing a continuous helix linking IS6 to AID. Unbinding of Ca_{V}β (as occurs with AID-mutant) reduces the propensity for helix formation and accordingly decreases fractional occupancy of the high-Pₒ gating mode. GGG-α_{1C} completely dispels the continuous helix and, therefore, these channels do not sojourn into the high-Pₒ mode. In the cryo-electron microscopy structure of the homologous Ca_{V}1.1 channel, comparison of the two conformations, class Ia and II, revealed significant shifts of the C-terminal end of IS6 and the I-II helix of α₁ subunit, and the β subunit. Substitution of the three glycine residues in either one of the two conformations likely alters the conformation and increases the flexibility of the I-II loop and the position of the β subunit.

We recently reported that β-adrenergic regulation of cardiac Ca_{V}1.2 channel requires Ca_{V}β binding binding to α_{1C} I-II loop, and PKA phosphorylation of Rad, a small G-protein that inhibits Ca²⁺ channels via binding to Ca_{V}β subunit. Here, we report that GGG-α_{1C} channels expressed in cardiomyocytes, or reconstituted with Rad in heterologous cells, do not display PKA-mediated up-regulation of whole-cell current density, even though they bind Ca_{V}β. This result suggests the rigid IS6-AID helical linker as another essential requirement for transduction of sympathetic regulation of Ca_{V}1.2 that is essential for the fight or flight response. The exclusively low-Pₒ gating mode of GGG-α_{1C} channels is reminiscent of the behavior of Rad-inhibited channels. It is intriguing to speculate that Rad interaction with Ca_{V}β may structurally alter the IS6-AID linker as a mechanism for channel inhibition.

Finally, we examined the impact of the α_{1C} 9*-splice variant that is elevated in the failing heart. Interestingly, the 9*-α_{1C} channels had increased basal Pₒ and were exclusively recorded in high activity gating modes, with no sojourns into mode 0 gating. Given the proximity of the 9* exon to the AID, and the role of the continuous IS6-AID helix in promoting high activity Cav1.2 gating, one explanation is that the 9* exon may increase basal Pₒ by further stabilizing the rigid IS6-AID helical linker. Another explanation is that insertion of the 9* exon may affect the function of the voltage-sensor domain of domain II. Previous single-channel experiments have indicated that Ca_{V}1.2 channels in failing hearts have an elevated basal Pₒ which was putatively attributed to enhanced phosphorylation of the channel Our results suggest that the increased Ca_{V}1.2 Pₒ observed in heart failure may be due, in part, to the emergence of the alternatively spliced 9*-α_{1C} variant in this condition.

These results provide key insight into the mechanism underlying the β-adrenergic stimulation of Ca²⁺ current and contractility in the heart. Upon β-adrenergic stimulation, PKA phosphorylation of Rad releases the Rad-induced inhibition of the Ca²⁺ channels. The Rad-inhibited channels are the heart's functional reserve of Ca²⁺ channels, likely having minimal effects on excitation-contraction coupling at rest, but primed to respond to β-adrenergic agonists upon release of Rad-induced inhibition. Thus, therapeutic release of Rad-mediated inhibition of Ca²⁺ channels could be inotropic. β subunit binding to the α_{1C} subunit enables transitions to a high Pₒ state, as Ca²⁺ channels with the AID-mutation fail to transition to high Pₒ states and also fail to respond to β-adrenergic stimulation.

It is to be understood that the method of screening for drugs for prevention of pathologic calcium overload in cardiomyocytes is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method of screening for drugs that increase α_{1C} calcium current in cardiomyocytes and increase cardiac contractility, comprising the step of screening a molecular library for one or more candidate molecules which block interaction between Rad and a cardiomyocyte membrane.

2. A method of screening for drugs that increase α_{1C} calcium current in cardiomyocytes and increase cardiac contractility, comprising the step of screening a molecular library for one or more candidate molecules which block interaction between Rad and a Ca_{V}1.2/Ca_{V}β2 complex.

3. A method of screening for drugs that increase α_{1C} calcium current in cardiomyocytes and increase cardiac contractility, comprising the steps of:
generating a stable cell line that expresses α_{1C} and β₂;
transiently transfecting the stable cell line with Rad and a light sensitive ion channel;
adding a calcium sensitive fluorescent dye to the stable cell line;
exposing the stable cell line to light to depolarize each of the cells and activate the α_{1C} calcium channels;
assessing an amplitude of calcium current by measuring an intensity of calcium dye fluorescence.

4. The method of screening for drugs that increase α_{1C} calcium current in cardiomyocytes and increase cardiac contractility as recited in claim 3, wherein the method of screening for drugs that increase α_{1C} calcium current in cardiomyocytes and increase cardiac contractility is performed in the absence of a screening compound.

5. The method of screening for drugs that increase α_{1C} calcium current in cardiomyocytes and increase cardiac contractility as recited in claim 3, wherein the method of screening for drugs that increase α_{1C} calcium current in cardiomyocytes and increase cardiac contractility is performed in the presence of a screening compound.

6. The method of screening for drugs that increase α_{1C} calcium current in cardiomyocytes and increase cardiac contractility as recited in claim 5, further comprising the step of determining that the screening compound increases α_{1C} calcium current in cardiomyocytes and increases cardiac contractility if the intensity of calcium dye fluorescence is above a threshold value.

7. A method of screening for drugs that block interaction between an RGK GTPase protein and a β-subunit of a calcium channel, comprising the steps of:
attaching a first fluorophore to an RGK GTPase protein;
attaching a second fluorophore to a β-subunit of a calcium channel;
exciting one of the first and second fluorophores; and
measuring fluorescence resonance energy transfer (FRET) efficiency to determine interaction between the RGK GTPase protein and the β-subunit of a calcium channel.

8. The method of screening for drugs that block interaction between an RGK GTPase protein and a β-subunit of a calcium channel as recited in claim 7, wherein the RGK GTPase protein is selected from the group consisting of Rad, Rem, Rem2, and Kir/Gem.

9. The method of screening for drugs that block interaction between an RGK GTPase protein and a β-subunit of a calcium channel as recited in claim 8, wherein the β-subunit is selected from the group consisting of CACNB1, CACNB2, CACNB3, and CACNB4.

10. The method of screening for drugs that block interaction between an RGK GTPase protein and a β-subunit of a calcium channel as recited in claim 9, wherein the calcium channel is selected from the group consisting of CACNA1S, CACNA1C, CACNA1D, CACNA1F, CACNA1A, CACNA1B, CACNA1E, CACNA1 G, CACNA1H, and CACNA1I.

11. A method of screening for drugs that increase α_{1C} calcium current in cardiomyocytes and increase cardiac contractility, comprising the step of screening a molecular library for one or more candidate molecules which block interaction between Rad and a Ca_{V}1.2/Ca_{V}β2 complex and between Rad and Ca_{V}β2.

12. A method of screening for drugs that increase α_{1C} calcium current in cardiomyocytes and increase cardiac contractility, comprising the step of screening a molecular library for one or more candidate molecules which block interaction between Rad and Ca_{V}β2.

13. A method of screening for drugs that enhance interaction between an RGK GTPase protein and a β-subunit of a calcium channel to decrease calcium current, reduce calcium overload and reduce arrhythmias, comprising the steps of:
attaching a first fluorophore to an RGK GTPase protein;
attaching a second fluorophore to a β-subunit of a calcium channel;
expressing the RGK GTPase protein and the β-subunit of the calcium channel in a cell line;
expressing a catalytic subunit of PKA in the cell line;
exciting one of the first and second fluorophores; and
measuring fluorescence resonance energy transfer (FRET) efficiency to determine interaction between the RGK GTPase protein and the β-subunit of the calcium channel.

14. A method of screening for drugs that block interaction between an RGK GTPase protein and a β-subunit of a calcium channel, comprising the steps of:
attaching a first fluorophore to an RGK GTPase protein;
attaching a second fluorophore to an integral membrane bound protein;
exciting one of the first and second fluorophores; and
measuring fluorescence resonance energy transfer (FRET) efficiency to determine interaction between the RGK GTPase protein and the integral membrane bound protein.
